(19) United States
(12) Patent Application Publication
(10) Pub. No.: US 2009/0275077 A1
(43) Pub. Date: Nov. 5, 2009

(54) Methods of Obtaining Optically Active Epoxides and Vicinal Diols from Styrene Oxides

(75) Inventors: Adriana Leonora Botes, Stockport (GB); Jeanette Lotter, Edenvale Ridge (ZA); Michel Labuschagne, Reddersburg (ZA); Robin Kumar Mitra, Stockport (GB)

Correspondence Address: FISH & RICHARDSON P.C., P.O. BOX 1022, MINNEAPOLIS, MN, 55440-1022, US

(73) Assignee: Oxyrane (UK) Limited, Machester (GB)

(21) Appl. No.: 12/372,525

(22) Filed: Feb. 17, 2009

Related U.S. Application Data
(63) Continuation of application No. 11/578,820, filed on Aug. 1, 2007, now abandoned.

(30) Foreign Application Priority Data
Apr. 19, 2004 (ZA) 20042960

(51) Int. Cl. C12P 21/00; C12P 17/02; C12P 7/22; C12N 9/14; C12N 1/19; C07H 21/04; C07K 16/14

(52) U.S. Cl. 435/68.1; 435/123; 435/156; 435/195; 435/254.2; 536/23.2; 530/387.9

(57) ABSTRACT

The invention provides yeast strains, and polypeptides encoded by genes of such yeast strains, that have enantiospecific meso-epoxide hydrolase activity. The invention also features nucleic acid molecules encoding such polypeptides, vectors containing such nucleic acid molecules, and cells containing such vectors. Also embraced by the invention are methods for obtaining containing such nucleic acid molecules, and cells containing such vectors. Also embraced by the invention are methods for obtaining

[symbol]

### TECHNICAL FIELD

This invention relates to biocatalytic reactions, and more particularly to the use of enantiomer selective hydrolases to obtain optically active epoxides and vicinal diols.

### BACKGROUND

Optically active epoxides and vicinal diols are versatile fine chemical intermediates for use in the production of pharmaceuticals, agrochemicals, ferro-electric liquid crystals and flavours and fragrances. Epoxides are highly reactive electrophiles because of the strain inherent in the three-membered ring and the electronegativity of the oxygen. Epoxides react readily with various O-, N-, S-, and C-nucleophiles, acids, bases, reducing and oxidizing agents, allowing the production to bifunctional molecules. Vicinal diols, employed as their highly reactive cyclic sulfites and sulfates, act like epoxide-like synthons with a broad range of nucleophiles. The possibility of double nucleophilic displacement reactions with amidines and azide, allow access to dihydroimidazole derivatives, aziridines, diamines and diazides. Since enantiopure epoxides and vicinal diols can stereospecifically be interconverted, they can be regarded as synthetic equivalents.

Styrene oxide derivatives are among the most useful terminal epoxides from a synthetic standpoint. For example, Styrene oxide and phenylethanediol derivatives with substitutents in the meta- and/or para-position of the phenyl ring can readily be aminated to corresponding substituted phenylethanolamines, and many of these serve as drugs that are currently drawing interest, especially in their optically pure (R)-configurations. In particular, optically active 3-chlorostyrene oxide and 2-(3-chlorophenyl)-1,2-ethanediol can serve as useful synthons for elaboration into pharmaceuticals (Harada et al., 2003, Manoj et al., 2001; Monterde et al., 2004).

Epoxide hydrolases (EC 3.3.2.3) are hydrolytic enzymes that convert epoxides to vicinal diols by ring-opening of the epoxide with water. Epoxide hydrolases are present in mammals, plants, insects and microorganisms.

### SUMMARY

The invention is based in part on the surprising discovery by the inventors that certain microorganisms express epoxide hydrolases with high enantioselectivity. These microorganisms and the yeast enantioselective styrene epoxide hydrolase (YESH) polypeptides of the invention selectively hydrolyse specific enantiomers of styrene oxide (also known as styrene-type epoxide). The genomes of the microorganisms encode polypeptides having highly enantioselective styrene oxide hydrolase activity. Styrene oxides (styrene-type epoxides) are for convenience generally referred to herein as “styrene epoxides” (“SEO”).

As used herein, the terms “styrene epoxide” (SEQ) and “styrene vicinal diol” (or “phenylethanediol” (PED)) generally refer to an unsubstituted or a substituted (mono-, oligo-, or multi-substituted) “styrene-type epoxide” and an unsubstituted or a substituted “styrene-type vicinal diol” (or an unsubstituted or a substituted “phenylethanediol”), respectively. When an epoxide or vicinal diol (or phenylethanediol) is or is not is substituted, it is so stated.

More specifically, the invention provides a process for obtaining an optically active epoxide and/or an optically active vicinal diol, which process includes the steps of: providing an enantiomeric mixture of a styrene epoxide (SEQ); creating a reaction mixture by adding to the enantiomeric mixture a polypeptide, or a functional fragment thereof; having enantioselective styrene epoxide hydrolase activity, the polypeptide being a polypeptide encoded by a gene of a yeast cell or a gene derived from a yeast cell; incubating the reaction mixture; and recovering from the reaction mixture: (a) an enantiopure, or a substantially enantiopure, phenylethanediol (PED); (b) an enantiopure, or a substantially enantiopure, styrene epoxide; or (c) an enantiopure, or a substantially enantiopure, phenylethanediol and an enantiopure, or a substantially enantiopure, styrene epoxide.

Another aspect of the invention is a process for obtaining an optically active epoxide and/or an optically active vicinal diol, which process includes the steps of providing an enantiomeric mixture of a styrene epoxide; creating a reaction mixture by adding to the enantiomeric mixture a cell comprising a nucleic acid encoding, and capable of expressing, a polypeptide having enantioselective styrene epoxide hydrolase activity, the polypeptide being a polypeptide encoded by a gene of a yeast cell; incubating the reaction mixture; and recovering from the reaction mixture: (a) an enantiopure, or a substantially enantiopure, phenylethanediol; (b) an enantiopure, or a substantially enantiopure, styrene epoxide; or (c) an enantiopure, or a substantially enantiopure, phenylethanediol and an enantiopure, or a substantially enantiopure, styrene epoxide.

In both of processes, the incubation can result in the selective production of a phenylethanediol (PED) having the chirality of the enantiomer for which the epoxide hydrolase has selective activity and/or the selective enrichment, relative to the total amount of both enantiomers of the SEO in the mixture, of the SEO enantiomer for which the epoxide does not have selective activity.

The following embodiments apply to both of the above processes. The cell can be a yeast cell. The polypeptide can be encoded by an endogenous gene of the cell or the cell can be a recombinant cell, the polypeptide being encoded by a nucleic acid sequence with which the cell is transformed. The nucleic acid sequence can be an exogenous nucleic acid sequence, a heterologous nucleic acid sequence, or a homologous nucleic acid sequence. The polypeptide can be a full-length yeast epoxide hydrolase or a functional fragment of a full length yeast epoxide hydrolase.

Moreover both processes can be carried out at a pH from 5 to 10. They can be carried out at a temperature of 0° C. to 60° C. In the processes, the concentration of the styrene epoxide can be at least equal to the solubility of the styrene epoxide in water.

In both processes, the styrene epoxide is a compound of the general formula (I) and the vicinal diol (phenylethanediol) produced by the process is a compound of the general formula (II),

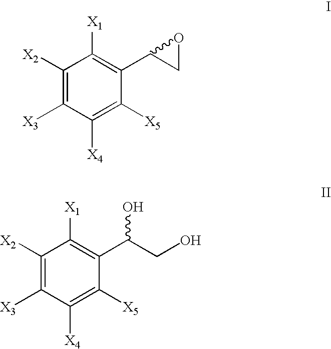

wherein,

X₁, X₂, X₃, X₄ and X₅ are, independently of each other, selected from: H, halogens, hydroxyl groups, mercapto groups, carboxylates, nitro groups, cyano groups, substituted or unsubstituted amino groups, amide groups, alkoxy groups, alkenyloxy groups, aryloxy groups, aryl alkyloxy groups, alkylthio groups, alkoxycarbonyl groups, substituted or unsubstituted carbamoyl groups, acyl groups, substituted and unsubstituted alkyl groups; substituted and unsubstituted alkenyl groups; and substituted and unsubstituted aryl groups, wherein the number of substituents is one or more than one and wherein the substituents are the same or different; or

X₁ and X₂, or X₂ and X₃, or X₃ and X₄, or X₄ and X₅ together and independent are a substituted or unsubstituted aryl group selected from the group consisting of phenyl; biphenyl; naphtyl; anthracenyl groups; and the like; or

X₁ and X₂, or X₂ and X₃, or X₃ and X₄, or X₄ and X₅ together and independent are a cycloalkyl group with 4 to 8 carbon atoms, wherein the cycloalkyl group is selected from the group consisting of cyclobutyl-; cyclopentyl-; cyclohexyl-; cycloheptyl-; and cyclooctyl-groups, wherein the cycloalkyl group is unsubstituted or variably substituted at any position of the ring; or

X₁ and X₂, or X₂ and X₃, or X₃ and X₄, or X₄ and X₅ together and independent are a cycloalkenyl group with 4 to 8 carbon atoms, wherein the cycloalkenyl group is selected from the group consisting of: cyclobutenyl-; cyclopentenyl-; cyclohexenyl-; cycloheptenyl-; and cyclooctenyl-groups, wherein the cycloalkenyl group is unsubstituted or is variably be substituted at one or more positions in the ring; or

X₁ and X₂, or X₂ and X₃, or X₃ and X₄, or X₄ and X₅ together and independent are a heterocyclic group consisting of a 5- to 7-membered heterocyclic group containing a nitrogen atom, an oxygen atom; or a sulfur atom, wherein the heterocyclic group is selected from the group consisting of furyl-; dihydrofuranyl-; tetrahydrofuranyl-; dioxolanyl-; oxazolyl-; dihydrooxazolyl-; oxazolidinyl-; isoxazolyl-; dihydroisoxazolyl-; isoxazolidinyl-; oxathiolanyl-; thienyl-; tetrahydrothienyl-; dithiolanyl-; thiazolyl-; dihydrothiazolyl-; thiazolidinyl-; isothiazolyl-; dihydroisothiazolyl-; isothiazolidinyl-; pyrrolyl-; dihydropyrrolyl-; pyrrolidinyl-; pyrazolyl-; dihydropyrazolyl-; pyrazolidinyl-; imidazolyl-; dihydroimidazolyl-; imidazolidinyl-; triazolyl-; dihydrotriazolyl-; triazolidinyl-; tetrazolyl-; dihydrotetrazolyl-; tetrazolidinyl-; pyridyl-; dihydropyridyl-; piperidinyl-; morpholinyl-; dioxanyl-; oxathianyl-; trioxanyl-; thiomorpholinyl-; pyridazinyl-; dihydropyridazinyl-; tetrahydropyridazinyl-; hexahydropyridazinyl-; pyrimidinyl-; dihydropyrimadinyl-; tetrahydropyrimadinyl-; hexahydropyrimadinyl-; pyrazinyl-; piperazinyl-; pyranyl-; dihydropyranyl-; tetrahydropyranyl-; thiopyranyl-; dihydrothiopyranyl-; tetrahydrothiopyraryl-; dithianyl-; purinyl-; pyrimidinyl-; pyrrolizinyl-; pyrrolizidinyl; indolyl-; dihydroindolyl-; isoindolyl-; indolizinyl-; indolizidinyl-; quinolyl-; dihydroquinolyl-; tetrahydroquinolyl-; isoquinolyl-; dihydroquinolyl-; tetrahydroquinolyl-; quinolizinyl-; quinolizidinyl-; phenanthrolinyl-; chromenyl-; chromanyl-; isochromenyl-; isochromanyl-; benzofuranyl-; and carbazolyl-groups; and the like. The aryl group can be, for example, a substituted or an unsubstituted phenyl group, the cycloalkyl group can be, for example, a cycloalkyl group with 5 to 7 carbon atoms, the cycloalkenyl group can be, for example, a cycloalkenyl group with 5 to 7 carbon atoms, and the heterocyclic group can be, for example, 5 or 6 carbon atoms.

Moreover, in the processes, the enantiomeric mixture can be a racemic mixture or a mixture of any ratio of amounts of the enantiomers. The processes can include adding to the reaction mixture water and at least one water-immiscible solvent, including, for example, toluene, 1,1,2-trichlorotrifluoroethane, methyl tert-butyl ether, methyl isobutyl ketone, dibutyl-o-phtalate, aliphatic alcohols containing 6 to 9 carbon atoms, aliphatic hydrocarbons containing 6 to 16 carbon atoms, or tributyl phosphate. Alternatively, or in addition, the processes can include adding to the reaction mixture water and at least one water-miscible organic solvent, for example, acetone, methanol, ethanol, propanol, isopropanol, acetonitrile, dimethylsulfoxide, N,N-dimethylformamide, or N-methylpyrrolidine. In addition, or alternatively, one or more surfactants, one or more cyclodextrins, or one or more phase-transfer catalysts can be added to the reaction mixtures. Both processes can include stopping the reaction when one enantiomer of the epoxide and/or vicinal diol is in excess compared to the other enantiomer of the epoxide and/or vicinal diol. Furthermore, the processes can include recovering continuously during the reaction the optically active epoxide and/or the optically active vicinal diol produced by the reaction directly from the reaction mixture.

In both processes the yeast cell can be of one of the following exemplary genera: Arxula, Brettanomyces, Bullera, Bulleromyces, Candida, Cryptococcus, Debaryomyces, Dekkera, Exophiala, Geotrichum, Hormon ema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, or Yarrowia

Moreover, in the processes, the yeast cell can be of one of the following exemplary species: Arxula adeninivorans, Arxula terrestris, Brettanomyces bruxellensis, Brettanomyces naardenensis, Brettanomyces anomalus, Brettanomyces species (e.g. NCYC 3151), Bullera dendrophila, Bulleromyces albus, Candida albicans, Candida fabianii, Candida glabrata, Candida haemulonii, Candida intermedia, Candida magnoliae Candida parapsilosis, Candida rugosa, Candida tenuis, Candida tropicalis, Candida famata, Candida kruisei, Candida sp. (new) rel to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Cryptococcus macerans, Debaryomyces hansenii, Dekkera anomala, Exophiala dermatitidis, Geotrichum species (e.g. UOFS Y-0111), Hormonema species (e.g. NCYC 3171), Issatchenkia occidentalis, Kluzyveromyces marxianus, Lipomyces species (e.g. UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotonda glutinis, Rhodotorula minuta, Rhodotorula minuta var. minuta, Rhodotorula mucilaginosa, Rhodotorula philyla, Rhodotorula rubra, Rhodotorula species (e.g. UOFS Y-2042), Rhodotorula species (e.g. UOFS Y-0448), Rhodotorula species (e.g. NCYC 3193), Rhodotorula species (e.g. UOFS Y-0139), Rhodotorula species (e.g. UOFS Y-0560), Rhodotorula aurantiaca, Rhodotorula species (e.g. NCYC 3224), Rhodotorula sp. “mucilaginosa”, Sporidiobolus salmonicol or, Sporobolomyces holsaticus, Sporobolomyces roses, Soboloyces roseus, Sparobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon species (e.g. UOFS Y-0861), Trichosporon species (e.g. UOFS Y-1615), Trichosporon species (e.g. UOFS Y-0451), Trichosporon species (e.g. NCYC 3212), Trichosporon species (e.g. UOFS Y-0449), Trichosporon species (e.g. NCYC 3211), Trichosporon species (e.g. UOFS Y-2113), Trichosporon species (e.g. NCYC 3210), Trichosporon moniliiforme, Trichosporon montevideense, Wingea robertsiae, or Yarrowia lipolytica.

The yeast cell can also be of any of the other genera, species, or strains disclosed herein.

Another aspect of the invention is a method for producing a polypeptide, which process includes the steps of: providing a cell comprising a nucleic acid encoding and capable of expressing a polypeptide that has enantioselective styrene epoxide hydrolase activity; culturing the cell; and recovering the polypeptide from the culture. Recovering the polypeptide from the culture includes, for example, recovering it from the medium in which the cell was cultured or recovering it from the cell per se. The cell can be a yeast cell. The polypeptide can be encoded by an endogenous gene of the cell or the cell can be a recombinant cell, the polypeptide being encoded by a nucleic acid sequence with which the cell is transformed. The nucleic acid sequence can be an exogenous nucleic acid sequence, a heterologous nucleic acid sequence, or a homologous nucleic acid sequence. The polypeptide can be a full-length yeast epoxide hydrolase or a functional fragment of a full-length yeast epoxide hydrolase. The cell can be of any of the yeast genera, species, or strains disclosed herein or any recombinant cell disclosed herein.

The invention also features a crude or pure enzyme preparation which includes an isolated polypeptide having enantioselective styrene epoxide hydrolase activity. The polypeptide can be one encoded by any of the yeast genera, species, or strains disclosed herein or one encoded by a recombinant cell.

In another aspect, the invention features a substantially pure culture of cells, a substantial number of which comprise a nucleic acid encoding, and are capable of expressing, a polypeptide having enantioselective styrene epoxide hydrolase activity. The cells can be recombinant cells or cells of any of the yeast genera, species, or strains disclosed herein.

Another embodiment of the invention is an isolated cell, the cell comprising a nucleic acid encoding a polypeptide having enantioselective styrene epoxide hydrolase activity, the cell being capable of expressing the polypeptide. The cell can be any of those disclosed herein.

The invention also features an isolated DNA that includes: (a) a nucleic acid sequence that encodes a polypeptide that has enantioselective styrene epoxide hydrolase activity and that hybridizes under highly stringent conditions to the complement of a sequence that can be SEQ ID NO: 6, 7, 8, 9, or 10; or (b) the complement of the nucleic acid sequence. The nucleic acid sequence can encode a polypeptide that includes an amino acid sequence that can be SEQ ID NO: 1, 2, 3, 4, or 5. The nucleic acid sequence can be, for example, one of those with SEQ ID NOs: 6, 7, 8, 9, or 10.

Also provided by the invention is an isolated DNA that includes: (a) a nucleic acid sequence that is at least 55% identical to a sequence that can be SEQ ID NO: 6, 7, 8, 9, or 10; or (b) the complement of the nucleic acid sequence, the nucleic acid sequence is encoding a polypeptide that has enantioselective styrene epoxide hydrolase activity.

Another aspect of the invention is an isolated DNA that includes: (a) a nucleic acid sequence that encodes a polypeptide consisting of an amino acid sequence that is at least 55% identical to a sequence that can be SEQ ID NOs: 1, 2, 3, 4, or 5; or (b) the complement of the nucleic acid sequence, the polypeptide having enantioselective styrene epoxide hydrolase activity.

Also included are vectors (e.g., those in which the coding sequence is operably linked to a transcriptional regulatory element) containing any of the above DNAs and cells (e.g., eukaryotic or prokaryotic cells) containing such vectors.

Also provided by the invention is an isolated polypeptide encoded by any of the above DNAs. The polypeptide can include an amino acid sequence that is at least 55% identical to SEQ ID NOs: 1, 2, 3, 4, or 5, the polypeptide having enantioselective styrene epoxide hydrolase activity. The polypeptide can also include: (a) a sequence that can be SEQ ID NO: 1, 2, 3, 4, or 5, or a functional fragment of the sequence; or (b) the sequence of (a), but with no more than five conservative substitutions, the polypeptide having enantioselective styrene epoxide hydrolase activity.

In another embodiment the invention features an isolated antibody (e.g., a polyclonal or a monoclonal antibody) that binds to any of the above-described polypeptides.

The term “exogenous” as used herein with reference to nucleic acid and a particular host cell refers to any nucleic acid that does not occur in (and cannot be obtained from) that particular cell as found in nature. Thus, a non-naturally-occurring nucleic acid is considered to be exogenous to a host cell once introduced into the host cell. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. Nucleic acid that is naturally-occurring can be exogenous to a particular cell. For example, an entire chromosome isolated from a cell of yeast x is an exogenous nucleic acid with respect to a cell of yeast y once that chromosome is introduced into a cell of yeast y.

It will be clear from the above that “exogenous” nucleic acids can be “homologous” or “heterologous” nucleic acids. As used herein, “homologous” nucleic acids are those that are derived from a cell of the same species as the host cell and “heterologous” nucleic acids are those that are derived from a species other than that of the host cell.

In contrast, the term “endogenous” as used herein with reference to nucleic acids or genes and a particular cell refers to any nucleic acid or gene that does occur in (and can be obtained from) that particular cell as found in nature.

The SEQ used and obtained by the methods of the invention can be a compound of the general formula (I) and the vicinal diol produced by the process can be a compound of the general formula (II),

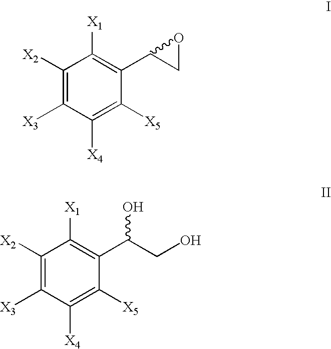

wherein:

X₁, X₂, X₃, X₄, and X₅ can, independently of each other, be selected from: H; halogens (F, Cl, Br, I); hydroxyl groups; mercapto groups; carboxylates; nitro groups; cyano groups; substituted or unsubstituted amino groups (including amino, methylamino, dimethylamino, ethylamino, diethylamino, and various protected amines such as tert-butoxycarbonyl- and arylsulfonamido groups); amide groups (including unsubstituted, N-substituted and N,N′-disubstituted amide groups); alkoxy groups (having 1 to 8 carbon atoms such as methoxy, ethoxy, propyloxy, isopropyloxy, butyloxy, isobutyloxy, tert-butyloxy, pentyloxy, hexyloxy, heptyloxy, or octyloxy); alkenyloxy groups (having: 2 to 8 carbon atoms such as a vinyloxy, allyloxy, 3-butenyloxy or 5-hexenyloxy); aryloxy groups (such as a phenoxy or naphtyloxy group, which can be optionally substituted with an alkyl or alkenyl or alkoxy group having 1 to 4 carbon atoms (or halogens), e.g., phenoxy, 2-methylphenoxy, 3-methylphenoxy, 4-methylphenoxy, 2-allylphenoxy, 2-chlorophenoxy, 3-chlorophenoxy, 4-chlorophenoxy, 4-methoxyphenoxy, 2-allyloxyphenoxy, naphtyloxy, and the like); aryl alkyloxy groups (e.g., benzyloxy and 2-phenylethyloxy); alkylthio groups (having 1 to 8 carbon atoms such as methylthio, ethylthio, propylthio, butylthio, isobutylthio, pentylthio); alkoxycarbonyl groups (e.g., methoxycarbonyl, ethoxycarbonyl, and the like); substituted or unsubstituted carbamoyl group (e.g., carbamoyl, methylcarbamoyl, dimethylcarbamoyl, diethylcarbamoyl and the like); acyl groups (with 1 to 8 carbon atoms such as formyl, acetyl, propionyl or benzoyl groups); substituted and unsubstituted alkyl groups; substituted and unsubstituted alkenyl groups; substituted and unsubstituted aryl groups; and the like.

The number of substituents can be one or more than one.

The substituents can be the same or different.

In another embodiment, X₁ and X₂, or X₂ and X₃, or X₃ and X₄, or X₄ and X₅ together and independently can also form an optionally substituted aryl group selected from the group consisting of: phenyl; biphenyl; naphtyl; anthracenyl groups; and the like. Preferably, the aryl group is a phenyl group, or an optionally substituted phenyl group.

In another embodiment, X₁ and X₂, or X₂ and X₃, or X₃ and X₄, or X₄ and X₅ together and independently can also form a cycloalkenyl group with 4 to 8 carbon atoms. The cycloalkenyl group can be selected from the group consisting of: cyclobutenyl-; cyclopentenyl-; cyclohexenyl-; cycloheptenyl-; and cyclooctenyl-groups that can variably be substituted at any position(s) around the ring. Preferably, the cycloalkenyl group is a cycloalkenyl group with 5 to 7 carbon atoms.

In another embodiment, X₁ and X₂, or X₂ and X₃, or X₃ and X₄, or X₄ and Xs together and independently can also form a heterocyclic group with 5 to 7 atoms in the ring and 1-3 heteroatoms in the ring, where the heteroatom(s) is (are) nitrogen; oxygen or sulfur. Preferably the heterocyclic ring has 5 or 6 atoms. The heterocyclic ring can be selected from the group consisting of; furyl-; dihydrofuranyl-; oxazolyl-; dihydrooxazolyl-; isoxazolyl-; dihydroisoxazolyl-; oxathiolanyl-; thienyl-; dithiolanyl-; thiazolyl-; dihydrothiazolyl-; isothiazolyl-; dihydroisothiazolyl-; pyrrolyl-; dihydropyrrolyl-; pyrazolyl-; dihydropyrazolyl-; imidazolyl-; dihydroimidazolyl-; triazolyl-; dihydrotriazolyl-; tetrazolyl-; dihydrotetrazolyl-; pyridyl-; dihydropyridyl-; pyridazinyl-; dihydropyridazinyl-; tetrahydropyridazinyl-; pyrimidinyl-; dihydropyrimadinyl-; tetrahydropyrimadinyl-; pyrazinyl-; piperazinyl-; pyranyl-; dihydropyranyl-; thiopyranyl-; dihydrothiopyranyl-; dithianyl-; purinyl-; pyrimidinyl-; pyrrolizinyl-; indolyl-; dihydroindolyl-; isoindolyl-; indolizinyl-; indolizidinyl-; quinolyl-; dihydroquinolyl-; isoquinolyl-; dihydroisoquinolyl-; quinolizinyl-; quinolizidinyl-; phenanthrolinyl-; chromenyl-; chromanyl-; isochromenyl-; isochromanyl-; benzofuranyl-; and carbazolyl-groups; and the like.

SEO and phenylethanediol (PED) derivatives can have substituents selected from: halogen styrene epoxides such as 2-, 3- and 4-chlorostyrene epoxide; alkyl styrene epoxides such as 2-, 3- and 4-methyl styrene epoxide; nitro styrene epoxides such as 2-, 3- and 4-nitrostyrene oxide; alkoxy styrene epoxides such as 2-, 3- and 4-methoxystyrene epoxide; 3,4-disubstituted styrene epoxides such as 3,4-dihydroxystyrene epoxide; 3-amido-4-hydroxy styrene epoxide; or 3-NCHO-4-hydroly styrene epoxide.

The styrene epoxide derivatives and phenol ethane diol derivatives can also contain heteroatoms in the ring such as N, O, and S in the 2-, 3- or 4-position, for example, 2-, 3- and 4-pyridyloxiranes.

“Polypeptide” and “protein” are used interchangeably and mean any peptide-linked chain of amino acids, regardless of length or post-translational modification. The invention also features yeast enantioselective styrene epoxide hydrolase (YESH) polypeptides with conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The term “isolated” polypeptide or peptide fragment, as used herein, refers to a polypeptide or a peptide fragment which either has no naturally-occurring counterpart or has been separated or purified from components which naturally accompany it, e.g., microorganism cellular components such as yeast cell cellular components. Typically, the polypeptide or peptide fragment is considered “isolated” when it is at least 70%, by dry weight, free from the proteins and other naturally-occurring organic molecules with which it is naturally associated. Preferably, a preparation of a polypeptide (or peptide fragment thereof) of the invention is at least 80%, more preferably at least 90%, and most preferably at least 99%, by dry weight, the polypeptide (or the peptide fragment thereof), respectively, of the invention. Thus, for example, a preparation of polypeptide x is at least 80%, more preferably at least 90%, and most preferably at least 99%, by dry weight, polypeptide x. Since a polypeptide that is chemically synthesized is, by its nature, separated from the components that naturally accompany it, the synthetic polypeptide is “isolated.”

An isolated polypeptide (or peptide fragment) of the invention can be obtained, for example, by: extraction from a natural source (e.g., from yeast cells); expression of a recombinant nucleic acid encoding the polypeptide; or chemical synthesis. A polypeptide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

An “isolated DNA” is either (1) a DNA that contains sequence not identical to that of any naturally occurring sequence, or (2), in the context of a DNA with a naturally-occurring sequence (e.g., a cDNA or genomic DNA), a DNA free of at least one of the genes that flank the gene containing the DNA of interest in the genome of the organism in which the gene containing the DNA of interest naturally occurs. The term therefore includes a recombinant DNA incorporated into a vector, into an autonomously replicating plasmid or virus, or into the genomic DNA of a prokaryote or eukaryote. The term also includes a separate molecule such as: a cDNA (e.g., SEQ ID NOs: 6, 7, 8, 9, or 10) where the corresponding genomic DNA can include introns and therefore can have a different sequence; a genomic fragment that lacks at least one of the flanking genes; a fragment of cDNA or genomic DNA produced by polymerase chain reaction (PCR) and that lacks at least one of the flanking genes; a restriction fragment that lacks at least one of the flanking genes; a DNA encoding a non-naturally occurring protein such as a fusion protein, mutein, or fragment of a given protein; and a nucleic acid which is a degenerate variant of a cDNA or a naturally occurring nucleic acid. In addition, it includes a recombinant nucleotide sequence that is part of a hybrid gene, i.e., a gene encoding a non-naturally occurring fusion protein. Also included is a recombinant DNA that includes a portion of SEQ ID NOs: 6-10. It will be apparent from the foregoing that isolated DNA does not mean a DNA present among hundreds to millions of other DNA molecules within, for example, cDNA or genomic DNA libraries or genomic DNA restriction digests in, for example, a restriction digest reaction mixture or an electrophoretic gel slice.

As used herein, a “functional fragment” of a YESH polypeptide is a fragment of the polypeptide that is shorter than the full-length polypeptide and has at least 20% (e.g., at least: 30%; 40%; 50%; 60%; 70%; 80%; 90%; 95%; 98%; 99%; 100%, or more) of the ability of the full-length polypeptide to enantioselectively hydrolyse a SEQ of interest. Fragments of interest can be made by either recombinant, synthetic, or proteolytic digestive methods and tested for their ability to enantioselectively hydrolyse a SEQ.

As used herein, “operably linked” means incorporated into a genetic construct so that an expression control sequence effectively controls expression of a coding sequence of interest.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., styrene epoxides (SEQ) and phenylethanediols (PED) substantially enriched for one optical enantiomer, will be apparent from the following description, from the drawings and from the claims.

### DESCRIPTION OF DRAWINGS

FIG. 1 is a restriction map of the pYLHmA (pINA1291) expression vector. The positions of the hpd4 promoter and LIP2 terminator and of unique restriction sites available for the insertion of coding sequences are indicated.

FIG. 2 is a restriction map of the pYLTsA pKOV93 expression vector. The positions of the TEF promoter and LIP2 terminator and of unique restriction sites available for the insertion of coding sequences are indicated.

FIGS. 3-7 (Samples 24-28) are line graphs showing the hydrolysis of (±)-unsubstituted SEQ by the indicated wild type yeast strains cultivated in shake flasks to produce optically active (S)-unsubstituted SEQ and (R)-unsubstituted phenylethanediol (PED). The left y-axis (and lines with shaded circle and triangle data points) in each graph show the changes in concentrations of the epoxides at the time points indicated on the x-axis and the right y-axis (and lines with shaded square and diamond-shaped data points) in each graph show the enantiomeric excesses (“ee”) and degree of conversion (% epoxide converted to diol as a % of the total (both enantiomers) starting epoxide concentration) at the time points indicated on the x-axis. The biocatalyst loadings are indicated in brackets beside the strain names in each figure. The percentage biocatalyst loading refers to a percentage wet weight of yeast cells or an equivalent wet weight of lysed/broken yeast cells in the aqueous fraction of the reaction matrix. In all these experiments, the value of the percentage wet weight of biocatalyst is approximately five-fold the value of the equivalent dry weight of biocatalyst. The biocatalyst loadings are indicated in a similar fashion in all the biotransformation reaction profiles shown in the figures below.

FIG. 8 (Samples 29) is a line graph showing the hydrolysis of (±)-unsubstituted SEQ by the indicated wild type yeast strain cultivated in shake flasks to produce optically active (S)-unsubstituted SEQ and (R)-unsubstituted phenylethanediol (PED). The lines with shaded circle and shaded triangle data points show the changes in concentrations of the epoxides at the time points indicated on the x-axis. The lines with open circle and open triangle data points show the changes in concentrations of the diols.

FIG. 9 (Sample 30) is a line graph showing the hydrolysis of (±)-unsubstituted SEQ by a wild type yeast strain cultivated in a 10 L volume in a 15 L fermenter (bioreactor) to produce optically active (S)-unsubstituted SEQ and (R)-unsubstituted PED. The left y-axis (and lines with shaded circle and triangle data points) in each graph show the changes in concentrations of the epoxides at the time points indicated on the x-axis and the right y-axis (and lines with shaded square and diamond-shaped data points) in each graph show the enantiomeric excesses (“ee”) and degree of conversion (% epoxide converted to diol as a % of the starting epoxide concentration) at the time points indicated on the x-axis.

FIGS. 10-12 (Samples 31-33) are line graphs showing the hydrolysis of (±)-unsubstituted SEQ to produce optically active (S)-unsubstituted SEQ and (R)-unsubstituted PED by yeast host strains transformed with vectors expressing YESH polypeptides from selected wild type yeast strains. The left y-axis (and lines with shaded circle and triangle data points) in each graph show the changes in concentrations of the epoxides at the time points indicated on the x-axis and the right y-axis (and lines with shaded square and diamond-shaped data points) in each graph show the enantiomeric excesses (“ee”) and conversions at the time points indicated on the x-axis.

FIGS. 13-16 (Samples 34-37) are line graphs showing the hydrolysis of (f)-unsubstituted SEQ by either whole or lysed (“Y-PER treated”) cells of yeast host strains transformed with either multi-copy or with single-copy integrative vectors expressing YESH polypeptides from the yeast strain Rhodotorula araucariae (NCYC 3183) to produce optically active (S)-unsubstituted SEQ and (R)-unsubstituted PED. The data is presented as concentrations of both enantiomers of the epoxide at the indicated time points.

FIG. 17 (Sample 38) is a line graph showing the improved performance in hydrolysis of (±)-unsubstituted SEQ to produce optically active (S)-unsubstituted SEQ and (R)-unsubstituted PED by a host strain transformed with a vector expressing a YESH polypeptide from a selected wild type yeast strain Rhodotorula araucariae (NCYC 3183) cultivated in a 15 L fermenter to produce optically active (S)-unsubstituted SEQ and (R)-unsubstituted PED. Biotransformation in a stirred tank reactor using low temperature, tri-butyl phosphate additive and high epoxide concentrations further increased the efficiency of the reaction. The left y-axis (and lines with shaded circle and triangle data points) shows the changes in concentrations of the epoxides at the time points indicated on the x-axis and the right y-axis (and lines with shaded square and diamond-shaped data points) shows the enantiomeric excesses (“ee”) and conversions at the time points indicated on the x-axis.

FIG. 18 (Scheme 1) is a schematic representation of the hydrolysis of two exemplary substituted styrene epoxides (SEQ) illustrating the enantioselective SEQ hydrolysis.

FIGS. 19-23 (Samples 133-137) are line graphs showing the hydrolysis of (±)-3-chloroSEO by the indicated wild type yeast strains to produce optically active (S)-3-chloroSEO and (R)-3-chloroPED. The A panel in each figure is a line graph showing the change in concentrations of the epoxide enantiomers with time and the B panel in each figure is a line graph showing the enantiomeric excess of the (S)-epoxide at different conversions.

FIGS. 24-28 (Samples 138-142) are line graphs showing the hydrolysis of (±)-3-chloroSEO to produce optically active (S)-3-chloroSEO and (R)-3-chloroPED by various yeast strains transformed with vectors expressing YESH polypeptides from selected wild type yeast strains. The A panel in each figure is a line graph showing the change in concentrations of the epoxide enantiomers with time and the B panel in each figure is a line graph showing the enantiomeric excess of the (S)-epoxide at different conversions.

FIGS. 29 and 30 (Samples 143-144) are line graphs showing the hydrolysis of (±)-2-chloroSEO to produce optically active (S)-2-chloroSEO and (R)-2-chloroPED by two yeast strains transformed with vectors expressing YESH polypeptides from selected wild type yeast strains. The A panel in each figure is a line graph showing the change in concentrations of the epoxide enantiomers with time and the B panel in each figure is a line graph showing the enantiomeric excess of the epoxide at different conversions.

FIGS. 31-36 (Samples 145-150) are line graphs showing the hydrolysis of (−)-4-chloroSEO to produce optically active (S)-4-chloroSEO and (R)-4-chloroPED by various t yeast strains transformed with vectors expressing YESH polypeptides from selected wild type yeast strains. The A panel in each figure is a line graph showing the change in concentrations of the epoxide enantiomers with time and the B panel in each figure is a line graph showing the enantiomeric excess of the (S)-epoxide at different conversions.

FIGS. 37-44 (Samples 151-158) are line graphs showing the hydrolysis of (±)-2, 3-, or -4-nitroSEO to produce optically active (S) or (R)-2,3-, or 4-nitroSEO (and associated nitroPED) by a wild-type yeast strain (FIG. 37) and various t yeast host strains (FIGS. 38-44) transformed with vectors expressing YESH polypeptides from selected wild type yeast strains. The A panel in each figure is a line graph showing the change in concentrations of the epoxide enantiomers with time and the B panel in each figure is a line graph showing the enantiomeric excess of the excess epoxide enantiomer at different conversions.

FIG. 45 is a depiction of the amino acid sequence (SEQ ID NO:1) of a YESH polypeptide encoded by cDNA derived from a Rhodosporidium toruloides strain (assigned accession no. NCYC 3181).

FIG. 46 is a depiction of the amino acid sequence (SEQ ID NO:2) of a YESH polypeptide encoded by cDNA derived from a Rhodosporidium toruloides strain (assigned identification no. UOFS Y-0471).

FIG. 47 is a depiction of the amino acid sequence (SEQ ID NO:3) of a YESH polypeptide encoded by cDNA derived from a Rhodotorula araucariae strain (assigned accession no. NCYC 3183).

FIG. 48 is a depiction of the amino acid sequence (SEQ ID NO:4) of a YESH polypeptide encoded by cDNA derived from a Rhodosporidium paludigenum strain (assigned accession no. NCYC 3179).

FIG. 49 is a depiction of the amino acid sequence (SEQ ID NO:5) of a YESH polypeptide encoded by a cDNA derived from a Rhodotorula mucilaginosa strain (assigned accession no. NCYC 3190).

FIG. 50 is a depiction of the nucleotide sequence (SEQ ID NO:6) of a YESH polypeptide encoded by cDNA derived from a Rhodosporidium toruloides strain (assigned accession no. NCYC 3181).

FIG. 51 is a depiction of the nucleotide sequence (SEQ ID NO:7) of a YESH polypeptide-encoding cDNA derived from a Rhodosporidium toruloides strain (assigned identification no. UOFS Y-0471

FIG. 52 is a depiction of the nucleotide sequence (SEQ ID NO:8) of a YESH polypeptide-encoding cDNA derived from a Rhodotorula araucariae strain (assigned accession no. NCYC 3183).

FIG. 53 is a depiction of the nucleotide sequence (SEQ ID NO:9 of a YESH polypeptide-encoding cDNA derived from a Rhodosporidium paludigenum strain (assigned accession no. NCYC 3179).

FIG. 54 is a depiction of the nucleic acid sequence (SEQ ID NO:10) of a YESH polypeptide-encoding cDNA derived from a Rhodotorula mucilaginosa strain (assigned accession no. NCYC 3190).

FIG. 55 is a table showing the homology at the amino acid level of the YESH polypeptides with SEQ ID NOs: 1-5.

FIG. 56 is a table showing the homology at the nucleotide level of YESH-encoding cDNA molecules with SEQ ID NOs: 6-10.

FIG. 57 is a depiction of the amino acid sequences of eight enantioselective epoxide hydrolases aligned for maximum homology. Also shown are consensus amino acids. The sequences labeled #1, #46, #25, #692 and #23 correspond to SEQ ID NOs: 1-5 and the sequences labeled Car054 (SEQ ID NO. 27), Jen46-2 (SEQ ID NO. 28), and #777 (SEQ ID NO. 29) correspond to enantioselective hydrolases catalyzing the hydrolysis of non-SOE epoxides. The consensus catalytic triad is composed of a nucleophile, an acid and a base, the positions of which are indicated by N, A and B, respectively. “HGXP” represents the region of the oxy-anion hole of the enzymes. “sxNxss” represents the genetic motif found in α/β-hydrolase fold enzymes. (P=homology; P=identity of 75-100%; P=identity of 50-75%; .=gap).

### DETAILED DESCRIPTION

Various aspects of the invention are described below.

### Nucleic Acid Molecules

The YESH nucleic acid molecules of the invention can be cDNA, genomic DNA, synthetic DNA, or RNA, and can be double-stranded or single-stranded (i.e., either a sense or an antisense strand). Segments of these molecules are also considered within the scope of the invention, and can be produced by, for example, the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide (for example, one of the polypeptides with SEQ ID NOS: 1-5). In addition, these nucleic acid molecules are not limited to coding sequences, e.g., they can include some or all of the non-coding sequences that lie upstream or downstream from a coding sequence.

The nucleic acid molecules of the invention can be synthesized (for example, by phosphoramidite-based synthesis) or obtained from a biological cell, such as the cell of a eukaryote (e.g., a mammal such as human or a mouse or a yeast such as any of the genera, species, and strains of yeast disclosed herein) or a prokaryote (e.g., a bacterium such as Escherichia coli). The nucleic acids can be those of a yeast such as any of the genera, species, and strains of yeast disclosed herein. Combinations or modifications of the nucleotides within these types of nucleic acids are also encompassed.

In addition, the isolated nucleic acid molecules of the invention encompass segments that are not found as such in the natural state. Thus, the invention encompasses recombinant nucleic acid molecules (for example, isolated nucleic acid molecules encoding the polypeptides of SEQ ID NOs: 1-5) incorporated into a vector (for example, a plasmid or viral vector) or into the genome of a heterologous cell (or the genome of a homologous cell, at a position other than the natural chromosomal location). Recombinant nucleic acid molecules and uses therefor are discussed further below.

Techniques associated with detection or regulation of genes are well known to skilled artisans. Such techniques can be used, for example, to test for expression of a YESH gene in a test cell (e.g., a yeast cell) of interest.

A YESH family gene or protein can be identified based on its similarity to the relevant YESH gene or protein, respectively. For example, the identification can be based on sequence identity. The invention features isolated nucleic acid molecules which are, or are at least 50% (e.g., at least: 55%; 60%; 65%; 75%; 85%; 95%; 98%; or 99%) identical to: (a) a nucleic acid molecule that encodes the polypeptide of SEQ ID NOs: 1-5; (b) the nucleotide sequence of SEQ ID NOs: 6-10; (c) a nucleic acid molecule which includes a segment of at least 15 (e.g., at least: 20; 25; 30; 35; 40; 50; 60; 80; 100; 125; 150; 175; 200; 250; 300; 350; 400; 500; 600; 700; 800; 900; 1,000; 1,100; 1,150; 1,160; 1,170; 1,175; 1,178; 1,180; 1,181; 1,200; 1,220; 1,225; 1,226; 1,228; 1,230; 1,231; or 1,232) nucleotides of SEQ ID NOs: 6-10; (d) a nucleic acid molecule encoding any of the polypeptides or fragments thereof disclosed below; and (e) the complement of any of the above nucleic acid molecules. The complements of the above molecules can be full-length complements or segment complements containing a segment of at least 15 (e.g., at least: 20; 25; 30; 35; 40; 50; 60; 80; 100; 125; 150; 175; 200; 250; 300; 350; 400; 500; 600; 700; 800; 900; 1,000; 1,100; 1,200; 1,220; 1,225; 1,228; 1,230; 1,231; or 1,232) consecutive nucleotides complementary to any of the above nucleic acid molecules. Identity can be over the full-length of SEQ ID NOs: 6-10 or over one or more contiguous or non-contiguous segments.

The determination of percent identity between two sequences is accomplished using the mathematical algorithm of Karlin and Altschul, Proc. Natl. Acad. Sci. USA 90, 5873-5877, 1993. Such an algorithm is incorporated into the BLASTN and BLASTP programs of Altschul et al. (1990). J. Mol. Biol. 215, 403-410. BLAST nucleotide searches are performed with the BLASTN program, score=100, wordlength=12, to obtain nucleotide sequences homologous to HIN-1-encoding nucleic acids. BLAST protein searches axe performed with the BLASTP program, score=50, wordlength=3, to obtain amino acid sequences homologous to the HIN-1 polypeptide. To obtain gap alignments for comparative purposes, Gap BLAST is utilized as described in Altschul et al. (1997) Nucleic Acids Res. 25, 3389-3402. When utilizing BLAST and Gap BLAST programs, the default parameters of the respective programs (e.g., XBLAST and NBLAST) are used.

Hybridization can also be used as a measure of homology between two nucleic acid sequences. A YESH-encoding nucleic acid sequence, or a portion thereof; can be used as a hybridization probe according to standard hybridization techniques. The hybridization of a YESH probe to DNA or RNA from a test source (e.g., a mammalian cell) is an indication of the presence of YESH DNA or RNA in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6×sodium chloride/sodium citrate (SSC) at 45° C., followed by a wash in 0.2×SSC, 0.1% SDS at 65° C.

The invention also encompasses: (a) vectors (see below) that contain any of the foregoing YESH coding sequences (including coding sequence segments) and/or their complements (that is, “antisense” sequences); (b) expression vectors that contain any of the foregoing YESH coding sequences (including coding sequence segments) operably linked to one or more transcriptional and/or translational regulatory elements (TRE; examples of which are given below) necessary to direct expression of the coding sequences; (c) expression vectors encoding, in addition to a YESH polypeptide (or a fragment thereof), a sequence unrelated to YESH, such as a reporter, a marker, or a signal peptide fused to YESH; and (d) genetically engineered host cells (see below) that contain any of the foregoing expression vectors and thereby express the nucleic acid molecules of the invention.

Recombinant nucleic acid molecules can contain a sequence encoding a YESH polypeptide or a YESH polypeptide having an heterologous signal sequence. The full length YESH polypeptide, or a fragment thereof, can be fused to such heterologous signal is sequences or to additional polypeptides, as described below. Similarly, the nucleic acid molecules of the invention can encode a YESH that includes an exogenous polypeptide that facilitates secretion.

The TRE referred to above and further described below include but are not limited to inducible and non-inducible promoters, enhancers, operators and other elements that are known to those skilled in the art and that drive or otherwise regulate gene expression.

Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors. Other useful TRE are listed in the examples below.

Similarly, the nucleic acid can form part of a hybrid gene encoding additional polypeptide sequences, for example, a sequence that functions as a marker or reporter. Examples of marker and reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), xanthine guanine phosphoribosyltransferase (XGPRT), and green, yellow, or blue, fluorescent protein. As with many of the standard procedures associated with the practice of the invention, skilled artisans will be aware of additional useful reagents, for example, additional sequences that can serve the function of a marker or reporter. Generally, the hybrid polypeptide will include a first portion and a second portion; the first portion being a YESH polypeptide (or any of YESH fragments described below) and the second portion being, for example, the reporter described above or an Ig heavy chain constant region or part of an Ig heavy chain constant region, e.g., the CH2 and CH3 domains of IgG2a heavy chain. Other hybrids could include an antigenic tag or a poly-His tag to facilitate purification.

The expression systems that can be used for purposes of the invention include, but are not limited to, microorganisms such as yeasts (e.g., any of the genera, species or strains listed herein) or bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces, Kluyveromyces, Hansenula, Pichia, Yarrowia, Arxula and Candida, and other genera, species, and strains listed herein) transformed with recombinant yeast expression vectors containing the nucleic acid molecule of the invention; insect cell systems infected with recombinant virus expression vectors (for example, baculovirus) containing the nucleic acid molecule of the invention; plant cell systems infected with recombinant virus expression vectors (for example, cauliflower mosaic virus (CaMV) or tobacco mosaic virus (TMV)) or transformed with recombinant plasmid expression vectors (for example, Ti plasmid) containing a YESH nucleotide sequence; or mammalian cell systems (for example, COS, CHO, BHK, 293, VERO, HeLa, MDCK, W138, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal and transfected with a plasmid vector or infected with a viral vector.

The invention includes wild-type and recombinant cells including, but not limited to, yeast cells (e.g., any of those disclosed herein) containing any of the above YESH genes, nucleic acid molecules, and genetic constructs. Other cells that can be used as host cells are listed herein. The cells are preferably isolated cells. As used herein, the term “isolated” as applied to a microorganism (e.g., a yeast cell) refers to a microorganism which either has no naturally-occurring counterpart (e.g., a recombinant microorganism such as a recombinant yeast) or has been extracted and/or purified from an environment in which it naturally occurs. Thus, an “isolated microorganism” does not include one residing in an environment in which it naturally occurs, for example, in the air, outer space, the ground, oceans, lakes, rivers, and streams and the like, ground at the bottom of oceans, lakes, rivers, and streams and the like, snow, ice on top of the ground or in/on oceans lakes, rivers, and streams and the like, man-made structures (e.g., buildings), or in natural hosts (e.g., plant, animal or microbial hosts) of the microorganism, unless the microorganism (or a progenitor of the microorganism) was previously extracted and/or purified from an environment in which it naturally occurs and subsequently returned to such an environment or any other environment in which it can survive. An example of an isolated microorganism is one in a substantially pure culture of the microorganism.

Moreover the invention provides a substantially pure culture of a microorganism (e.g., a microbial cell such as a yeast cell). As used herein, a “substantially pure culture” of a microorganism is a culture of that microorganism in which less than about 40% (i.e., less than about: 35%; 30%; 25%; 20%; 15%; 10%; 5%; 2%; 1%; 0.5%; 0.25%; 0.1%; 0.01%; 0.001%; 0.0001%; or even less) of the total number of viable microbial (e.g., bacterial, fungal (including yeast), mycoplasmal, or protozoan) cells in the culture are viable microbial cells other than the microorganism. The term “about” in this context means that the relevant percentage can be 15% percent of the specified percentage above or below the specified percentage. Thus, for example, about 20% can be 17% to 23%. Such a culture of microorganisms includes the microorganisms and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

The microbial cells of the invention can be stored, for example, as frozen cell suspensions, e.g., in buffer containing a cryoprotectant such as glycerol or sucrose, as lyophilized cells. Alternatively, they can be stored, for example, as dried cell preparations obtained, e.g., by fluidised bed drying or spray drying, or any other suitable drying method. Similarly the enzyme preparations can be frozen, lyophilised, or immobilized and stored under appropriate conditions to retain activity.

### Polypeptides and Polypeptide Fragments

The YESH polypeptides of the invention include all the YESH and fragments of YESH disclosed herein. They can be, for example, the polypeptides with SEQ ID NOs: 1-5 and functional fragments of these polypeptides. The polypeptides embraced by the invention also include fusion proteins that contain either full-length or a functional fragment of it fused to unrelated amino acid sequence. The unrelated sequences can be additional functional domains or signal peptides.

The invention features isolated polypeptides which are, or are at least 50% (e.g., at least: 55%; 60%; 65%; 75%; 85%; 95%; 98%; or 99%) identical to the polypeptides with SEQ ID NOs: 1-5. The identity can be over the full-length of the latter polypeptides or over one or more contiguous or non-contiguous segments.

Fragments of YESH polypeptide are segments of the full-length YESH polypeptide that are shorter than full-length YESH. Fragments of YESH can contain 5-410 (e.g., 10, 20, 30, 40, 50, 60, 70, 80, 90, 100, 120, 150, 250, 300, 350, 380, 390, 391, 392, 393, 400, 405, 406, 407, 408, 409, or 410) amino acids of SEQ ID NOs:1-5. Fragments of YESH can be functional fragments or antigenic fragments.

The polypeptides can be any of those described above but with not more 50 (e.g., not more than 50, 45, 40, 35, 30, 25, 20, 17, 14, 12, 10, nine, eight, seven, six, five, four, three, two, or one) conservative substitution(s). Such substitutions can be made by, for example, site-directed mutagenesis or random mutagenesis of appropriate YESH coding sequences

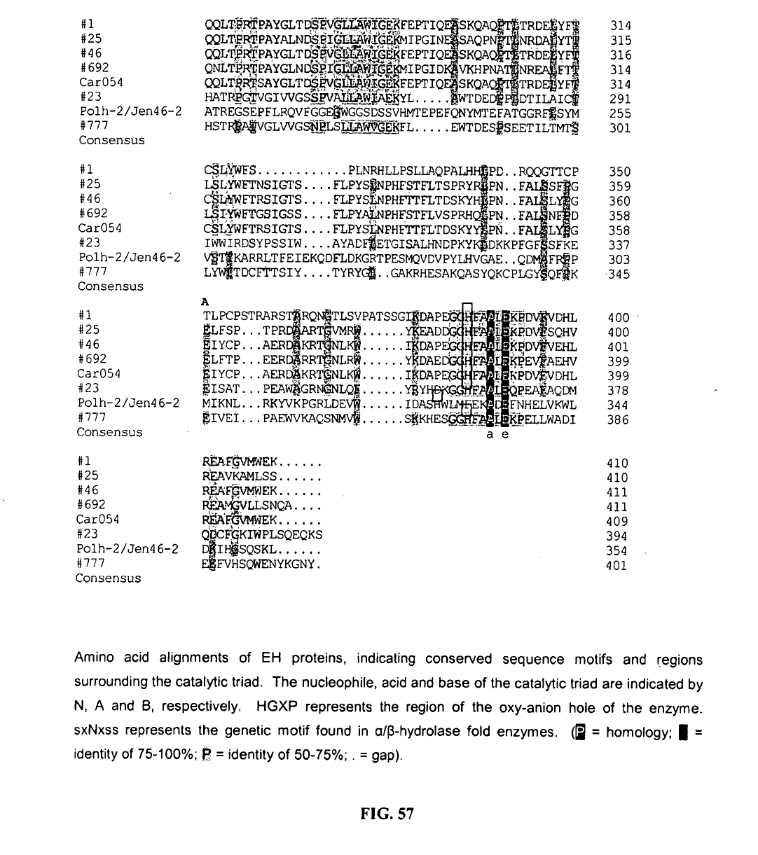

“Functional fragments” of a YESH polypeptide (and, optionally, any of the above-described YESH polypeptide variants) have at least 20% (e.g., 30%, 40%, 50%, 60%, 70%, 80%, 90%, 95%, 98%, 99%, 100%, or more) of the ability of the full-length, wild-type YESH polypeptide to enantioselectively hydrolyse a SEQ of interest. One of skill in the art will be able to predict YESH functional fragments using his or her own knowledge and information provided herein, e.g., the amino acid alignments in FIG. 57 showing highly conserved domains and residues required for epoxide hydrolase activity.

Fragments of interest can be made either by recombinant, synthetic, or proteolytic digestive methods and tested for their ability to enantioselectively hydrolyse a SEQ.

Antigenic fragments of the polypeptides of the invention are fragments that can bind to an antibody. Methods of testing whether a fragment of interest can bind to an antibody are known in the art.

The polypeptides can be purified from natural sources (e.g., wild-type or recombinant yeast cells such as any of those described herein). Smaller peptides (e.g., is those less than about 100 amino acids in length) can also be conveniently synthesized by standard chemical means. In addition, both polypeptides and peptides can be produced by standard in vitro recombinant DNA techniques and in vivo transgenesis, using nucleotide sequences encoding the appropriate polypeptides or peptides. Methods well-known to those skilled in the art can be used to construct expression vectors containing relevant coding sequences and appropriate transcriptional/translational control signals. See, for example, the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology [Green Publishing Associates and Wiley Interscience, N.Y., 1989].

Polypeptides and fragments of the invention also include those described above, but modified by the addition, at the amino- and/or carboxyl-terminal ends, of a blocking agent to facilitate survival of the relevant polypeptide. This can be useful in those situations in which the peptide termini tend to be degraded by proteases. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or non-covalently coupled to pharmaceutically acceptable “carrier” proteins prior to administration.

Also of interest are peptidomimetic compounds that are designed based upon the amino acid sequences of the functional peptide fragments. Peptidomimetic compounds are synthetic compounds having a three-dimensional conformation (i.e., a “peptide motif”) that is substantially the same as the three-dimensional conformation of a selected peptide. The peptide motif provides the peptidomimetic compound with the ability to enantioselectively hydrolyse a SEQ of interest in a manner qualitatively identical to that of the YESH functional fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

The invention also provides compositions and preparations containing one or more (e.g., two, three, four, five, six, seven, eight, nine, ten, 12, 15, 20, 25, or more) of the above-described polypeptides, polypeptide variants, and polypeptide fragments. The composition or preparation can be, for example a crude cell (e.g., yeast cell) extract or culture supernatant, a crude enzyme preparation, a highly purified enzyme preparation. The compositions and preparations can also contain one or more of a variety of carriers or stabilizers known in the art. Carriers and stabilizers are known in the art and include, for example: buffers, such as phosphate, citrate, and other non-organic acids; antioxidants such as ascorbic acid; low molecular weight (less than 10 residues) polypeptides; proteins such as serum albumin, gelatin or immunoglobulins; hydrophilic polymers such polyvinylpyrrolidone; amino acids such as glycine, glutamine, asparagine, arginine, or lysine; monosaccharides, disaccharides, and other carbohydrates including glucose, mannose, or dextrans; chelating agents such as ethylenediaminetetraacetic acid (EDTA); sugar alcohols such as mannitol, or sorbitol; salt-forming counterions such as sodium; and/or nonionic surfactants such as Tween and Pluronics.

### Methods of Producing Optically Active Epoxides and Optically Active Vicinal Diols

The invention provides methods for obtaining enantiopure, or substantially enantiopure, optically active SEQ and optically active PED. Enantiopure optically active SEQ or PED preparations are preparations containing one enantiomer of the SEQ or FED and none of the other enantiomer of the SEQ or PED. “Substantially enantiopure” optically active SEQ or PED preparations are preparations containing at least 55% (e.g., at least: 60%; 70%; 80%; 85%; 90%; 95%; 97%; 98%; 99%; 99.5%; 99.8%; or 99.9%), relative to the total amount of both SEQ or FED enantiomers, of the particular enantiomer of the SEQ or the PED.

The method involves exposing a SEQ sample containing a mixture of both enantiomers of the SEQ to a YESH polypeptide (e.g., an isolated YESH polypeptide or one in a microbial cell), which selectively catalyzes the conversion of one of the enantiomers of the SEQ to a corresponding PED. In this way the desired PED is produced, the selective SEQ enantiomer substrate for the YESH is selectively depleted, and the relative proportion (of the total amount of the SEQ) of the other SEQ enantiomer is increased. YESH polypeptides useful for the invention (i.e., those with SEQ enantioselective activity) will catalyze the conversion of one enantiomer of a SEQ to its corresponding PED with less than 80% (e.g., less than: 70%, 60%, 50%, 40%, 30%; 20%; 10%; 5%; 2.5%; 1%; 0.5%; 0.01%) of the efficiency that its catalyzes the conversion of the other enantiomer of the SEQ to its corresponding PED. The starting enantiomeric mixtures can be racemic with respect to the two SEQ enantiomers or they can contain various proportions of the two SEQ enantiomers ((e.g., 95:5, 90:10, 80:20, 70:30, 60:40 or 50:50) In addition, optimal concentrations of the SEQ and conditions of incubation will vary from one YESH polypeptide to another and from one SEQ to another. Given the teachings of the working examples contained herein, one skilled in the art will know how to select working conditions for the production of a desired enantiomer of a desired PED and/or SEQ.

The method can be implemented by, for example, incubating (culturing) the SEQ enantiomeric mixtures with a wild-type yeast cell or a recombinant cell (yeast or any other host species listed herein) containing a nucleic acid sequence (e.g., a gene or a recombinant nucleic acid sequence) encoding a YESH polypeptide, a crude extract from such cells, a semi-purified preparation of a YESH polypeptide, or an isolated YESH polypeptide, all of which exhibit epoxide hydrolase activity with chiral preference.

The strain of the yeast cell may be selected from the following genera: Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, or Yarrowia.

Yeast strains innately capable of producing a polypeptide that converts or hydrolyses mixtures of SEQ to optically active (i.e. enantiopure or substantially enantiopure) SEQ and/or PED include the following exemplary genera and species:

| Genus | Species |
|---|---|
| Bullera | B. dendrophila |
| Candida | C. magnoliae |
| | C. rugosa |
| Cryptococcus | C. albidus |
| | C. curvatus |
| | C. hungaricus |
| | C. laurentii |
| | C. podzolicus |
| Debaryomyces | D. hansenii |
| Pichia | P. finlandica |
| | P. guillermondii |
| | P. haplophila |
| Rhodosporidium | R. paludigenum |
| | R. toruloides |
| Rhodotorula | R. aurantiaca |
| | R. araucariae |
| | R. aurantiaca |
| | R. glutinis |
| | R. minuta |
| | R. mucilaginosa |
| | R. species (e.g., UOFS Y-2043) |
| Sporidiobolus | S. salmonicolor |
| Sporobolomyces | S. holsaticus |
| | S. roseus |
| | S. tsugae |
| Trichosporon | T. cataneum var. cutaneum |
| | T. montevideense |
| | T. ovoides |
| | T. species (e.g. UOFS Y-0533) |
| Yarrowia | Y. lipolytica |

The yeast strain can be, for example, one selected from Tables 2 or 3 (see below).

Cultivation in bioreactors (fermenters) of yeast strains expressing a YESH polypeptide, or fragment thereof (with the purpose of preparing yeasts stocks or for the enantioselective preparative methods of the invention) can be carried out under conditions that provide useful biomass and/or enzyme titer yields. Cultivation can be by batch, fed-batch or continuous culture methods. Useful cultivation conditions are dependent on the yeast strain used. General procedures for establishing useful growth conditions of yeasts, fungi and bacteria in bioreactors are known to those skilled in the art. The mixture of epoxides can be added directly to the culture. The concentration of the SEQ enantiomeric mixture in the reaction matrix can be at least equal to the soluble concentration of the SEQ enantiomeric mixture in water. The preferred epoxide level in the reaction matrix is greater than the solubility limit in the aqueous reaction medium thereby resulting in a two phase reaction system. The starting amount of epoxide added to the reaction mixture is not critical, provided that the concentration is at least equal to the solubility of the specific epoxide in the aqueous reaction medium. The epoxide can be metered out continuously or in batch mode to the reaction mixture. The relative proportions of (R)- and (S)-epoxide in the mixture of enantiomers of the epoxide shown by the general formula (I) is not critical but it is advantageous for commercial purpose to employ a racemic form of the epoxide shown by the general formula (I). The epoxide can be added in a racemic form or as a mixture of enantiomers in different ratios.

The amount of the yeast cells, crude yeast cell extract, or partially purified or isolated polypeptide having SEQ enantioselective activity added to the reaction depends on the kinetic parameters of the specific reaction and the amount of epoxide that is to be hydrolysed. In the case of product inhibition, it can be advantageous to remove the formed vicinal diol from the reaction mixture or to maintain the concentration of the vicinal diol at levels that allow reasonable reaction rates. Techniques used to enhance enzyme and biomass yields include the identification of useful (or optimal) carbon sources, nitrogen sources, cultivation time, dilution rates (in the case of continuous culture) and feed rates, carbon starvation, addition of trace elements and growth factors to the culture medium, and addition of inducers for example substrates or substrate analogs of the epoxide hydrolases during cultivation. In the case of recombinant hosts, the conditions under which the promoters function workably for transcription of the gene encoding the polypeptide with epoxide hydrolase activity are taken into account. At the end of fermentation (culture), biomass and culture medium can be separated by methods known to one skilled in the art, such as filtration or centrifugation.

The processes are generally performed under mild conditions. For example, the reactions can be carried out at a pH from 5 to 10, preferably from 6.5 to 9, and most preferably from 7 to 8.5. The temperature for hydrolysis can be from 0 to 70° C., preferably from 0 to 50° C., most preferably from 4 to 40° C. It is also known that lowering of the temperature of the reaction can enhance enantioselectivity of an enzyme.

The reaction mixture can contain mixtures of water with at least one water-miscible solvents (e.g., water-miscible organic solvents). Preferably, water-miscible solvents are added to the reaction mixture such that epoxide hydrolase activity remains measurable. Water-miscible solvents are preferably organic solvents and can be, for example, acetone, methanol, ethanol, propanol, isopropanol, acetonitrile, dimethylsulfoxide, N,N-dimethylformamide, N-methylpyrrolidine, and the like.

The reaction mixture can also, or alternatively, contain mixtures of water with at least one water-immiscible organic solvent. Examples of water-immiscible solvents that can be used include, for example, toluene, 1,1,2-trichlorotrifluoroethane, methyl tert-butyl ether, methyl isobutyl ketone, dibutyl-o-phtalate, aliphatic alcohols containing 6 to 9 carbon atoms (for example hexanol, octanol), aliphatic hydrocarbons containing 6 to 16 carbon atoms (for example cyclohexane, n-hexane, n-octane, n-decane, n-dodecane, n-tetradecane and n-hexadecane or mixtures of the aforementioned hydrocarbons), and the like. Thus, the reaction mixture can include water with at least one water-immiscible organic solvent selected from the group consisting of toluene, 1,1,2-trichlorotrifluoroethane, methyl tert-butyl ether, methyl isobutyl ketone, dibutyl-o-phtalate, aliphatic alcohols containing 6 to 9 carbon atoms, and aliphatic hydrocarbons containing 6 to 16 carbon.

The reaction mixture can also contain surfactants (for example, Tween 80), cyclodextrins or any agent that can increase the solubility, selectively or otherwise, of the epoxide enantiomers in the aqueous reaction phase.

The reaction mixture can also contain a buffer. Buffers are known in the art and include, for example, phosphate buffers, Tris buffer, and HEPES buffers.

The production of the YESH polypeptides, including functional fragments, can be, for example, as recited above in the section on Polypeptides and Polypeptide Fragments. Thus they can made by production in a natural host cell, production in a recombinant host cell, or synthetic production. Recombinant production can be carried out in host cells of microbial origin. Preferred yeast host cells are selected from, but are not limited to, the genera Saccharomyces, Kluyveromyces, Hansenula, Pichia, Yarrowia and Candida. Preferred bacterial host cells include Escherichia coli, Agrobacterium species, Bacillus species and Streptomyces species. Preferred filamentous fungal host cells are selected from the group consisting of the genera Aspergillus, Trichoderma, and Fusarium. The production of the polypeptide can be, e.g., intra- or extra-cellular production and can be by, e.g., secretion into the culture medium.

In these fermentation reactions of the invention, the polypeptides (including functional fragments) can be immobilized on a solid support or free in solution. Procedures for immobilization of the yeast or preparation thereof include, but are not limited to, adsorption; covalent attachment; cross-linked enzyme aggregates; cross-linked enzyme crystals; entrapment in hydrogels; and entrapment into reverse micelles.

The progress of the reaction can be monitored by standard procedures known to one skilled in the art, which include, for example, gas chromatography or high-pressure liquid chromatography on columns containing chiral stationary phases. The vicinal diol formed can be removed from the reaction mixture at one or more stages of the reaction

The reaction can be terminated when one enantiomer of the epoxide and/or vicinal diol is found to be in excess compared to the other enantiomer of the epoxide and/or vicinal diol. Preferably, the reaction is terminated when one enantiomer of an epoxide of general formula (I) and/or vicinal diol of general formula (I) is found to be in an enantiomeric excess of at least 90%. In a more preferred embodiment of the invention, the reaction is terminated when one enantiomer of an epoxide of general formula (I) and/or vicinal of general formula (II) is found to be in an enantiomeric excess of at least 95%. The reaction can be terminated by the separation (for example centrifugation, membrane filtration and the like) of the yeast, or a preparation thereof, from the reaction mixture or by inactivation (for example by heat treatment or addition of salts and/or organic solvents) of the yeast or polypeptide, or preparation thereof. Thus, the reaction can be stop for by, for example, the separation of the catalytic agent from the reactants and products in the mixture, or by ablation or inhibition of the catalytic activity, by techniques known to one skilled in the art.

The optically active epoxides and/or vicinal diols produced by the reaction can be recovered from the reaction mixture, directly or after removal of the yeast, or preparation thereof. Preferably, the process can include continuously recovering the optically active epoxide and/or vicinal diol produced by the reaction directly from the reaction mixture. Methods of removal of the optically active epoxide and/or vicinal diol produced by the reaction include, for example, extraction with an organic solvent (such as hexane, toluene, diethyl ether, petroleum ether, dichloromethane, chloroform, ethyl acetate and the like), vacuum concentration, crystallisation, distillation, membrane separation, column chromatography and the like.

Thus, the present invention provides an efficient process with economical advantages compared to other chemical and biological methods for the production, in high enantiomeric purity, of optically active epoxides of the general formula (I) and vicinal diols of the general formula (II) in the presence of a yeast strain having enantioselective epoxide hydrolase activity or a polypeptide having such activity.

### Yeast Epoxide Hydrolase Antibodies

The invention features antibodies that bind to yeast epoxide hydrolase polypeptides or fragments (e.g., antigenic or functional fragments) of such polypeptides. The polypeptides are preferably yeast epoxide polypeptides with enantioselective activity, and in particular those with styrene-type epoxide enantioselective activity (i.e., YESH), e.g., those with SEQ ID NOs: 1, 2, 3, 4, or 5. The antibodies preferably bind specifically to yeast epoxide hydrolase polypeptides, i.e., not to epoxide hydrolase polypeptides of species other than yeast species. More preferably, they can bind specifically to yeast epoxide polypeptides with enantioselective activity, and in particular to YESH polypeptides, e.g., those with SEQ ID NOs: 1, 2, 3, 4, or 5. They can moreover bind specifically to one or more of polypeptides with SEQ ID NOs: 1, 2, 3, 4, or 5.

Antibodies can be polyclonal or monoclonal antibodies; methods for producing both types of antibody are known in the art. The antibodies can be of any class (e.g., IgM, IgG, IgA, IgD, or IgE). They are preferably IgG antibodies. Moreover, polyclonal antibodies and monoclonal antibodies can be generated in, or generated from B cells from, animals any number of vertebrate (e.g., mammalian) species, e.g., humans, non-human primates (e.g., monkeys, baboons, or chimpanzees), horses, goats, camels, sheep, pigs, bovine animals (e.g., cows, bulls, or oxen), dogs, cats, rabbits, gerbils, hamsters, guinea pigs, rats, mice, birds (such as chickens or turkeys), or fish.

Recombinant antibodies specific for YESH polypeptides, such as chimeric monoclonal antibodies composed of portions derived from different species and humanized monoclonal antibodies comprising both human and non-human portions, are also encompassed by the invention. Such chimeric and humanized monoclonal antibodies can be produced by recombinant DNA techniques known in the art, for example, using methods described in Robinson et al., International Patent Publication PCT/US86/02269; Akira et al., European Patent Application 184,187; Taniguchi, European Patent Application 171,496; Morrison et al., European Patent Application 173,494; Neuberger et al., PCT Application WO 86/01533; Cabilly et al., U.S. Pat. No. 4,816,567; Cabilly et al., European Patent Application 125,023; Better et al. (1988) Science 240, 1041-43; Liu et al. (1987) J. Immunol. 139, 3521-26; Sun et al. (1987) PNAS 84, 214-18; Nishimura et al. (1987) Canc. Res. 47, 999-1005; Wood et al. (1985) Nature 314, 446-49; Shaw et al. (1988) J. Natl. Cancer Inst. 80, 1553-59; Morrison, (1985) Science 229, 1202-07; Oi et al. (1986) BioTechniques 4, 214; Winter, U.S. Pat. No. 5,225,539; Jones et al. (1986) Nature 321, 552-25; Veroeyan et al. (1988) Science 239, 1534; and Beidler et al. (1988) J. Immunol. 141, 4053-60.

Also useful for the invention are antibody fragments and derivatives that contain at least the functional portion of the antigen-binding domain of an antibody that binds to a YESH polypeptide. Antibody fragments that contain the binding domain of the molecule can be generated by known techniques. Such fragments include, but are not limited to: F(ab)₂ fragments that can be produced by pepsin digestion of antibody molecules; Fab fragments that can be generated by reducing the disulfide bridges of F(ab′)₂ fragments; and Fab fragments that can be generated by treating antibody molecules with papain and a reducing agent. See, e.g., National Institutes of Health, 1 Current Protocols In Immunology, Coligan et al., ed. 2.8, 2.10 (Wiley Interscience, 1991). Antibody fragments also include Fv fragments, i.e., antibody products in which there are few or no constant region amino acid residues. A single chain Fv fragment (scFv) is a single polypeptide chain that includes both the heavy and light chain variable regions of the antibody from which the scFv is derived. Such fragments can be produced, for example, as described in U.S. Pat. No. 4,642,334, which is incorporated herein by reference in its entirety. The antibody can be a “humanized” version of a monoclonal antibody originally generated in a different species.

The above-described antibodies can be used for a variety of purposes including, but not limited to, YESH polypeptide purification, detection, and quantitative measurement.

The following examples serve to illustrate, not limit, the invention.

### EXAMPLES

### Example I

### Materials and Methods

### Preparation of Frozen Yeast Cells for Screening

Yeasts were grown at 30° C. in 1 L shake-flask cultures containing 200 ml yeast extract/malt extract (YM) medium (3% yeast extract, 2% malt extract, 1% peptone w/v) supplemented with 1% glucose (w/v). At late stationary phase (48-72 h) the cells were harvested by centrifugation (10 000 g, 10 min, 4° C.), washed with phosphate buffer (50 mM, pH7.5), pelleted by centrifugation, and frozen in phosphate buffer containing glycerol (20%) at −20° C. as 20% (w/v) cell suspensions. The cells were stored for several months without significant loss of activity.

### Yeast Isolate (Strain) Screening

Epoxide (10 μl of a 1 M stock solution in EtOH) was added to a final concentration of 20 mM to 500 μl cell suspension (20% w/v) in phosphate buffer (50 mM; pH 7.5). The reaction mixtures were incubated at 30° C. for 5 hours, extracted with EtOAc (300 μl), and centrifuged. Vicinal diol formation was evaluated by thin layer chromatography (TLC) (silica gel Merck 60 F₂₅₄). Compounds were visualized by spraying with vanillin/conc. H₂ SO₄ (5 g/l). Reaction mixtures that showed substantial vicinal diol formation were evaluated for asymmetric hydrolysis of the epoxide by chiral GLC analysis. Some reactions were repeated over longer or shorter times in order to analyse the reactions at suitable conversions.

### General Procedure for the Hydrolysis of (Substituted) Styrene Epoxides

Frozen yeast cells were thawed, washed with phosphate buffer (50 mM, pH 7.5) and resuspended in buffer. Cell suspensions (10 ml, 20% or 50% w/v) were placed in 20 ml glass bottles with screw caps fitted with septa. The substrate (100 or 250 μl of a 2M (v/v) stock solution in ethanol) was added to final concentrations of 20 mM or 50 mM. The mixtures were agitated on a shaking water bath at 30° C. The course of the bioconversions of epoxides was followed by withdrawing samples (500 μl) at appropriate time intervals. Samples were extracted with 300 μl EtOAc. After centrifugation (3000×g, 2 min), the organic layer was dried over anhydrous MgSO₄, and the products analyzed by chiral GLC.

### Determination of the Absolute Configuration of Residual Styrene Epoxide and Phenyl Ethanediol

Absolute configurations were deduced from reported elution orders of the epoxide and diol enantiomers on cyclodextrin columns and verified by co-injection of the commercially available (R)-3-chlorostyrene oxide (Aldrich).

### Determination of Concentrations and Enantiomeric Excesses

In the various working examples below, quantitative determinations of the compounds and determination of enantiomeric excesses were carried out by GC. Gas chromatography (GLC) was performed on a Hewlett-Packard 6890 gas chromatograph equipped with a FID detector and using H₂ as carrier gas. Chiral analysis of styrene epoxides and phenylethanediol was done on fused capillary cyclodextrin columns (30 m length, 25 mm ID and 25 μm film thickness) from Supelco (head pressure of 10-14 psi) at isotherms given below:

| | | Column | |
|---|---|---|---|
| | Chiral | temper- | |
| Compound | column | ature | Retention time (min) |
| Styrene oxide: | β-Dex 225 | 90° C. | (S)-: 8.00; (R)-: 8.23 |
| 2-Chlorostyrene oxide | α-Dex 110 | 90° C. | (S)- 2.69; (R)-: 23.12 |
| 3-Chlorostyrene oxide | β-Dex 225 | 100° C. | (S)-: 7.20; (R)-: 7.40 |
| 4-Chlorostyrene oxide | β-Dex 225 | 100° C. | (S)- 9.35 (R)-: 9.50 |
| 2-Nitrostyrene oxide | β-Dex 225 | 130° C. | (S)-: 12.12; (R)-: 12.36 |
| 3-Nitrostyrene oxide | β-Dex 225 | 150° C. | (S)-: 11.98; (R)-: 12.25 |
| 4-Nitrostyrene oxide | β-Dex 225 | 150° C. | (S)-: 18.16; (R)-: 18.99 |
| Phenylethanediol | β-Dex 120 | 150° C. | (S)-: 11.76; (R)-: 12.25 |

### Synthesis of Chloro-Substituted Styrene Epoxides

### (a) Synthesis of 2-Chlorostyrene Epoxide

A solution of 2-chlorostyrene (10.820 g, 78.066 mmol) in chloroform (200 cm³, 0.4 M) was treated with 10 g dried magnesium sulphate and cooled to 0° C. meta-Chloroperoxybenzoic acid (23.094 g, 93.680 mmol, 1.2 eq.) was then added, with effervescence, and the resultant yellow suspension left to stir for 1 h at 0° C. The mixture was decanted into 250 cm³ saturated aqueous sodium bicarbonate solution and extracted with chloroform (3×100 em). Drying and concentration afforded a yellow oil, which was purified by column chromatography [1:10 (v/v) ethyl acetate:hexane as eluent] to afford a pale yellow oil, 2-chlorostyrene epoxide (8.693 g, 72%). δ_{H} (200 MHz, CDCl₃) 7.34-7.41 (1H, m, H-3), 7.27-7.30 (3H, m, H-4, H-5 and H-6), 4.23 (1H, dd, ArCH—, J 4.0 and 2.4), 3.21 (1H, dd, CHCHₐH_{b}, J 6.0 and 4.2) and 2.69 (1H, dd, CHCHₐH_{b}, J 5.8 and 2.4).

### (b) Synthesis of 3-Chlorostyrene Epoxide

A solution of 3-chlorostyrene (52.72 g, 0.380 mol) in dichloromethane (250 cm³, 1.5 M) was treated with 50 g dried magnesium sulphate and cooled to 0° C. meta-Chloroperoxybenzoic acid (140.66 g, 0.571 mol, 1.5 eq.) was then added, with effervescence, and the resultant yellow suspension left to stir for 3 h at 0° C. The mixture was decanted into 250 cm³ saturated aqueous sodium carbonate solution and extracted with dichloromethane (3×100 cm³). Drying and concentration afforded a yellow oil, which was purified by column chromatography [1:10 (v/v) ethyl acetate:hexane as eluent] to afford a pale yellow oil, 3-chlorostyrene epoxide (38.24 g, 65%). δ_{H} (200 MHz, CDCl₃) 7.15-7.36 (4H, m, aryl H), 3.86 (1H, dd, ArCH, J 4.0 and 2.4), 3.16 [1H, dd, —CHₐH_{b}, J 5.6 and 4.0] and 2.78 [1H, dd, —CHaHb, J5.4 and 2.4].

### (c) Synthesis of 4-Chlorostyrene Epoxide

A solution of 4-chlorostyrene (10.876 g, 78.470 mmol) in chloroform (200 cm³, 0.4 M) was treated with 10 g dried magnesium sulphate and cooled to 0° C. meta-Chloroperoxybenzoic acid (22.229 g, 94.225 mmol, 1.2 eq.) was then added, with effervescence, and the resultant yellow suspension left to stir for 1 h at 0° C. The mixture was decanted into 250 cm³ saturated aqueous sodium bicarbonate solution and extracted with chloroform (3×100 cm³). Drying and concentration afforded a yellow oil, which was purified by column chromatography [1:10 (v/v) ethyl acetate:hexane as eluent] to afford a pale yellow oil, 4-chlorostyrene epoxide (7.632 g, 63%). δ_{H} (200 MHz, CDCl₃) 7.31-7.37 (2H, m, H-3 and H-5), 7.19-7.26 (2H, m, H-2 and H-6), 3.85 (1H, dd, ArCH—, J 4.2 and 2.6), 3.16 (1-, dd, CHCHₐH_{b}, J 5.4 and 4.2) and 2.77 (1H, dd, CHCHₐH_{b}, J 5.4 and 2.4).

### Synthesis of Nitro-Substituted Styrene Epoxides

2-nitrostyrene epoxide and 4-nitrostyrene epoxide was synthesized from 2-bromo-2′ nitroacetophenone and 2-bromo-4′ nitroacetophenone as previously reported (Pedragosa-Moreau et al., 1996). 3-nitrostyrene epoxide was synthesized from 3-nitroacetophenone as described below.

### (a) Synthesis of 3-Nitroacetophenone

A mixture of acetophenone (30.018 g, 0.250 mol) in concentrated sulphuric acid (75 cm³, 6.9 M) was cooled to 0° C. in an ice-salt bath. An ice cold mixture of 65% nitric acid (20 cm³, 0.206 mol, 0.83 eq.) in concentrated sulfuric acid (30 cm³, 6.9 M) was then added dropwise with vigorous stirring over 45 minutes while maintaining the internal temperature below 0° C., affording a dense orange-yellow gum. This was poured onto 300 cm³ of crushed ice in 750 cm³ water, stirred for 30 minutes and the resultant yellow powder, 3-nitroacetophenone (19.293 g, 57%) was isolated by filtration. δ_{H} (200 MHz, CDCl₃) 8.74 (1H, d, H-2, J 2.0), 8.35 (1H, dd, H-6, J 8.2 and 2.4), 8.29 (1H, dd, H-4, J 7.8 and 2.2), 7.69 (1H, dd, H-5, J 8.2 and 7.8) and 2.69 (3H, s, CH₃CO).

### (b) Synthesis of 3-Nitrostyrene Epoxide

3-Nitroacetophenone (5.048 g, 30.567 mmol) in anhydrous tetrahydrofuran (230 cm³, 0.1 M) was cooled to 0° C. Aluminium chloride (0.155 g, 3 g.mol⁻¹) was then added to the solution, followed by dropwise addition of bromine (2.0 cm³, 39.4 mmol, 1.3 eq.) in anhydrous tetrahydrofuran (38 cm³, 1 M) over 1 h to afford a yellow solution. The mixture was warmed over 1 h to room temperature, then concentrated to a viscous orange oil. This was crystallised from ethyl acetate:hexane to afford a beige solid, the bromoacetophenone, which was approximately 80% monobromide to 20% starting material. Monobromide: δ_{H} (200 MHz, CDCl₃) 8.49 (1H, d, H-2, J 2.0), 8.10 (2H, dd, H-4 and H-6, J 7.6 and 2.2), 7.69 (1H, d, H-5, J 8.2) and 4.42 (3H, s, CH₃ CO). The material was dissolved in methanol (46 cm³, 0.4 M) and cooled to 0° C. Sodium borohydride (0.905 g, 23.924 mmol, 1.3 eq.) was then added, with much effervescence, and left for 20 minutes whereupon TLC analysis revealed complete reduction had occurred. A chilled solution of 2 M aqueous sodium hydroxide (65 cm³, 0.13 mol, 7 eq.) was then added, and the orange mixture left to stir for 2 h at 0° C. Extraction with dichloromethane (3×50 cm³), drying, and concentration afforded an orange oil. Column chromatography using 1:10 (v/v) ethyl acetate:hexane as eluent afforded a yellow oil, 3-nitrostyrene epoxide (1.377 g, 44% over two steps). δ_{H} (200 MHz, CDCl₃) 8.15-8.21 (2H, m, H-2 and H-6), 7.53-7.68 (2H, m, H-4 and H-5), 3.99 (1H, dd, ArCH—, J 4.0 and 2.4), 3.23 (1H, dd, CHCHₐH_{b}, J 5.4 and 4.0) and 2.82 (1H, dd, CHCHₐ H_{b}, J5.4 and 2.6).

### Yeast Strains

Yeast strains with the “Jen” designation and numerical screen numbers were obtained from the Yeast Culture Collection of the University of the Free State. Yeast strains with “AB” or “Car” or “Alf” or “Poh” designations were isolated from soil from specialised ecological niches. “AB” and “Alf” strains were isolated from Cape Mountain fynbos, an ecological environment unique to South Africa, “Car” strains were isolated from soil under pine trees, and “Poh” strains from soil contaminated by high concentrations of cyanide. It seemed likely that microorganisms existing in these contaminated soils would have alternative respiratory mechanisms.

All new isolates that produced optically active epoxides or vicinal diols during hydrolysis of SEQ were identified by known biochemical characterisation methods, and most of the isolates were also subjected to molecular identification by sequence analyses of the D1/D2 region of the large subunit rDNA. These new isolates were subsequently deposited at the Yeast Culture Collection of the University of the Orange Free State (UOFS) and assigned UOFS numbers. Some of the isolates were deposited at the National Collection of Yeast Cultures (NCYC) under the Budapest Treaty.

Cultivation of Rhodosporidium toruloides (NCYC 3181) in a 10 L Volume in a 15 L Fed-Batch Bioreactor

Cultivation was performed at 25° C. in 15 L Braun Biostat C bioreactors (working volume 10 L). An overpressure of 500 mbar was applied to the reactor. Dissolved oxygen was continuously monitored and maintained at 30% saturation by adjustment of the stirrer speed. Airflow rate was controlled at 5.5 L.min⁻¹ by use of a mass flow meter. pH was automatically maintained at 5.5±0.05 by the addition of 25% (w/v) NH₄OH. Excessive foam formation was avoided by the addition of antifoam (Pluriol P 2000).

A Fernbach shake flask inoculum (10%, v/v) of Rhodosporidiun toruloides (NCYC 3181) was transferred into a 15 L Braun Biostat C bioreactor containing 6 L medium with the following composition (per litre): citric acid, 2.5 g; yeast extract, 7 g; (NH₄)₂SO₄, 58 g; KH₂ PO₄, 11.3 g; MgSO₄.7H₂O, 8.2 g; CaCl₂.2H₂O, 0.88 g; NaCl, 0.1 g; H₃PO4, 13.4; vitamin solution, 1.7 ml; trace element solution, 1 ml; antifoam (Pluriol P-2000), 0.5 ml; and glucose, 20 g. Glucose (60% m/m) was fed to maintain a residual glucose concentration of 5 g/l after the batch phase. Glucose feed was stopped when the glucose uptake rate decreased. Cultivation was continued for 12 hours after the residual glucose concentration was zero. The biomass was harvested by centrifugation and frozen at −20° C. in phosphate buffer pH 7.5 containing 20% glycerol until use.

### Example II

### Sequencing, Cloning and Overexpression of Wild Type Yeast Epoxide Hydrolases in Yarrowia lipolytica as Production Host under the Control of Different Promoters

### 1. Vectors, Strains and Primers (Table 1)

The following features are common to all the E. coli_{—}/Y. lipolytica auto-cloning integrative vectors used:
LIP2 terminator
Zeta regions
Kanamycin resistance for E. coli selection
mono-copy auto cloning vectors (pINA 1311, pINA 1313, pINA 3313) with a fully functional selection marker gene carry the fully functional ura3d1 allele from the URA3 selection marker gene
multi-copy auto cloning vectors (pINA 1291, pINA 1293, pINA 3293) with a defective selection marker gene (copy number amplification) carry the defective ura3d4 allele from the URA3 selection marker gene.

TABLE 1
Vectors, strains and primers

| | |
|---|---|
| Description | Cloning sites |

| | | Selection | Targeting | Upstream/down | Reference/ |
|---|---|---|---|---|---|
| Vectors | Promoter | marker | sequence | stream | Origin |
| pINA 1291 | hp4d | ura3d4 | none | PmlI (blunt)/ | Nicaud et al |
| (pYLHmA) | | | | BamHI, KpnI, AvrII | (2002) |
| pYL3313 | TEF | ura3dI | none | XmnI (in pro)/ | This study |
| (1313) | | | | BamHI, KpnI, AvrII | |
| (pYLTsA) | | | | | |

| | | Reference |
|---|---|---|
| Host Strain | Description | Origin |
| Yarrowia | MATA, ura3-302, uxpr2-322, axpl-2 (deleted for both | GLIB 882 |
| lipolytica Polh | extracellular proteases and growth on sucrose) | |

| Primers | Sequence | Specifications |
|---|---|---|
| YL-fwd | 5′-GGA GTT CTT CGC CCA C-3′ | amplification of expression |
| | (SEQ ID NO: 11) | casette between NotI sites |
| YL-rev | 5′-GAT CCC CAC CGG AAT TG-3′ | |
| | (SEQ ID NO: 12) | |
| pINA-1 | 5′-CAT ACA ACC ACA CAC ATC CA-3′ | PYLHmA fwd primer |
| | (SEQ ID NO: 13) | |
| pINA-2 | 5′-TAA ATA GCT TAG ATA CCA CAG-3′ | pYLTsA/pYLHmA rev primer |
| | (SEQ ID NO: 14) | |
| pINA-3 | 5′-CTC TCT CTC CTT GTC AAC T-3′ | pYLTsA fwd primer |
| | (SEQ ID NO: 15) | |

| Transformants | Gene origin |
|---|---|
| TEF promoter | Vector: pYL3313 (1313) = (pYLTsA) |
| YL23TsA | Rhodotorula mucilaginosa NCYC 3190 |
| YL25TsA | Rhodotorula araucariae NCYC 3183 |
| YL46TsA | Rhodosporidium toruloides UOFS Y-0471 |
| YL692TSA | Rhodosporidium paludigenum NCYC 3179 |
| YL1TsA | Rhodosporidium toruloides NCYC 3181 |
| YLCar54 TsA | Cryptococcus curvatus NCYC 3158 |
| hp4d promoter | Vector: pINSA1291 = (pYLHmA) |
| YL25HmA | Rhodotorula araucariae NCYC 3183 |
| YL46HmA | Rhodosporidium toruloides UOFS Y-0471 |
| YL692HmA | Rhodosporidium paludigenum NCYC 3179 |

### 3. Vector Preparation

pINA1291 (FIG. 1) was obtained from Dr Madzak of Labo de Génétique, INRA, CNRS. This vector was renamed pYLHmA (Yarrowia Lipolytica expression vector, with Hp4d promoter, multi-copy integration selection, Absent secretion signal; e.g. for construction of recombinant strain YL-25HmA).

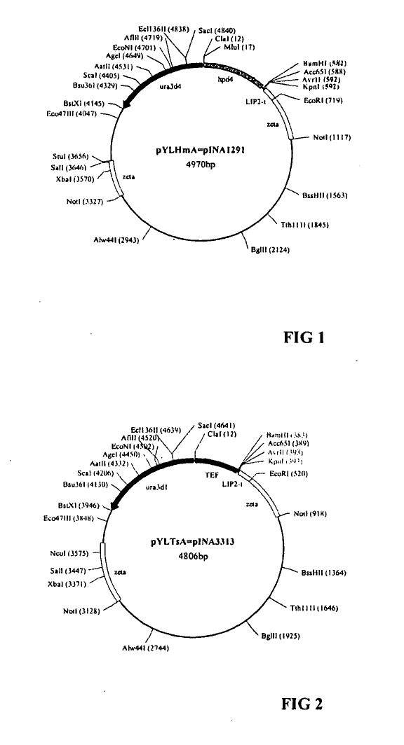

pINA3313 (pKOV93) (FIG. 2) was prepared by the inventors. This vector was renamed pYLTsA (Yarrowia Lipolytica expression vector, with TEF promoter, single-copy integration selection, Absent secretion signal; e.g. for construction of recombinant strain YL-25TsA).

To prepare the vectors for ligation with an epoxide hydrolase coding sequence (or other insert to be expressed in Y. lipolytica), DNA was digested with BamHI and AvrII.

### 4. Insert Preparation

Total RNA was isolated from selected yeast strain cells and messenger RNA (mRNA) was purified from it The mRNA was used as a template to synthesise complementary DNA (cDNA) using reverse transcriptase. The cDNA was then used as a template for Polymerase Chain Reaction (PCR) using appropriate primers. PCR primers were selected by repeated experimentation using multiple test primers for each yeast strain, the sequences of which were based on previously described epoxide hydrolase sequences from a variety of species. The nucleotide sequences of the forward and reverse primers used to generate cDNA coding sequences from mRNA from seven different yeast strains with appropriate restriction enzyme recognition sites at their termini are shown below. Restriction enzyme recognition sequences are underlined and the relevant restriction enzymes are shown in parentheses.

| | | |
|---|---|---|
| Strain | 5′ primer | 3′ primer |

| | | |
|---|---|---|
| R. toruloides | GTGGATCCATGGCGACACACA | GACCTAGGCTACTTCTCC |
| NCYC 3181 (#1) | (BamHI) | CACA (BlnI) |
| R.toruloides | (SEQ ID NO: 16) | (SEQ ID NO: 17) |
| UOFS Y-0471 (#46) | | |
| C. curvatus | | |
| NCYC 3158 (Car 054) | | |
| R. araucariae | GATTAATGATCAATGAGCGAG | GACCTAGGTCACGACGAC |
| NCYC 3183 (#25) | CA (BclI) (SEQ ID NO: 18) | AG (BlnI) (SEQ ID NO: 19) |
| R. paludigenum NCYC | GTGGATCCATGGCTGCCCA | GAGCTAGCTCAGGCCTGG |
| 3179 (#692) | (BamHI) (SEQ ID NO: 20) | (NheI) (SEQ ID NO: 21) |
| R. mucilaginosa NCYC | GTATATCTATGCCCGCCCGCT | GACCTAGGCTACGATT |
| 3190 (#23) | (BglII) (SEQ ID NO: 22) | TTTGCT (BlnI) |
| | | (SEQ ID NO: 23) |
| Y.lipolytica | GCAGATCTATGTCATCACTCG | GACCTAGGCTACAACTTC |
| NCYC 3229 (Jen 46-2) | (BglII) (SEQ ID NO: 24) | GACG (BlnI) |
| | | (SEQ ID NO: 25) |

Each PCR reaction contained 200 μM dNTPs, 250 nM of each primer, 2 mM of MgCl₂, cDNA and 2.5 U of Taq polymerase in a 50 μl reaction volume. The PCR profile used was: 95° C. for 5 minutes, followed by 30 cycles of: 95° C.-1 min, 50° C.-1 main, 72° C.-2 min, then a final extension of 72° C. for 10 minutes. The PCR products were purified and digested with the restriction enzymes whose recognition sites are engineered at the end of the primers. The cDNA fragment was cloned into a vector and sequenced for confirmation.

Coding sequences to be inserted in either pYLHmA or pYLTsA were prepared with BamHI and AvrII at their termini. The above PCR primers were designed with these restriction sites, unless the sites were also present in the gene to be inserted. If this occurred, appropriate compatible restriction enzymes were selected. PCR template DNA was either the insert cloned into a different vector, or cDNA synthesized from the original host organism. PCR reactions consisted of 200 μM dNTP's, 250 nM each primer, 1× Taq polymerase buffer, and 2.5 units Taq polymerase per 100 μl reaction. The amplification programme used was: 95° C. for 5 minutes, 30 cycles of 95° C. for 1 minute, 50° C. for 1 minute, and 72° C. for 2 minutes, followed by a single duration at 72° C. for 10 minutes.

PCR products were purified and digested with the relevant restriction enzymes. The digested DNA was subsequently repurified and was ready for ligation into the prepared vector.

5. Preparation of pYLHmA or pYLTsA Constructs

Vector and insert were ligated at pmol end ratios of 3:1-10:1 (insert:vector), using commercial T4 DNA Ligase. Ligations were electroporated into any laboratory strain of Escherchia coli, using the Bio-Rad GenePulser, or equivalent electroporator. Transformants were selected on LM media (10 g/L yeast extract, 10 g/L tryptone, 5 g/L NaCl), supplemented with kanamycin (50 μg/nl). Transformants were selected based on restriction enzyme digests of purified plasmid DNA.

6. Yarrowia lipolytica Transformation

### 6.1.1. Preparation of DNA—Method 1

Digestion of the pINA-series of plasmids with NotI resulted in the release of a bacterial DNA-free expression cassette, containing the ura3d4 (pYLHmA) or the ura3d1 (pYLTsA) marker gene and the promoter-gene-terminator.

Scaled-up quantities of each plasmid were isolated. NotI was used to restrict the plasmid DNA, and the digested DNA was run on an agarose gel. Nod digests resulted in generation of the bacterial fragment of the plasmid as a band at 2210 bp, and the expression cassette as a band of 2760 bp+size of insert (pYLHmA) or 2596 bp+size of insert (pYLTsA). The expression cassette fragments were excised from the gel and purified from the agarose. The purified fragment was used for transformation of Y. lipolytica Po1h.

### 6.1.2. Preparation of DNA—Method 2

Primers YL-Fwd and YL-Rev (Table 1) were used to amplify the expression cassette. PCR reactions consisted of 200 μM dNTP's, 250 pmol each primer, 1× Taq polymerase buffer and 2.5 units Taq polymerase per 100 μl reaction. The amplification programme used was 95° C. for 5 minutes, 30 cycles of 95° C. for 1 minute, 50° C. for 1 minute, and 72° C. for 3½ minutes, followed by a single duration at 72° C. for 10 minutes. The PCR product was purified from the PCR reaction mix and used for transformation of Y. lipolytica Po1h.

### 6.13. Preparation of Carrier DNA

DNA from salmon testes was made up as a 10 mg/ml stock in TE (10 mM Tris-HCl, pH 8.0, 1 mM EDTA) and sonicated to result in fragments that ranged from approximately 15 kb to 100 bp, with most fragments in a range of 6 to 10 kb. The DNA was denatured by boiling. Aliquots were stored at −20° C.

6.1.4. Transformation of Yarrowia lipolytica with pYLHmA or pYLTsA

An adaptation of the method of Xuan et al (1988) was used for the transformation of Y. lipolytica Po1h. The yeast was inoculated into 50 ml YPD (10 g/L yeast extract, 20 g/L peptone, 20 g/L glucose). The culture was incubated at 30° C., 220 rpm until cell densities of 8×10⁷-2×10⁸ cells/ml were reached. The entire culture was harvested, the pellet resuspended in 10 ml TE and reharvested. 1 ml TE+0.1 M LiOAc was used to resuspend the pellet and the culture was incubated at 28° C. in a ProBlot Jr (Labnet) hybridisation oven, set at 4 rpm (or similar incubator) for 1 hour. Transformation mixes were set up with 0.5-2 μg of transforming DNA+5 μg of carrier DNA with 100 μl of treated cells.

Each mix was set up in a 1.5 ml microfuge tube, and incubated in a 28° C. heating block for 30 minutes. 7 volumes of PEG reagent (40% PEG 4,000, 0.1 M LiOAc, 10 mM Tris, 1 mM EDTA, pH 7.5, filter-sterilised) were added to each, mixed carefully and incubated at 28° C. for a further 1 hour. The tubes were transferred to a 37° C. heating block for 15 minutes, and then the cells were pelleted by centrifugation for 1 minute at 13,000 rpm. The cell pellets were carefully resuspended in 100 μI dH₂O. The transformations were plated on Y. lipolytica selective plates (17 g/l Difco yeast nitrogen base without amino acids and without (NH₄)₂SO₄, 20 g/L glucose, 4 g/L NH₄Cl, 2 g/L casamino acids, 300 mg/L leucine) and incubated at 28° C.

Colonies which appeared on the selective plates after 3-7 days were transferred onto fresh plates and regrown.

6.1.5. Confirmation of Integration of pYLHmA or pYLTsA

Colonies that grew on the newly-streaked selective plates were inoculated into 5 ml of YPD medium and grown at 30° C., 200 rpm for 24-48 hours. A small-scale genomic DNA isolation was performed.

PCR was performed using this genomic DNA as template, with either pINA-1 and pINA-2 as primers (transformants with pYLHmA), or pINA-3 and pINA-2 (pYLTsA) (Table 1). Each PCR reaction contained 200 μM dNTPs, 250 nM of each primer, 2 mM of MgCl₂, genomic DNA and 2.5 U/50 μl of Taq polymerase. The PCR profile was as described above in this example. These primer sets gave products the size of the inserted genes.

### Example III

### Selection of Wild Type Yeasts that are able to Produce Optically Active Styrene Epoxide and Phenyl Ethanediol from Unsubstituted (±)-Styrene Epoxide

Yeasts were cultivated, harvested and frozen as described above. Racemic unsubstituted SEQ was added and the screening was performed as described above. Strains with the highest activities as judged by TLC from diol formation were subjected (as Samples 1-23) to chiral GC analysis as described above (Table 2). Enantiomeric excesses (ee) were determined from the formula: ee=[[S]−[R]}/{[S]+[R]}. [S] is the concentration at the appropriate time point of the reaction of (S)-SEO enantiomer and [R] is the concentration at the appropriate time point of the reaction of the (R)-SEO enantiomer. A positive ee value indicates the relevant yeast (or yeast-derived epoxide hydrolase) is enantioselective for the R-enantiomer of the SEO and negative ee value indicates the relevant yeast (or yeast-derived epoxide hydrolase) is enantioselective for the S enantiomer of the SEO.

TABLE 2
Conversion or racemic unsubstituted styrene oxide and enantiomeric excess of
the residual (S)-styrene oxide in the reaction after exposure to selected wild type yeast
strains. Reaction conditions: 20 mM (R/S) styrene oxide, 20% cells (wet w/v), 25° C.

| | Internal | | | | (S)- | (R)- | | |
|---|---|---|---|---|---|---|---|---|
| Sample | strain | | Culture | Time | Styrene | Styrene | Conv | ee |
| No. | no. | Species | collection no. | (min) | oxide | oxide | (%) | (%) |
| 1 | #751 | Candida magnoliae | UOFS Y-1040 | 120 | 8.26 | 6.05 | 28.46 | 15.40 |
| 2 | #678 | Candida magnoliae | UOFS Y-1297 | 180 | 9.86 | 8.04 | 10.50 | 10.15 |
| 3 | Car-54 | Cryptococcus curvatus | NCYC 3158 | 120 | 7.04 | 1.65 | 56.54 | 62.07 |
| 4 | AB-29 | Cryptococcus podzolicus | UOFS Y-1897 | 120 | 9.32 | 6.12 | 22.81 | 20.75 |
| 5 | AB-37 | Cryptococcus podzolicus | UOFS Y-1896 | 180 | 8.26 | 3.68 | 40.32 | 38.41 |
| 6 | AB-39 | Cryptococcus podzolicus | UOFS Y-1912 | 120 | 9.16 | 5.35 | 27.47 | 26.26 |
| 7 | AB-50 | Cryptococcus podzolicus | UOFS Y-1883 | 180 | 8.67 | 4.01 | 36.60 | 36.73 |
| 8 | AB-52 | Cryptococcus podzolicus | UOFS Y-1895 | 120 | 9.47 | 5.72 | 24.05 | 24.64 |
| 9 | AB-56 | Cryptococcus podzolicus | UOFS Y-1913 | 120 | 9.38 | 5.02 | 27.98 | 30.26 |
| 10 | #692 | Rhodosporidium paludigenum | NCYC 3179 | 120 | 9.11 | 4.85 | 30.22 | 30.53 |
| 11 | POH-20 | Rhodosporidium toruloides | NCYC 3216 | 120 | 9.00 | 3.72 | 63.61 | 42.46 |
| 12 | POH-33 | Rhodosporidium toruloides | NCYC 3218 | 120 | 8.85 | 1.85 | 53.49 | 65.42 |
| 13 | POH-38 | Rhodosporidium toruloides | NCYC 3219 | 120 | 9.61 | 4.71 | 71.60 | 34.18 |
| 14 | AB 1 | Rhodosporidium toruloides | NCYC 3181 | 120 | 6.56 | 0.98 | 62.26 | 73.95 |
| 15 | AB 2 | Rhodosporidium toruloides | UOFS Y-0518 | 120 | 7.08 | 1.22 | 58.48 | 70.57 |
| 16 | Car-52 | Rhodosporidium toruloides | UOFS Y-2230 | 120 | 7.31 | 1.59 | 55.47 | 64.25 |
| 17 | Car-200 | Rhodosporidium toruloides | UOFS Y-2256 | 180 | 7.85 | 3.45 | 43.51 | 38.95 |
| 18 | EP-230 | Rhodotorula auranliacu | NCYC 3185 | 180 | 8.55 | 5.91 | 27.66 | 18.24 |
| 19 | #50 | Rhodotorula glutinis | NCYC 3186 | 120 | 8.79 | 6.53 | 23.42 | 14.78 |
| 20 | #680 | Rhodotorula glutinis | UOFS Y-0459 | 30 | 8.74 | 3.21 | 40.26 | 46.32 |
| 21 | #681 | Rhodotorula glutinis | UOFS Y-0653 | 60 | 9.10 | 2.85 | 40.25 | 52.23 |
| 22 | #713 | Rhodotorula minuta | NCYC 3187 | 120 | 8.84 | 4.10 | 35.26 | 36.62 |
| 23 | #232 | Trichosporon cutaneum var. | NCYC 3202 | 240 | 8.51 | 3.42 | 40.32 | 42.65 |
| | | cutaneum | | | | | | |

All the yeast strains referred to in this and the following examples are kept and maintained at the University of the Orange Free State (UOFS), Department of Microbial, Biochemical and Food Biotechnology, Faculty of Natural and Agricultural Sciences, P.O. Box 339, Bloemfontein 9300, South Africa (Tel +27 51 401 2396, Fax+27 51 444 3219) and are readily identified by the yeast species and culture collection number as indicated. Representative examples of strains belonging to the different species have been deposited under the Budapest Treaty at National Collection of Yeast Cultures (NCYC), Institute of Food Research Norwich Research Park Colney, Norwich NR4 7UA, U.K. (Tel: +44-(0)1603-255274 Fax: +44-(0)1603-458-414 Email: ncyc@bbsrc.ac.uk) and are readily identified by the yeast species and culture collection accession number as indicated. The samples deposited with the NCYC are taken from the same deposit maintained by the South African Council for Scientific and Industrial Research (CSIR) since prior to the filing date of this application. The deposits will be maintained without restriction in the NCYC depository for a period of 30 years, or 5 years after the most recent request, or for the effective life of the patent, whichever is longer, and will be replaced if the deposit becomes non-viable during that period. Samples of the yeast strains not deposited at NCYC will be made available upon request on the same basis and conditions of the Budapest Treaty.

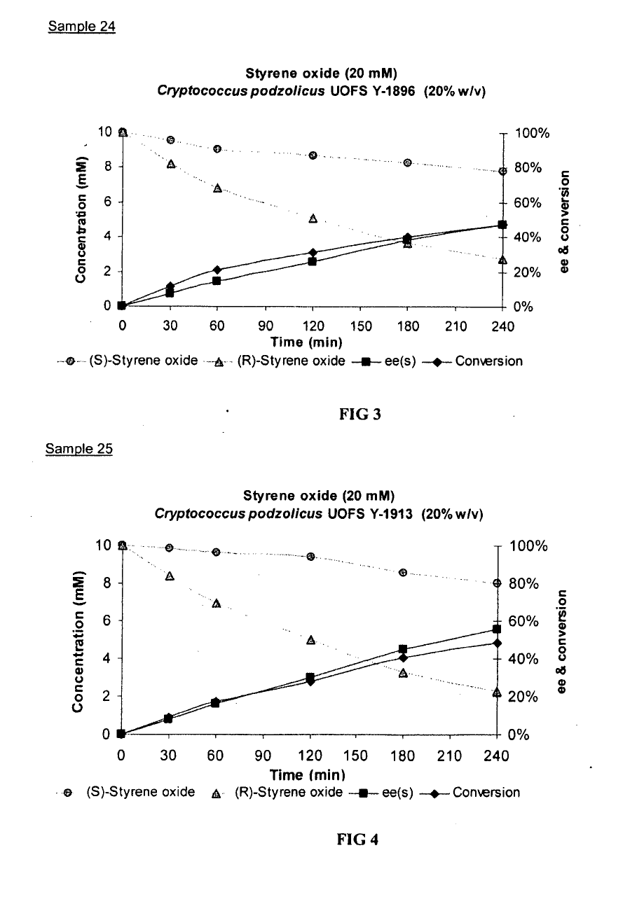

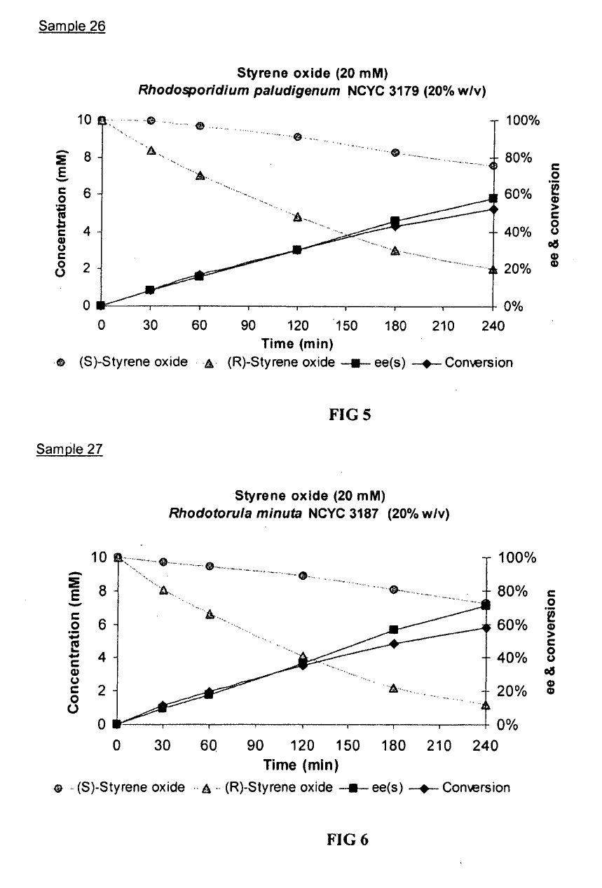

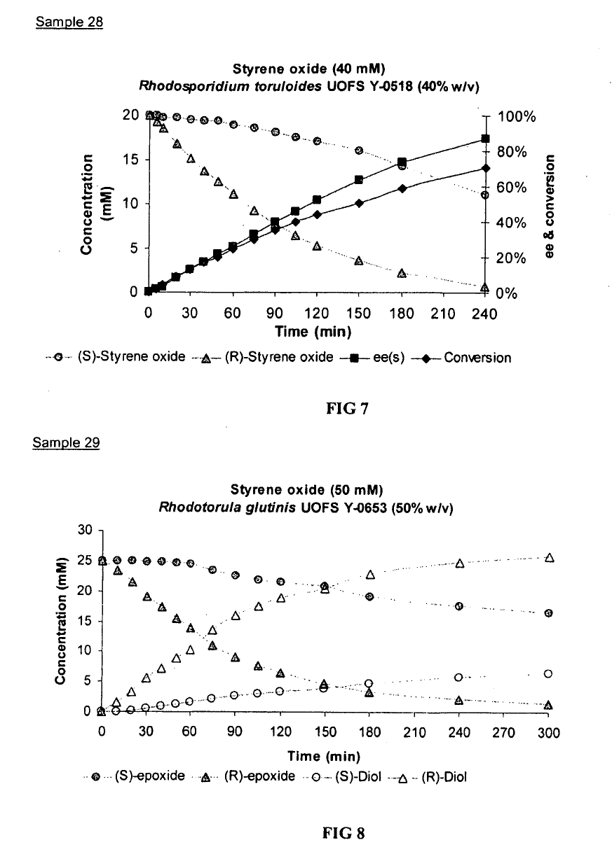

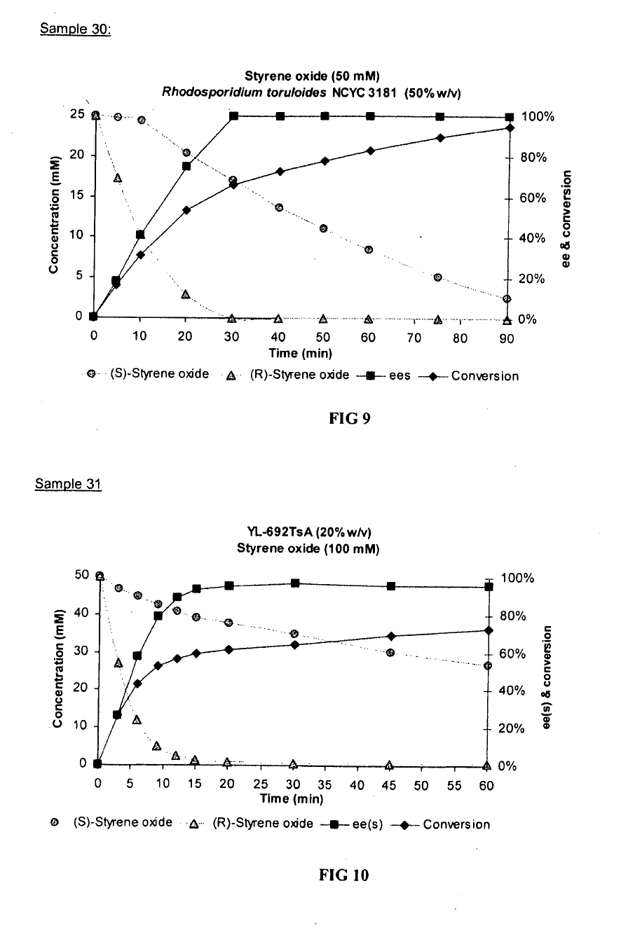

Various wild-type yeast strains selected from Table 2 were used (as Samples 24-30) to produce optically active unsubstituted SEQ and optically active unsubstituted phenylethanediol from racemic unsubstituted SEQ. For each example, a line graph is supplied that shows the change in concentrations of the epoxide and diol enantiomers with time (left y-axis) and the enantiomeric excess (ee) of the epoxide and conversion to diol at different times where the degree of conversion means the molar amount of epoxide converted to diol as a percentage (%) of the starting total molar amount of both epoxide enantiomers (right y-axis) (FIGS. 3-9). The yield of the optically active epoxide that can be obtained at a particular enantiomeric purity can be seen from these graphs where the yield (%)=100%−conversion (%).

### Example IV

### Production of Optically Active (S)-Styrene Epoxide and (R)-Phenylethanediol using Host Yeast Cells Transformed with the Epoxide Hydrolase Genes from Selected Wild Type Yeast Strains

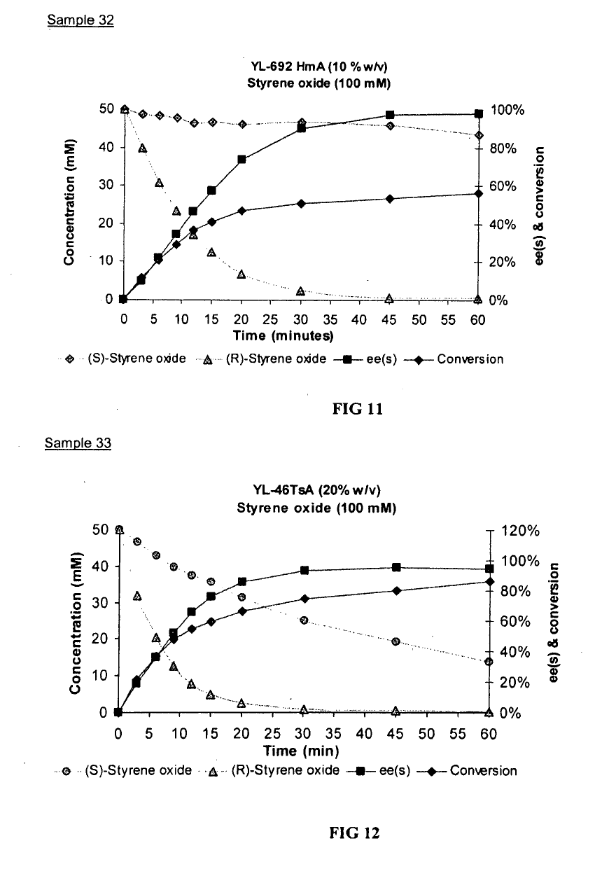

FIGS. 10-12 show the hydrolysis of (±) racemic unsubstituted SEQ by recombinant yeast strains (tested in this example as Samples 31-33) expressing, under control of different promoters, exogenous epoxide hydrolases from selected wild-type yeast strains to produce (S)-unsubstituted SEQ and (R)-unsubstituted PED.

### Example V

### Production of Optically Active (S)-Unsubstituted-Styrene Epoxide and (R)-Unsubstituted Phenylethanediol using Whole or Lysed Yeast Host Cells Transformed with the Epoxide Hydrolase Genes from a Rhodotorula araucariae Strain

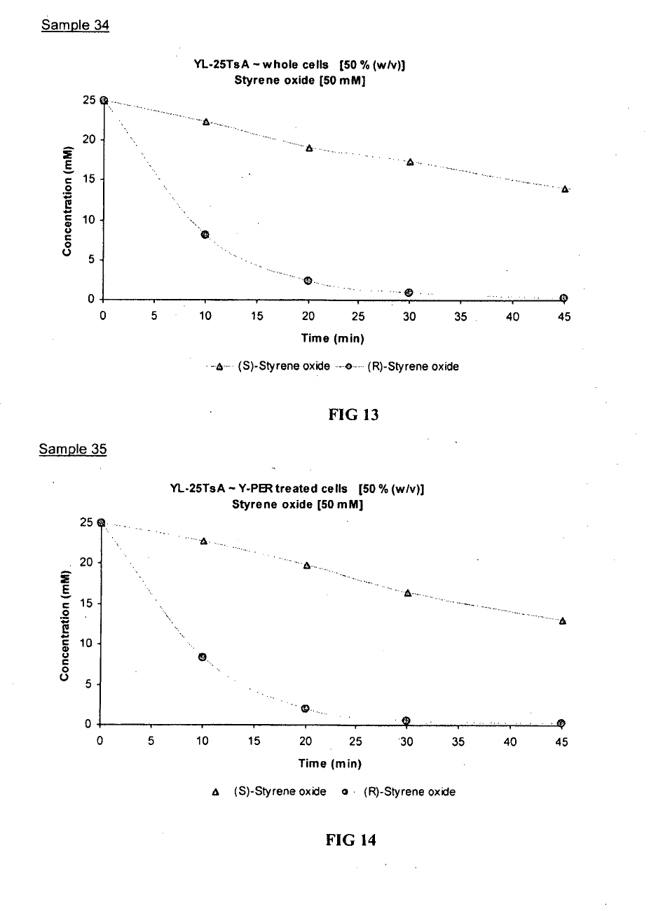

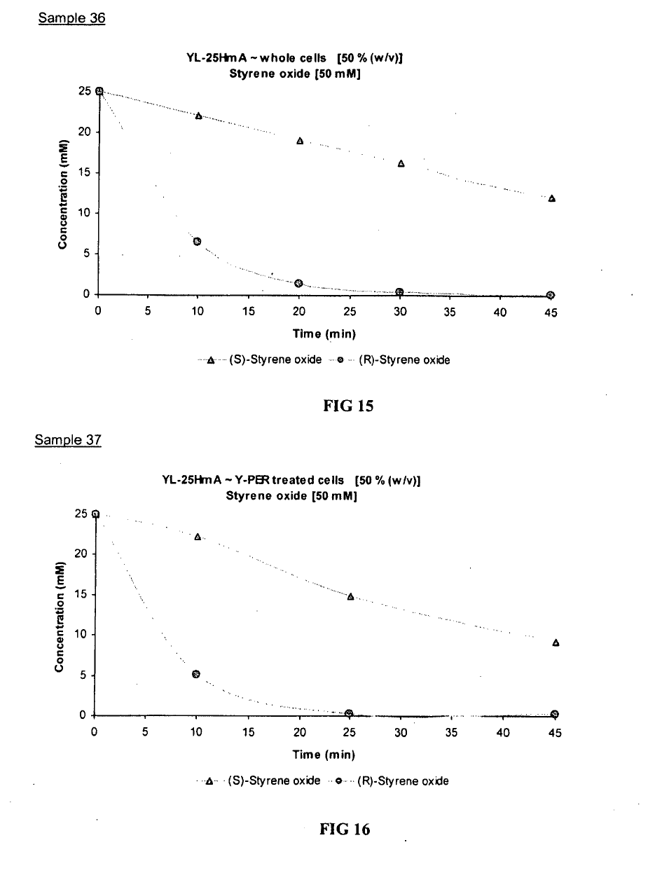

Whole cells and lysed cell suspensions of recombinant expression hosts transformed with the epoxide hydrolase gene from Rhodotorula araucariae (NCYC 3183) strains under control of two different promoters were tested (as Samples 34-37) for their ability to produce optically active (S)—SEQ and (R)-PED from (±) unsubstituted SEQ (FIGS. 13-16). Lysed cell suspensions were prepared by treatment of the cells with Y-PER®, a yeast protein extraction reagent from Sigma-Aldrich (St. Louis, Mo., U.S.A) used according to the instructions of the supplier. No significant differences in activity or enantioselectivity between and whole and lysed yeast cells were observed.

### Example VI

### Hydrolysis of (t)-Unsubstituted Styrene Oxide by a Yeast Cultivated in a 15 L Fermenter to Produce Optically Active (R)-Unsubstituted Phenylethanediol and (S)-Unsubstituted Styrene Oxide

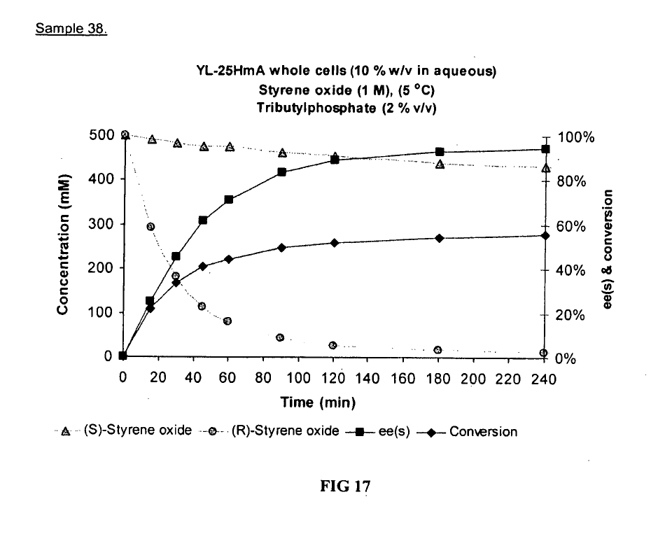

FIG. 17 shows the improvement in yields that can be achieved by process optimisation. The same recombinant strain tested in Example V was cultivated (as Sample 38 in this example) in a 10 L reaction volume in a 15 L fermenter and used to bvydrolyse (±)-unsubstituted SEQ in a stirred tank reactor at high substrate concentration (1 M SEQ in total reaction matrix) at low temperature with inclusion of 2% m/v tri-butyl phosphate (TBP) additive to improve biocatalyst stability.

### Example VII

### Phenyl-Substituted Styrene Epoxide Reactions

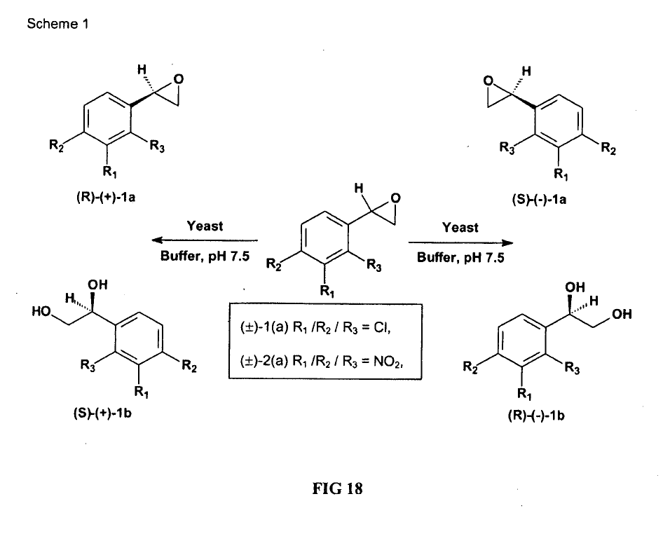

Reactions using phenyl-substituted SEQ that were used as substrates to illustrate the ability of different yeast strains with enantioselective epoxide hydrolases to hydrolyse (±)-2-, 3- or 4-substituted styrene epoxides represented by the general formula (I) are schematically depicted in FIG. 18.

### Example VIII

### Use of Wild-Type Yeast Strains to Produce Optically Active 3-Chlorostyrene Epoxide and 3-Chlorophenyl Ethanediol from (±)-3-Chlorostyrene Epoxide

Enantiomeric excesses (ee; calculated as described in Example III) of the remaining epoxide enantiomer obtained after incubation of the different yeast strains (tested in this Example as Samples 39-132) with 3-chlorostyrene epoxide are shown in Table 3.

Reaction conditions: 20 mM 3-chloroSEO, 20% cells (wet w/v), 25° C. Reaction time=300 minutes. Positive ee values indicate that the [S]-epoxide was in excess (i.e., that the relevant yeast strain was enantioselective for the (R) enantiomer), while negative ee values indicate opposite enantioselectivity.

TABLE 3
Enantiomeric excesses (ee) of the remaining (S)-epoxide enantiomer obtained
after incubation of the different yeast strains with 3-chlorostyrene epoxide

| Sample | Internal | | Culture | ee (%) |
|---|---|---|---|---|
| no. | strain no. | Strain | collection no. | (S) |
| 39 | Jen-25 | Bullera dendrophila | NCYC 3152 | 21.0 |
| 40 | Jen-26 | Bullera dendrophila | NCYC 3208 | 6.1 |
| 41 | 708 | Candida rugosa | NCYC 3155 | 7.0 |
| 42 | Jen-17 | Cryptococcus albidus | UOFS Y-0223 | 5.9 |
| 43 | Jen-2 | Cryptococcus albidus | NCYC 3156 | −3.6 |
| 44 | Jen-15 | Cryptococcus hungaricus | NCYC 3159 | 11.2 |
| 45 | AB-24 | Cryptococcus laurentii | NCYC 3161 | 14.3 |
| 46 | AB-26 | Cryptococcus laurentii | UOFS Y-1885 | 13.5 |
| 47 | AB-32 | Cryptococcus laurentii | UOFS Y-1887 | 12.6 |
| 48 | AB-25 | Cryptococcus laurentii | UOFS Y-1884 | 10.7 |
| 49 | AB-33 | Cryptococcus laurentii | UOFS Y-1888 | 10.2 |
| 50 | Jen-12 | Cryptococcus laurentii | UOFS Y-0135 | 9.3 |
| 51 | AB-27 | Cryptococcus laurentii | UOFS Y-1886 | 5.6 |
| 52 | AB-50 | Cryptococcus podzolicus | UOFS Y-1883 | 20.1 |
| 53 | AB-28 | Cryptococcus podzolicus | UOFS Y-1889 | 11.7 |
| 54 | AB-30 | Cryptococcus podzolicus | UOFS Y-1904 | 11.4 |
| 55 | AB-39 | Cryptococcus podzolicus | UOFS Y-1912 | 8.9 |
| 56 | AB-29 | Cryptococcus podzolicus | UOFS Y-1897 | 6.4 |
| 57 | AB-58 | Cryptococcus podzolicus | NCYC 3164 | 6.1 |
| 58 | AB-34 | Cryptococcus podzolicus | UOFS Y-1890 | 4.1 |
| 59 | 113 | Debaryomyces hansenii | UOFS Y-0058 | 4.5 |
| 60 | 520 | Pichia finlandica | NCYC 3173 | −41.3 |
| 61 | 674 | Pichia guillermondii | UOFS Y-1030 | 5.4 |
| 62 | 675 | Pichia guillermondii | UOFS Y-1033 | 3.7 |
| 63 | 706 | Pichia guillermondii | UOFS Y-0057 | 2.6 |
| 64 | 112 | Pichia guillermondii | UOFS Y-0053 | 2.1 |
| 65 | 707 | Pichia guillermondii | NCYC 3174 | 1.8 |
| 66 | 28 | Pichia haplophila | UOFS Y-2136 | −3.8 |
| 67 | 673 | Pichia haplophila | NCYC 3177 | −19.8 |
| 68 | 692 | Rhodosporidium paludigenum | NCYC 3179 | 10.1 |
| 69 | Car-052 | Rhodosporidium toruloides | UOFS Y-2230 | 83.1 |
| 70 | Car-020 | Rhodosporidium toruloides | UOFS Y-2226 | 74.9 |
| 71 | Car-059 | Rhodosporidium toruloides | UOFS Y-2231 | 72.6 |
| 72 | AB 1 | Rhodosporidium toruloides | NCYC 3181 | 72.0 |
| 73 | Car-126 | Rhodosporidium toruloides | UOFS Y-2251 | 70.9 |
| 74 | Car-118 | Rhodosporidium toruloides | NCYC 3182 | 65.2 |
| 75 | Car-120 | Rhodosporidium toruloides | UOFS Y-2249 | 62.6 |
| 76 | Car-108 | Rhodosporidium toruloides | UOFS Y-2247 | 61.8 |
| 77 | Car-204 | Rhodosporidium toruloides | UOFS Y-2257 | 60.8 |
| 78 | Car-078 | Rhodosporidium toruloides | UOFS Y-2240 | 59.1 |
| 79 | Car-134 | Rhodosporidium toruloides | UOFS Y-2253 | 58.4 |
| 80 | Car-077 | Rhodosporidium toruloides | UOFS Y-2239 | 56.9 |
| 81 | Car-038 | Rhodosporidium toruloides | UOFS Y-2228 | 55.6 |
| 82 | Car-006 | Rhodosporidium toruloides | UOFS Y-2223 | 48.5 |
| 83 | Car-076 | Rhodosporidium toruloides | UOFS Y-2238 | 47.0 |
| 84 | Car-092 | Rhodosporidium toruloides | UOFS Y-2241 | 46.6 |
| 85 | Car-067 | Rhodosporidium toruloides | UOFS Y-2236 | 45.1 |
| 86 | Car-093 | Rhodosporidium toruloides | UOFS Y-2242 | 44.7 |
| 87 | Car-003 | Rhodosporidium toruloides | UOFS Y-2222 | 42.4 |
| 88 | Car-142 | Rhodosporidium toruloides | UOFS Y-2255 | 40.9 |
| 89 | Car-210 | Rhodosporidium toruloides | UOFS Y-2261 | 38.3 |
| 90 | Car-121 | Rhodosporidium toruloides | UOFS Y-2250 | 36.6 |
| 91 | AB 2 | Rhodosporidium toruloides | UOFS Y-0518 | 33.9 |
| 92 | Car-070 | Rhodosporidium toruloides | UOFS Y-2237 | 33.8 |
| 93 | Car-094 | Rhodosporidium toruloides | UOFS Y-2243 | 32.2 |
| 94 | Car-103 | Rhodosporidium toruloides | UOFS Y-2246 | 30.8 |
| 95 | Car-205 A | Rhodosporidium toruloides | UOFS Y-2258 | 21.8 |
| 96 | Car-131 | Rhodosporidium toruloides | UOFS Y-2252 | 21.4 |
| 97 | Car-100 | Rhodosporidium toruloides | UOFS Y-2245 | 20.8 |
| 98 | Car-209 | Rhodosporidium toruloides | UOFS Y-2260 | 15.6 |
| 99 | 46 | Rhodosporidium toruloides | UOFS Y-0471 | 5.2 |
| 100 | EP-230 | Rhodotorula aurantiaca | NCYC 3185 | 62.9 |
| 101 | 50 | Rhodotorula glutinis | NCYC 3186 | 89.6 |
| 102 | Car-62 | Rhodotorula glutinis | UOFS Y-2234 | 64.5 |
| 103 | Car-75 | Rhodotorula glutinis | UOFS Y-2265 | 49.7 |
| 104 | 713 | Rhodotorula glutinis | UOFS Y-0489 | 43.6 |
| 105 | Car-61 | Rhodotorula glutinis | UOFS Y-2233 | 43.3 |
| 106 | Car-60 | Rhodotorula glutinis | UOFS Y-2232 | 42.6 |
| 107 | Car-66 | Rhodotorula glutinis | UOFS Y-2235 | 41.7 |
| 108 | Car-22 | Rhodotorula glutinis | UOFS Y-2227 | 24.8 |
| 109 | 680 | Rhodotorula glutinis | UOFS Y-0459 | 19.6 |
| 110 | AB 6 | Rhodotorula glutinis | UOFS Y-0513 | 12.0 |
| 111 | 714 | Rhodotorula minuta | NCYC 3187 | 32.8 |
| 112 | 712 | Rhodotorula minuta | UOFS Y-0835 | 8.2 |
| 113 | 165 | Rhodotorula sp. | UOFS Y-2043 | 3.0 |
| 114 | Jen-31 | Sporidiobolus salmonicolor | NCYC 3196 | 10.2 |
| 115 | Jen-32 | Sporidiobolus salmonicolor | NCYC 3195 | 6.8 |
| 116 | Jen-30 | Sporobolomyces holsaticus | NCYC 3198 | 15.4 |
| 117 | Jen-29 | Sporobolomyces roseus | NCYC 3197 | 15.6 |
| 118 | Jen-28 | Sporobolomyces tsugae | NCYC 3199 | −6.5 |
| 119 | 20 | Trichosporon cutaneum var. cutaneum | NCYC 3201 | −3.5 |
| 120 | 21 | Trichosporon cutaneum var. cutaneum | UOFS Y-0063 | −13.3 |
| 121 | 19 | Trichosporon ovoides | NCYC 3207 | −34.4 |
| 122 | 231 | Trichosporon sp. | UOFS Y-0533 | 2.3 |
| 123 | Car-205B | Trichosporon mantevideense | NCYC 3225 | −11.6 |
| 124 | Jen-39 | Yarrowia lipolytica | UOFS Y-1698 | −4.7 |
| 125 | Jen-38 | Yarrowia lipolytica | UOFS Y-0809 | −4.9 |
| 126 | Jen-33 | Yarrowia lipolytica | UOFS Y-0164 | −6.8 |
| 127 | Jen-41 | Yarrowia lipolytica | UOFS Y-1571 | −6.9 |
| 128 | Jen 46 | Yarrowia lipolytica | NCYC 3229 | −8.2 |
| 129 | Jen-43 | Yarrowia lipolytica | UOFS Y-1699 | −8.7 |
| 130 | Jen-37 | Yarrowia lipolytica | UOFS Y-0097 | −9.8 |
| 131 | Jen-46 | Yarrowia lipolytica | UOFS Y-1701 | −10.6 |
| 132 | Jen-48 | Yarrowia lipolytica | UOFS Y-1700 | −25.4 |

(negative ee values denote opposite enantioselectivity)

All the yeast strains referred to in this and the following examples are kept and maintained at the University of the Orange Free State (UOFS), Department of Microbial, Biochemical and Food Biotechnology, Faculty of Natural and Agricultural Sciences, P.O. Box 339, Bloemfontein 9300, South Africa (Tel+27 51 401 2396, Fax+27 51444 3219) and are readily identified by the yeast species and culture collection number as indicated. Representative examples of strains belonging to the different species have been deposited under the Budapest Treaty at National Collection of Yeast Cultures (NCYC), Institute of Food Research Norwich Research Park Colney, Norwich NR47UA, U.K. (Tel: +44-(0)1603-255274 Fax: +44-(0)1603-458-414 Email: ncyc@bbsrc.ac.uk) and are readily identified by the yeast species and culture collection accession number as indicated. The samples deposited with the NCYC are taken from the same deposit maintained by the South African Council for Scientific and Industrial Research (CSIR) since prior to the filing date of this application. The deposits will be maintained without restriction in the NCYC depository for a period of 30 years, or 5 years after the most recent request, or for the effective life of the patent, whichever is longer, and will be replaced if the deposit becomes non-viable during that period. Samples of the yeast strains not deposited at NCYC will be made available upon request on the same basis and conditions of the Budapest Treaty.

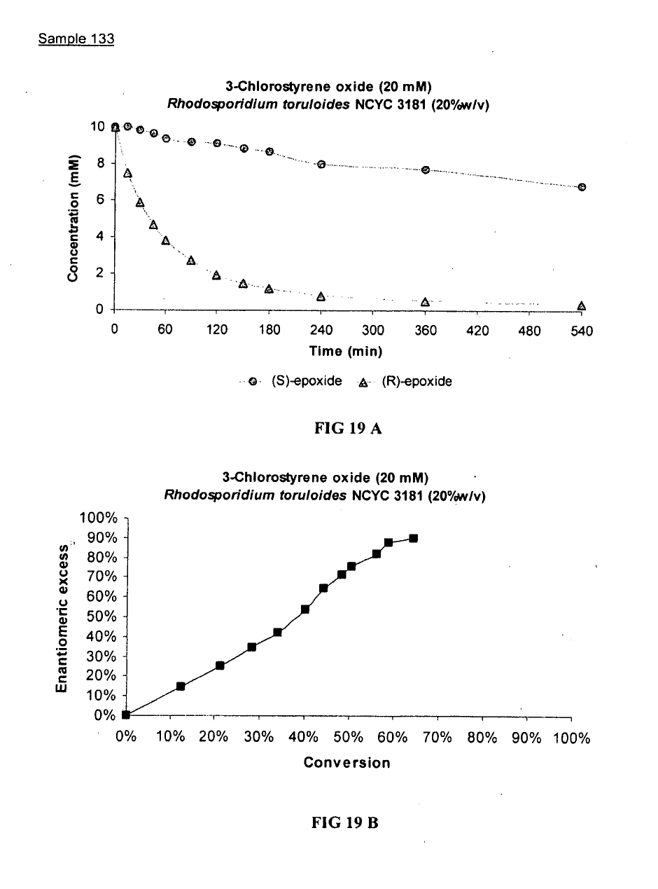

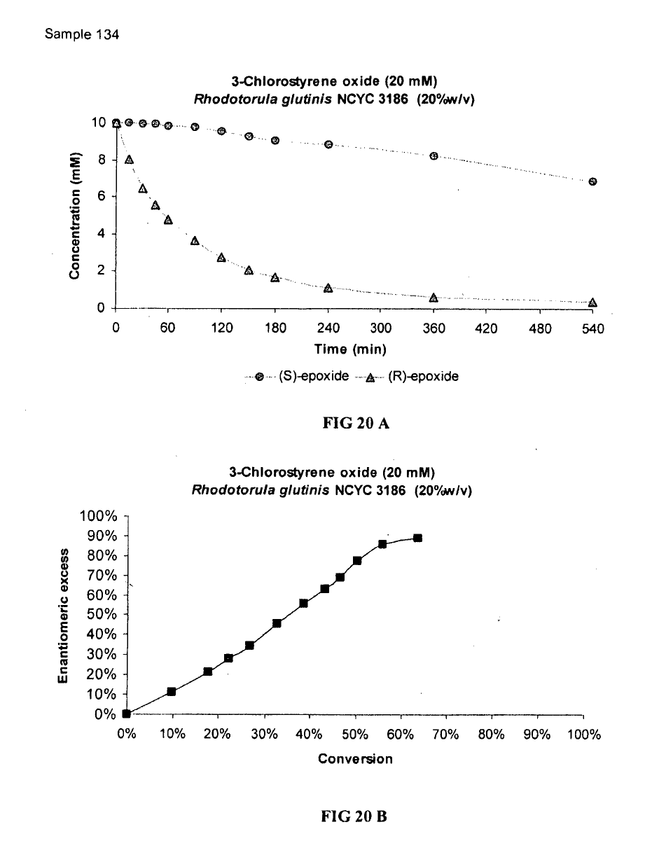

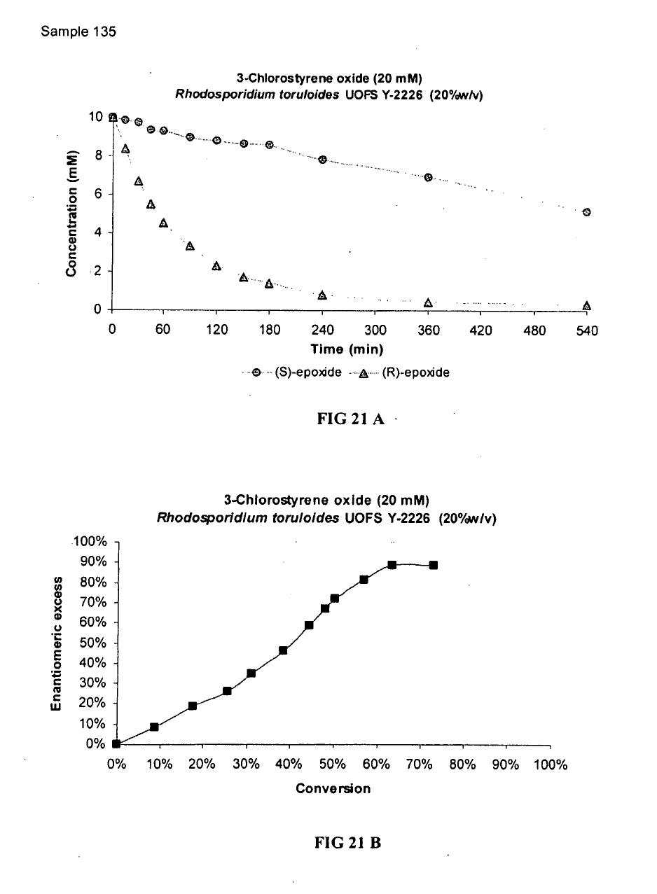

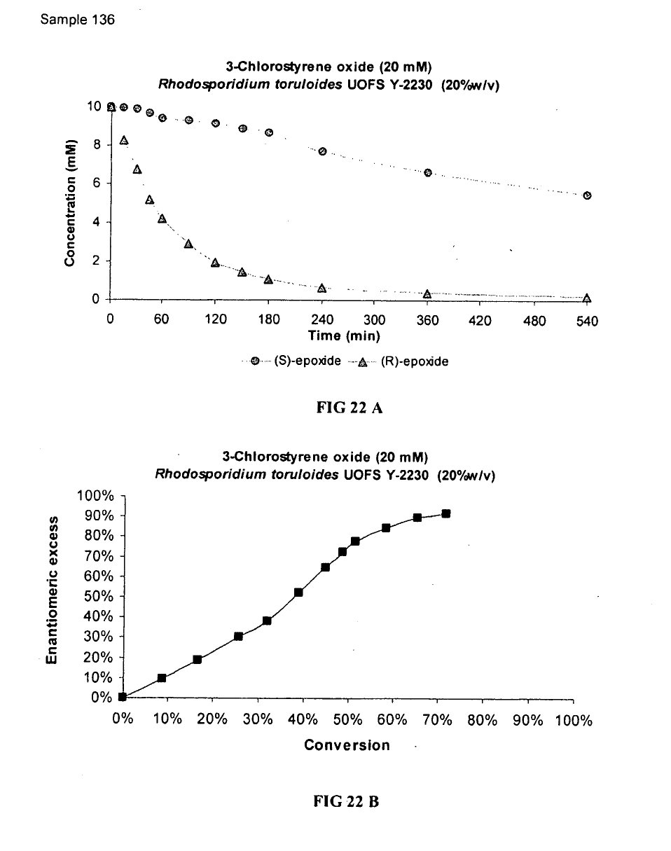

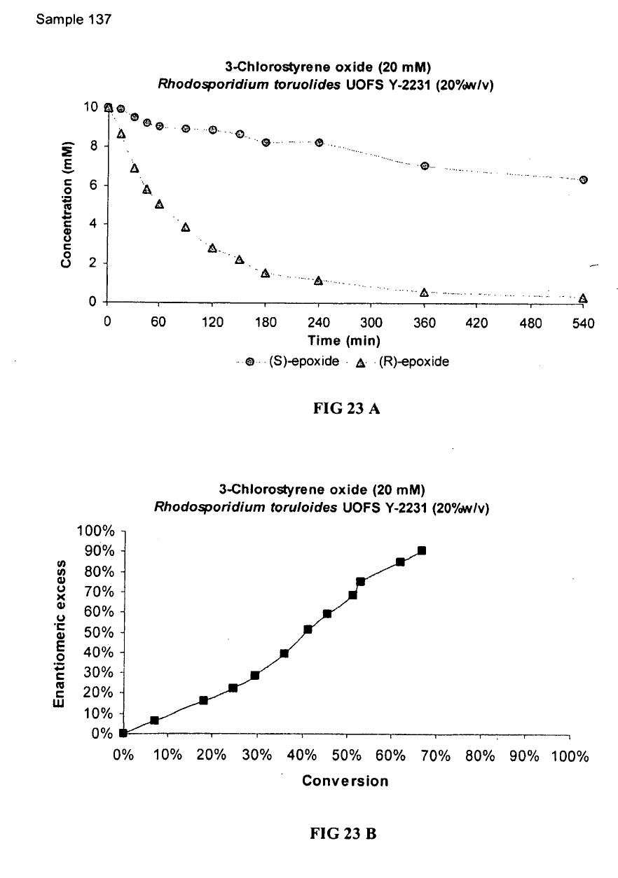

Various wild-type yeast strains selected from Table 3 were used (as Samples 133-137 in this example) to produce to produce optically active epoxides and vicinal diols from 3-chloroSEO. For each Sample, two graphs are provided (FIGS. 19-23). The first graph (panel A) shows the change in concentrations of the epoxide enantiomers with time, while the second graph (panel B) shows the enantiomeric excess of the epoxide at different conversions. The yield of the optically active epoxide that can be obtained at a particular enantiomeric purity can be obtained from these graphs.

### Example IX

### Production of Optically Active (S)-3-Chlorostyrene Epoxide and (R)-3-chlorophenylethanediol using Yeast Host Cells Transformed with the Epoxide Hydrolase Genes from Selected Wild Type Yeast Strains

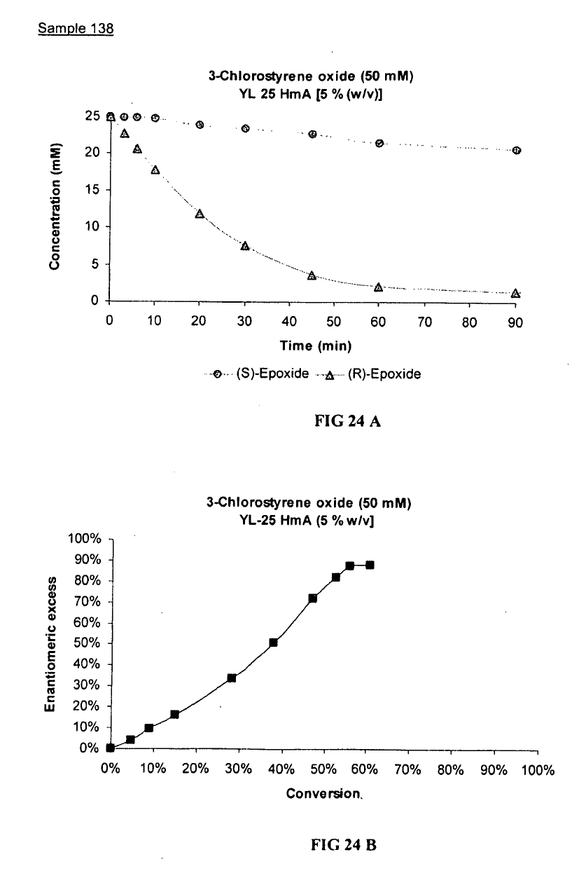

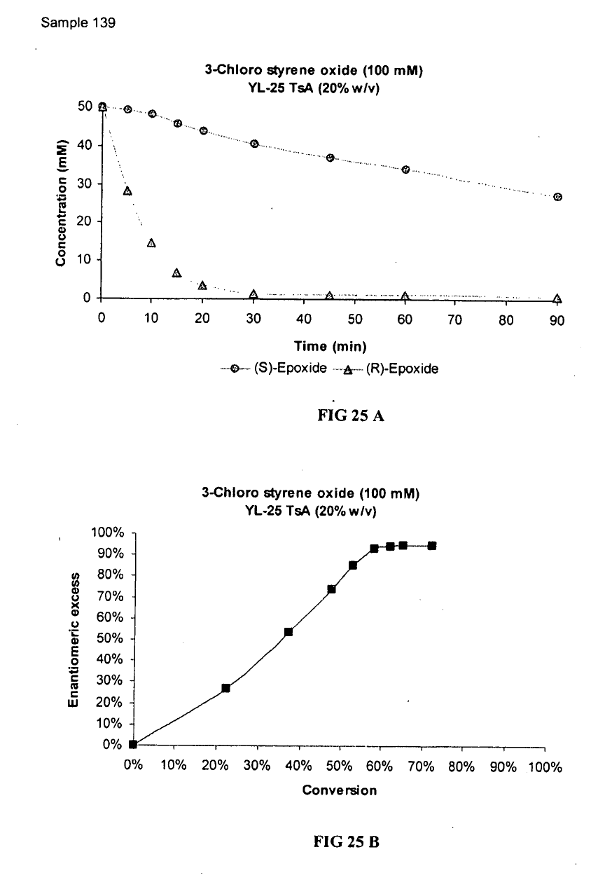

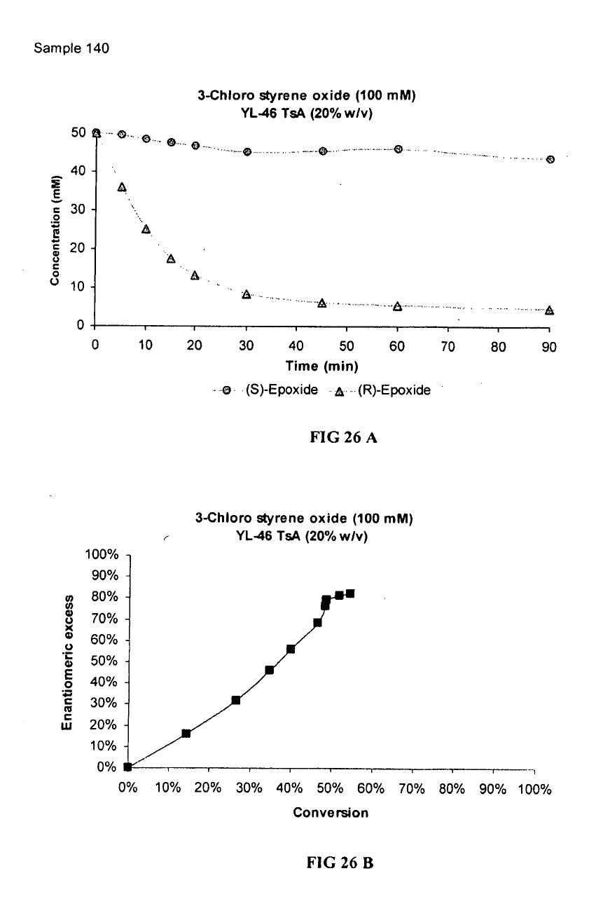

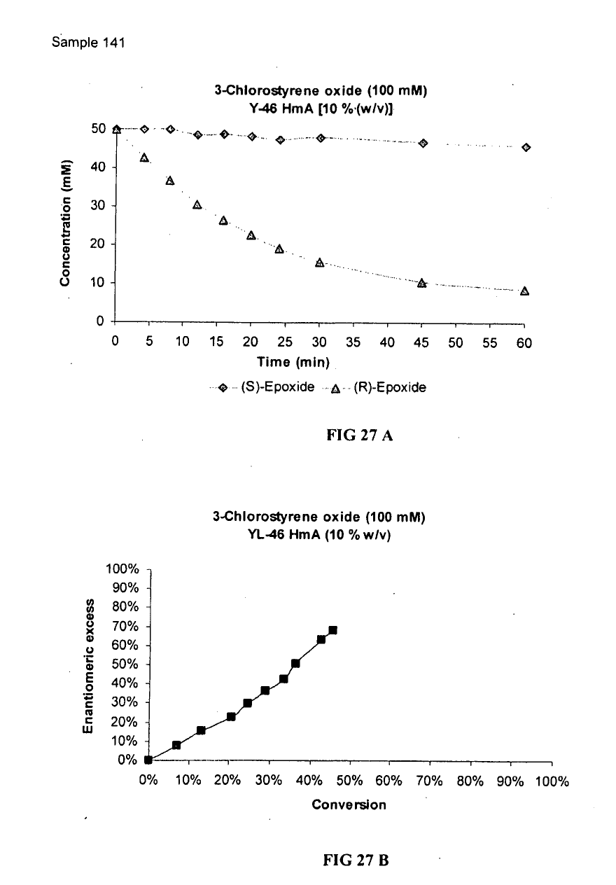

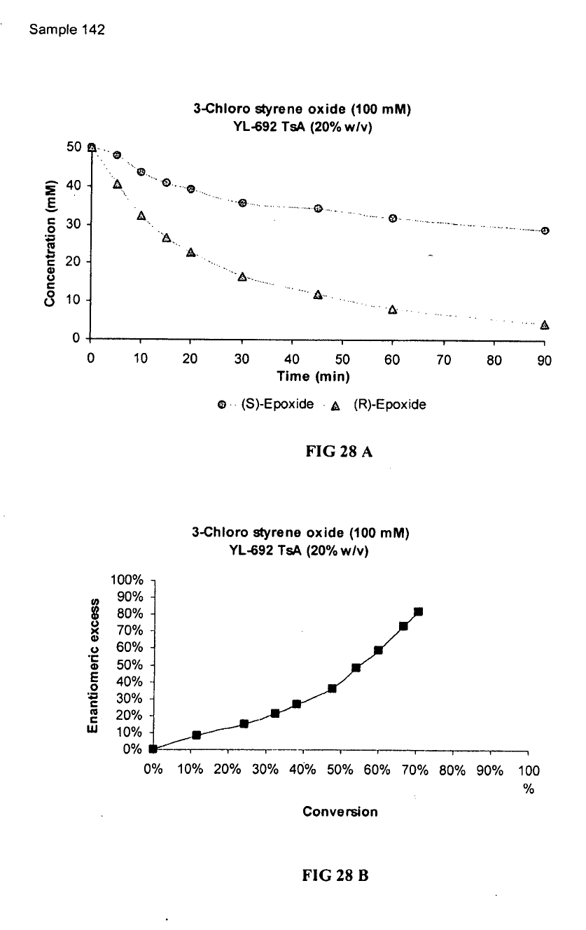

FIGS. 24-28 show the hydrolysis of (±)3-chloroSEO by recombinant yeast strains (tested in this example as Samples 138-142) expressing, under control of different promoters, exogenous epoxide hydrolases from selected wild-type yeast strains to produce (S)-3-chloroSEO and (R)-3-chloroPED. The first graph (panel A) shows the change in concentrations of the epoxide enantiomers with time, while the second graph (panel B) shows the enantiomeric excess of the residual epoxide at different conversions. The yield of the optically active epoxide that can be obtained at a particular enantiomeric purity can be obtained from these graphs.

### Example X

### Production of Optically Active (S)-2-Chlorostyrene Epoxide and (R)-2-chlorophenylethanediol using Yeast Host Cells Transformed with the Epoxide Hydrolase Genes from Selected Wild Type Yeast Strains

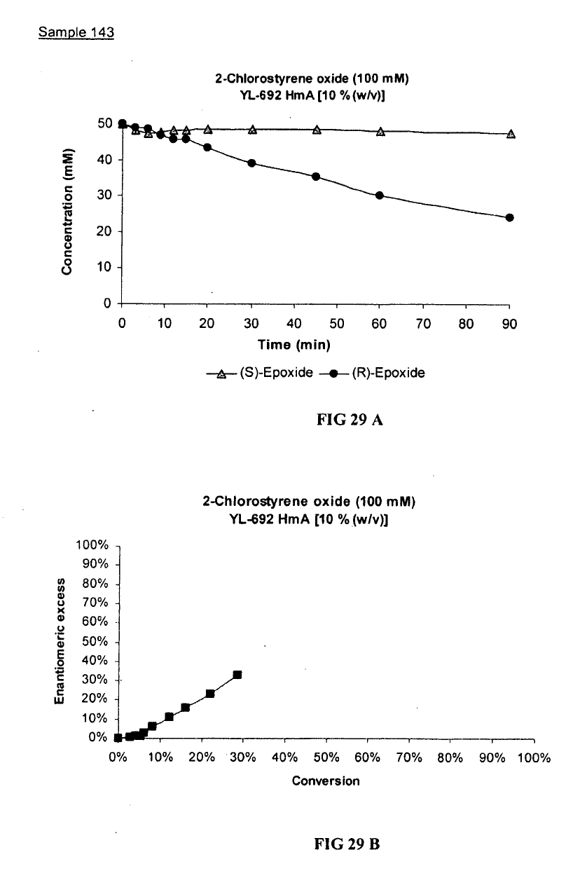

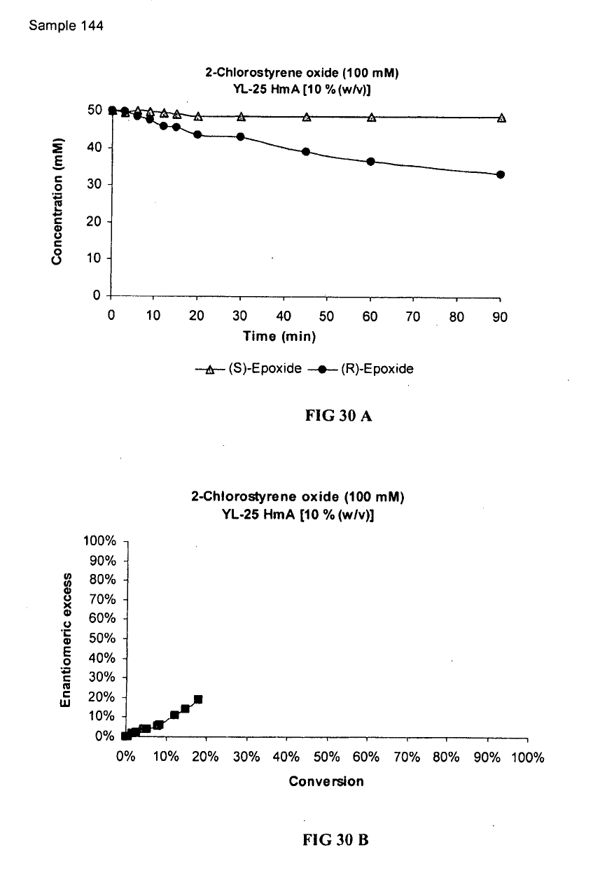

FIGS. 29-30 show the hydrolysis of (±)2-chloroSEO by recombinant yeast strains (tested in this example as Samples 143 and 144) expressing, under control of Hp4d promoter, exogenous epoxide hydrolases from selected wild-type yeast strains to produce (S)-2-chloroSEO and (R)-2-chloroPED. The first graph (panel A) shows the change in concentrations of the epoxide enantiomers with time, while the second graph (panel B) shows the enantiomeric excess of the residual epoxide at different conversions. The yield of the optically active epoxide that can be obtained at a particular enantiomeric purity can be obtained from these graphs.

### Example XI

### Production of Optically Active (S)-4-Chlorostyrene Epoxide and (R)-4-chlorophenylethanediol using Yeast Host Cells Transformed with the Epoxide Hydrolase Genes from Selected Wild Type Yeast Strains

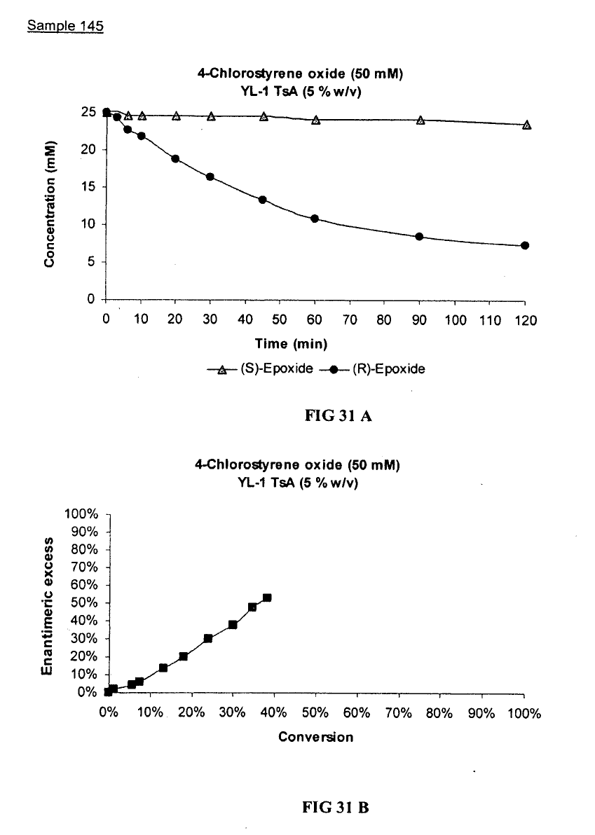

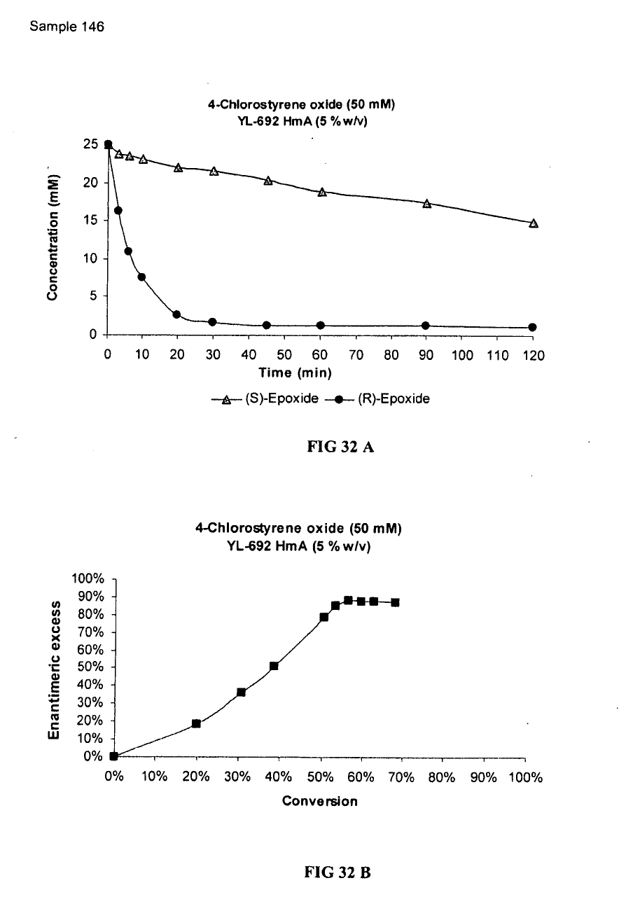

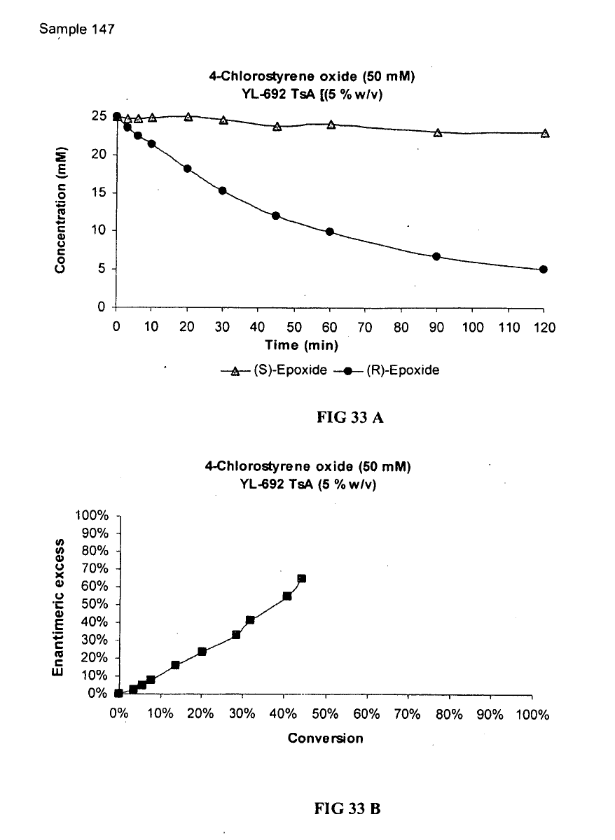

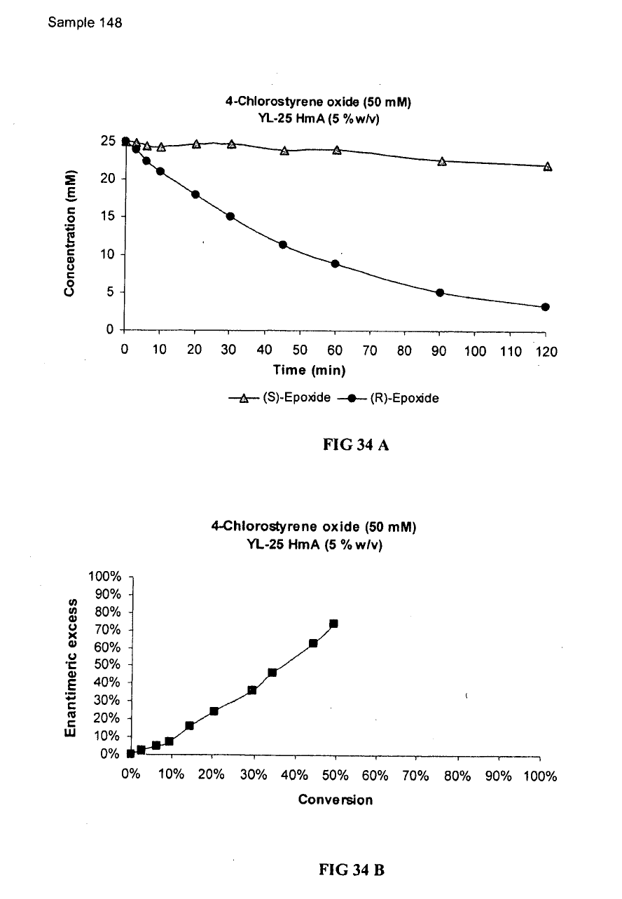

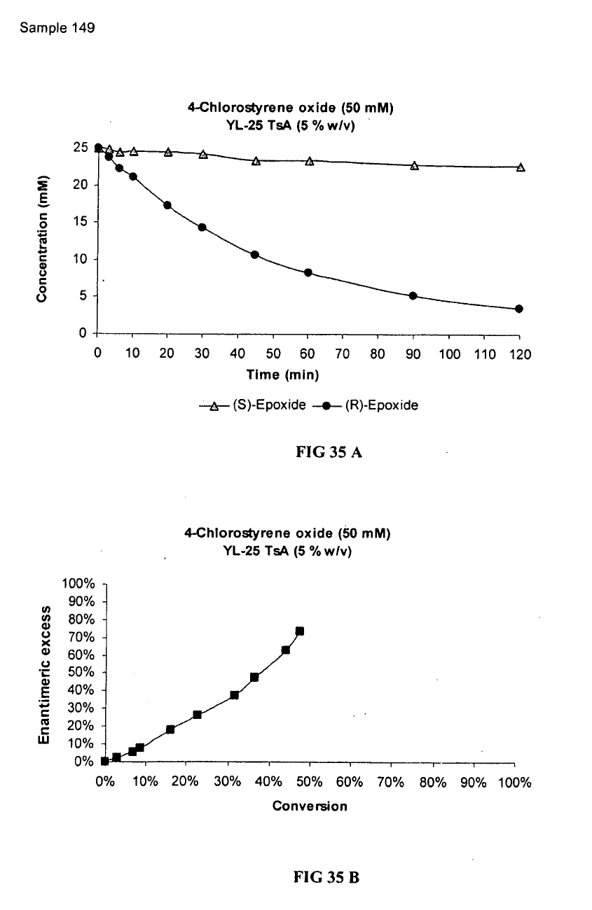

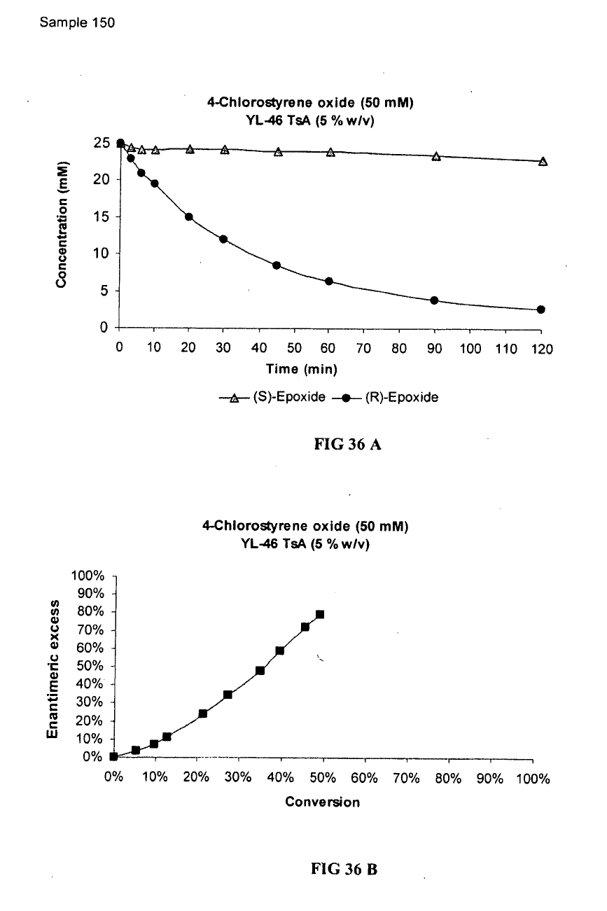

FIGS. 31-36 show the hydrolysis of (±)-4-chloroSEO by recombinant yeast strains (tested in this example as Samples 145-150) expressing, under control of different promoters, exogenous epoxide hydrolases from selected wild-type yeast strains to produce (S)-4-chloroSEO and (R)-4-chloroPED. The first graph (panel A) shows the change in concentrations of the epoxide enantiomers with time, while the second graph (panel B) shows the enantiomeric excess of the residual epoxide at different conversions. The yield of the optically active epoxide that can be obtained at a particular enantiomeric purity can be obtained from these graphs.

### Example XII

### Production of Optically Active (S or R)-2, -, 3-, or 4-Nitrostyrene Epoxide and (R or S)-2-, 3-, or 4-Nitrophenylethanediol using Selected Wild-Type Yeast Strains or Yeast Host Cells Transformed with the Epoxide Hydrolase Genes from Selected Wild Type Yeast Strains

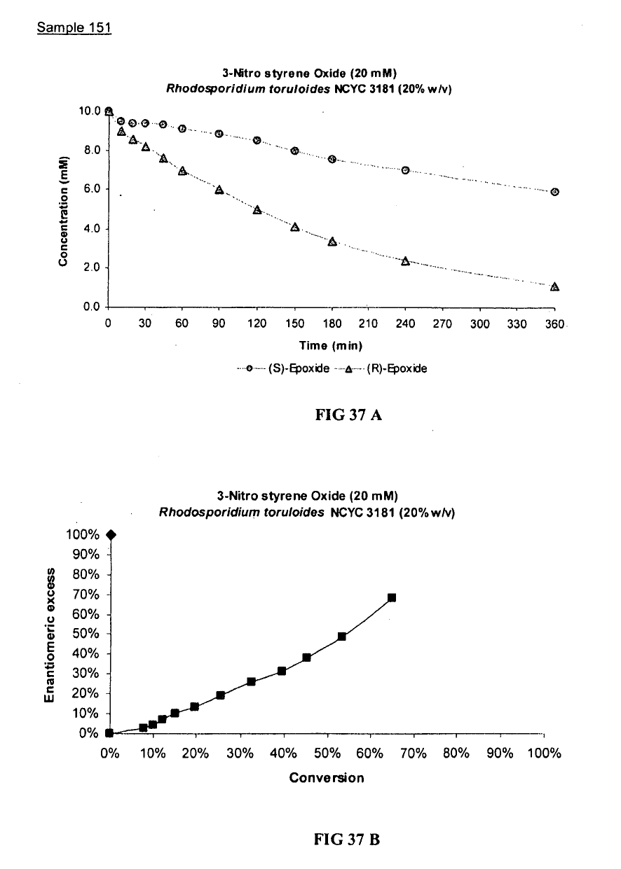

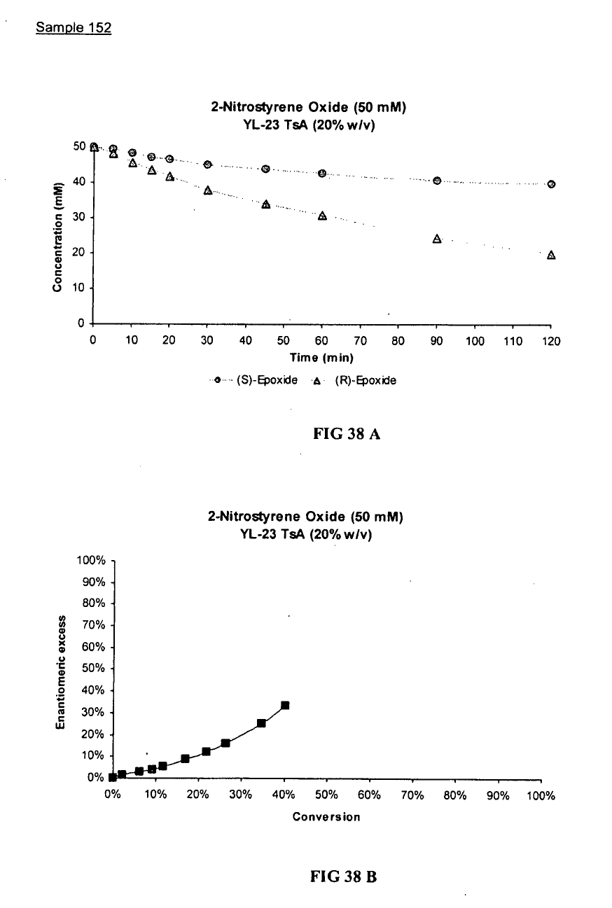

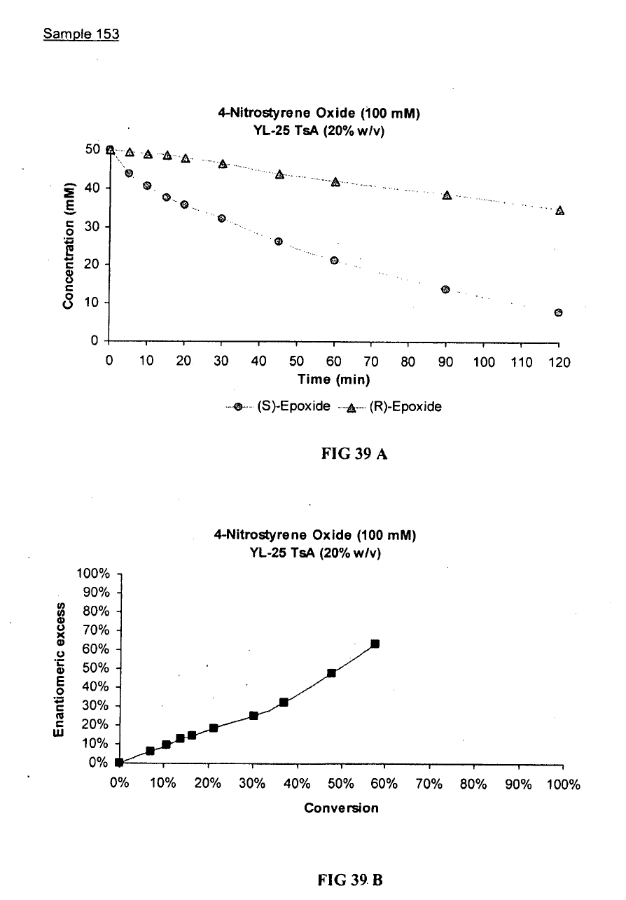

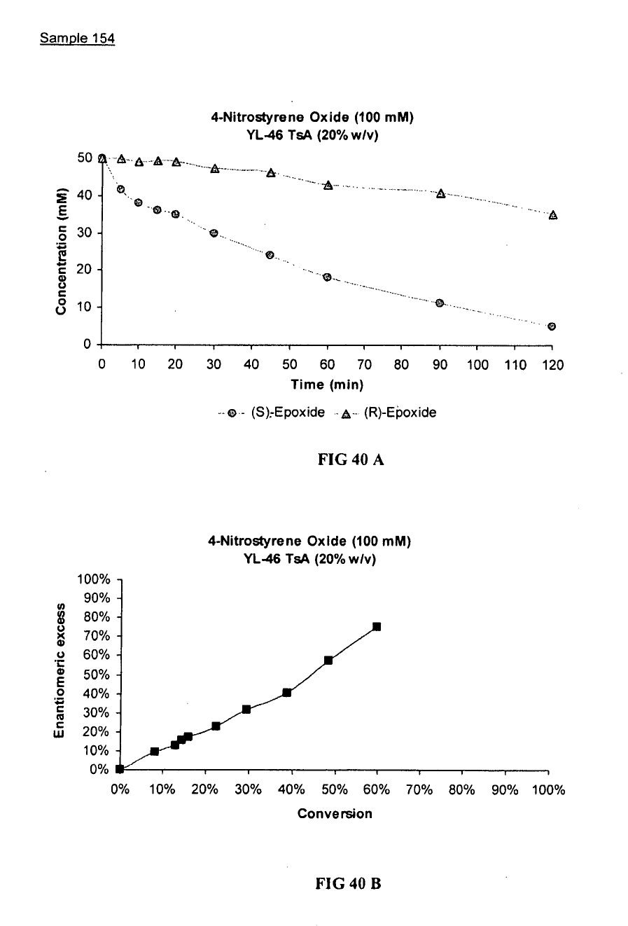

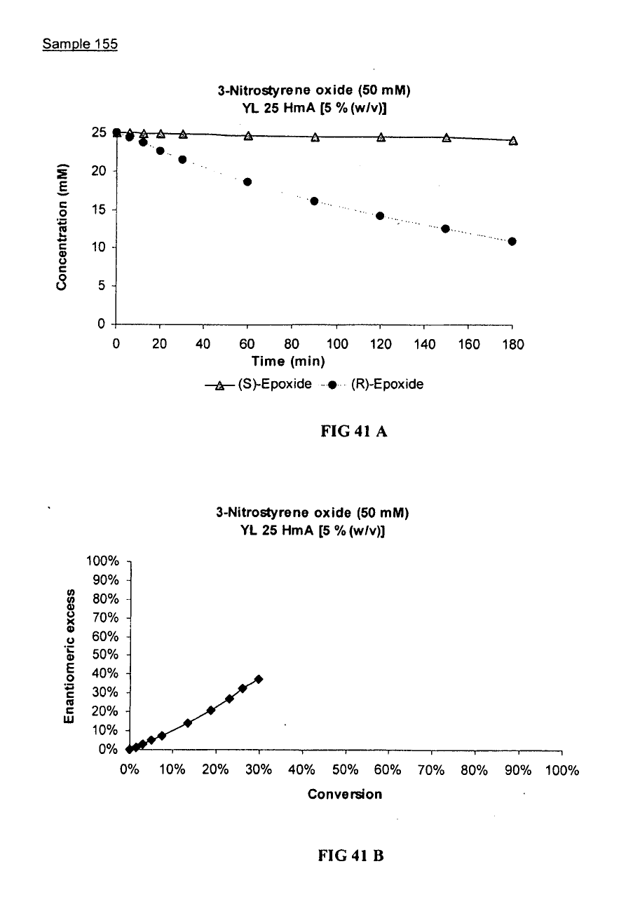

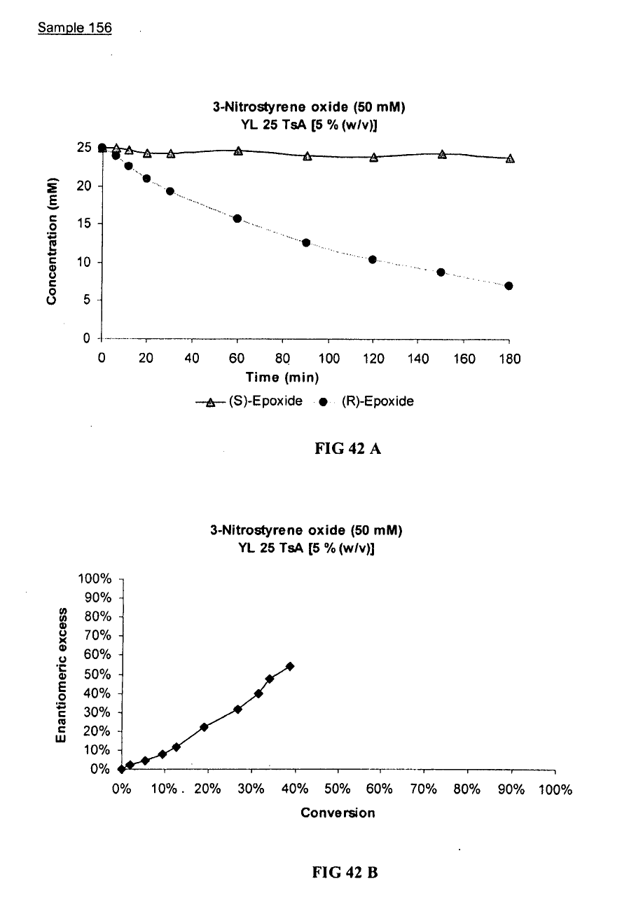

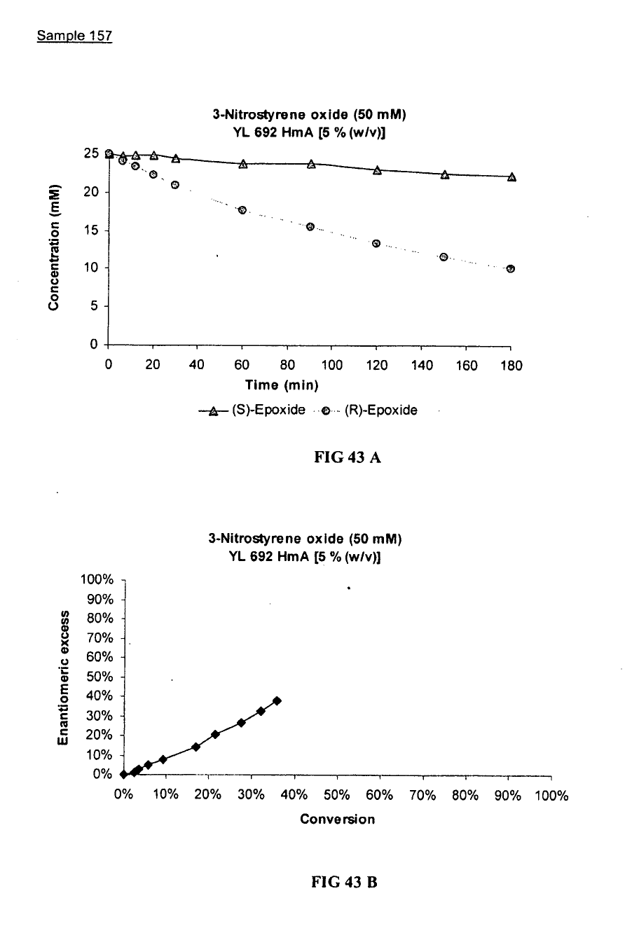

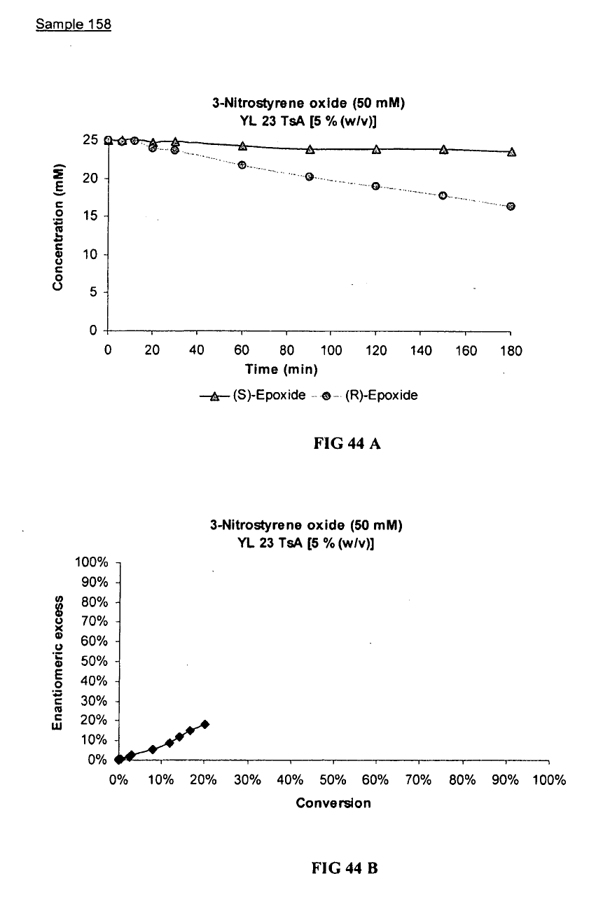

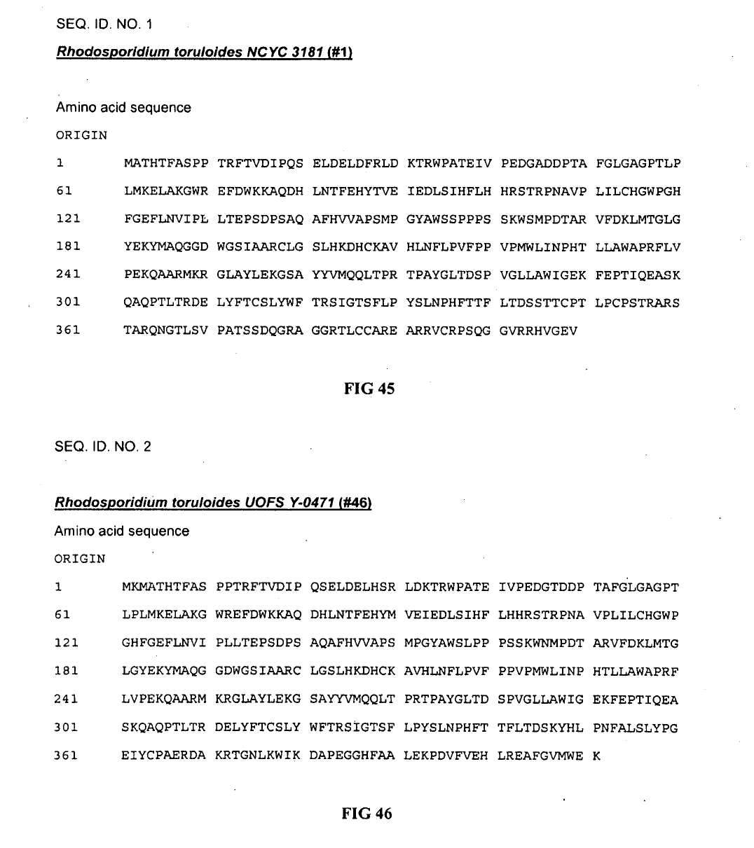

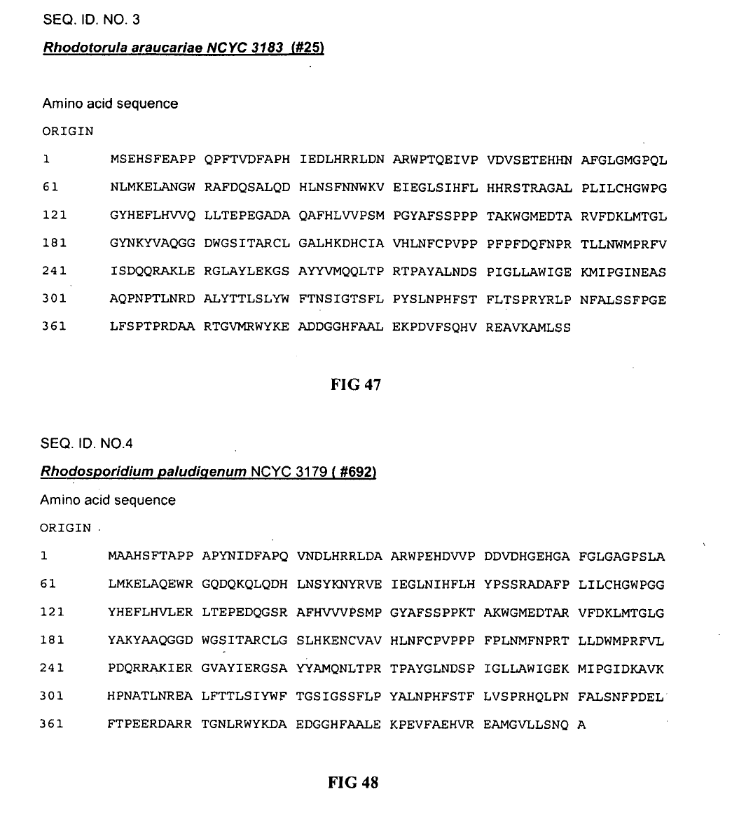

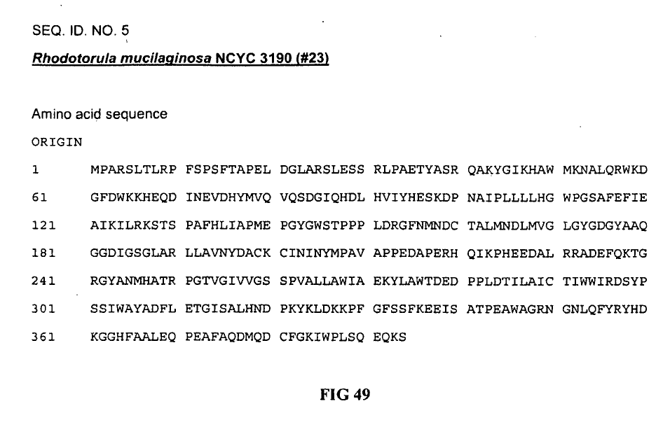

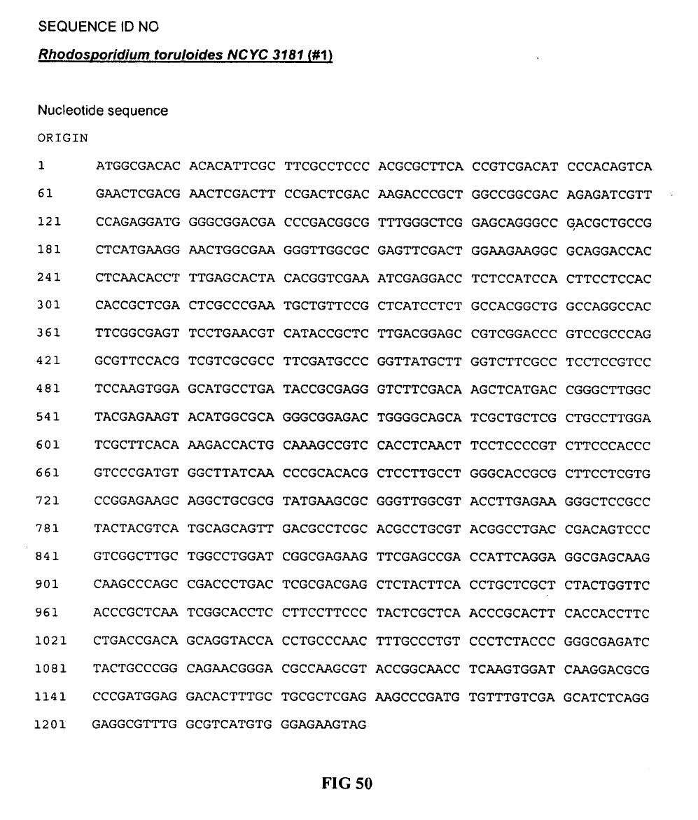

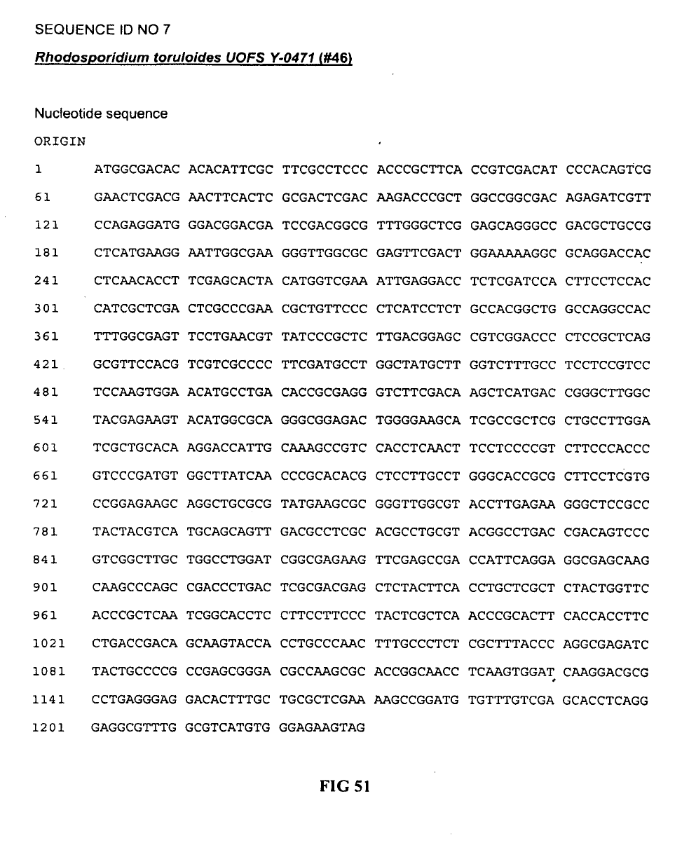

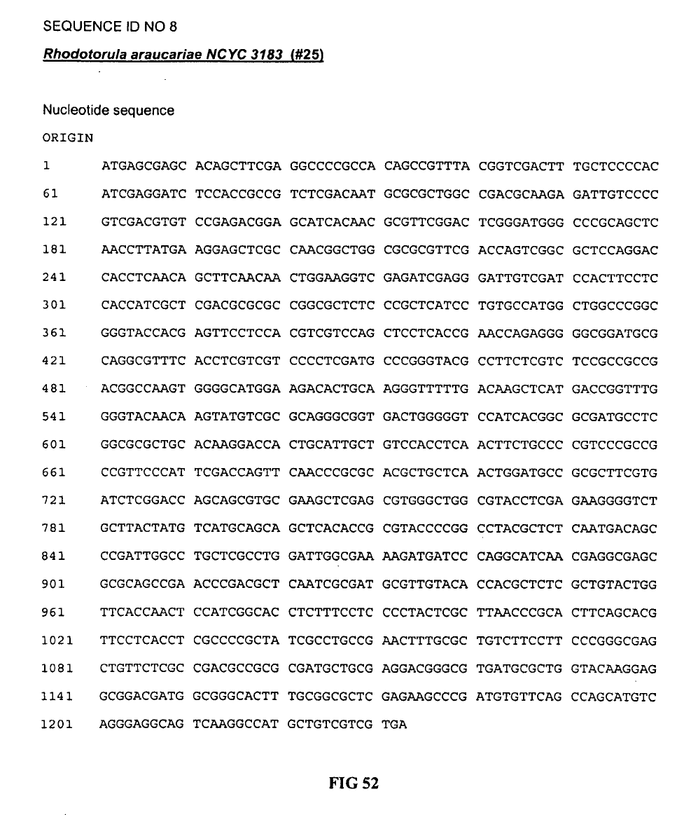

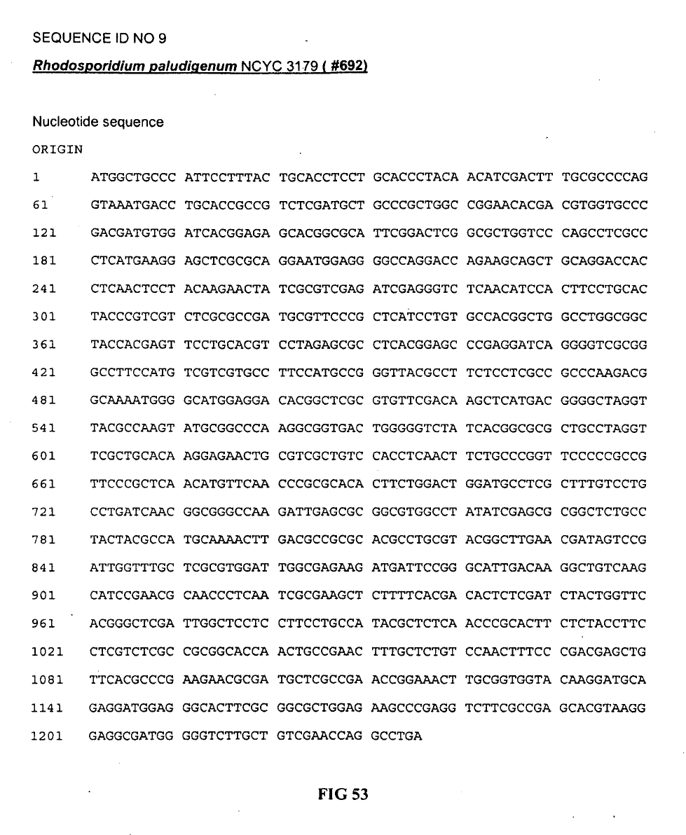

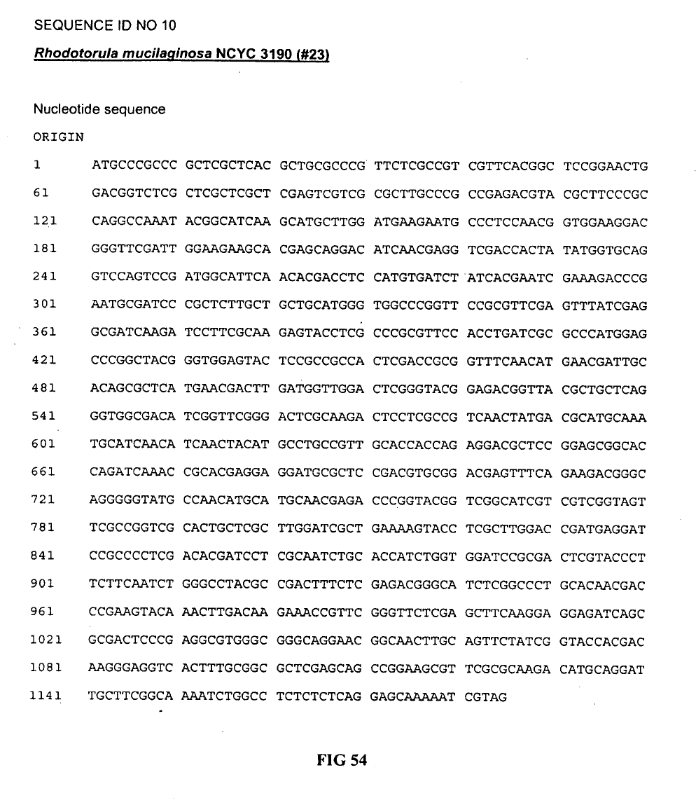

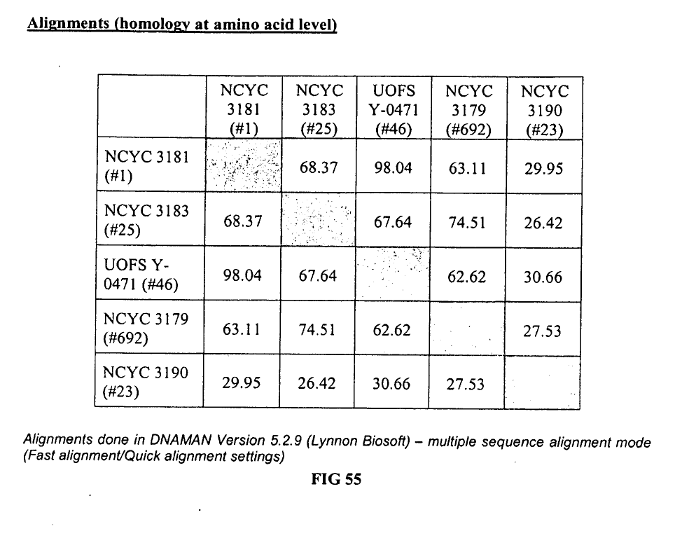

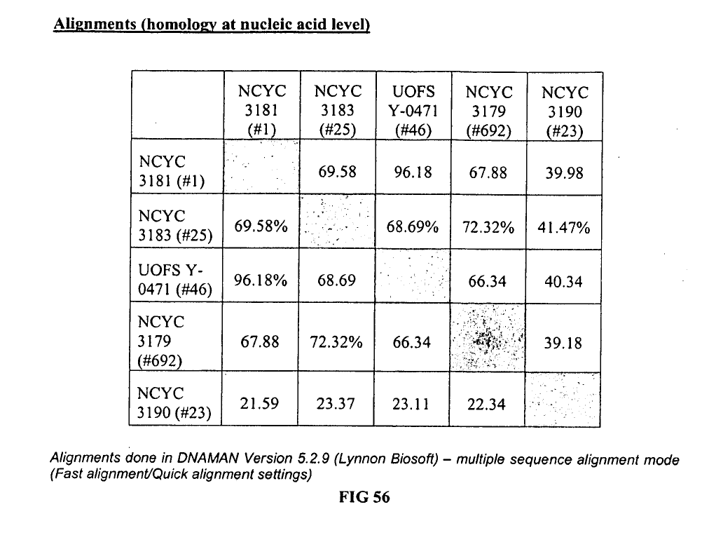

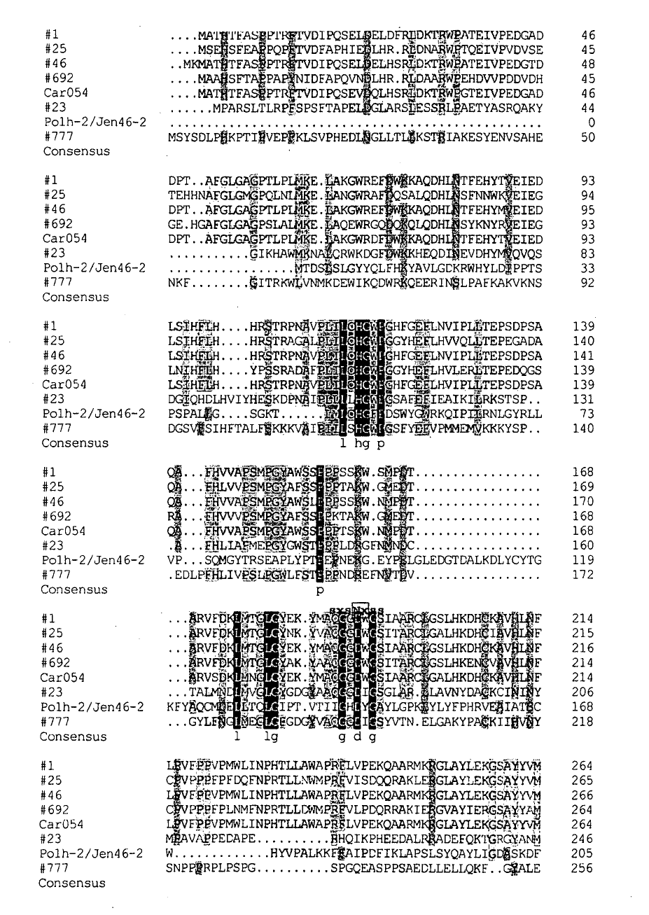

FIGS. 37-44 show the hydrolysis of (±)-4-nitroSEO by wild-type yeast strains or recombinant yeast strains (tested in this example as Samples 151-158) expressing, under control of different promoters, exogenous epoxide hydrolases from selected wild-type yeast strains to produce (S or R)-2-, 3-, or -4-nitroSEO and (R or S)-2-, 3-, or -4-nitroPED. The first graph (panel A) shows the change in concentrations of the epoxide enantiomers with time, while the second graph (panel B) shows the enantiomeric excess of the residual epoxide at different conversions. The yield of the optically active epoxide that can be obtained at a particular enantiomeric purity can be obtained from these graphs.

### REFERENCES

Harada, H., Hirokawa, Y., Suzuki, K., Hiyama, Y., Oue, M. et al., (2003). Novel and potent human and rat β₃-adrenergic receptor agonists containing substituted 3-indolylalkylamines. Bioorganic and Medicinal Chemistry Letters 13: 1301-1305.
Manoj, K. M., Archelas, A., Baratti, J., Furstoss, R. (2001). Microbiological transformations. Part 45: A green chemistry preparative scale synthesis of enantiopure building blocks of Eliprodil. Tetrahedron 57: 695-701.
Monterde, M. I., Lombard, M., Archelas, A., Cronin, A., Arand, M., Furstoss, R. (2004). Enzymatic transformations. Part 58: Enantioconvergent biohydrolysis of styrene oxide derivatives catalysed by the Solanum tuberosum epoxide hydrolase. Tetrahedron: Asymmetry 15: 2801-2805.
Nicaud J-M, Madzak C, Van den Broek P, Gysler C, Duboc P, Niederberger P, Gaillardin C. (2002) Protein expression and secretion in the yeast Yarrowia lipolytica. FEMS Yeast Research 2, 371-279.
Pedragosa-Moreau, S., Archelas, A. and Furstoss, R. (1996) Microbiological Transformations 32. Use of epoxide hydrolase mediated biohydrolysis as a way to enantiopure epoxides and vicinal diols—Application to substituted styrene oxide derivatives. Tetrahedron 52: 4593-4606.
Xuan J-W, Fournier P, Gaillardin C. (1988) Cloning of the LYS gene encoding saccharopine dehydrogenase from the yeast Yarrowia lipolytica by target integration. Current Genetics 14, 15-21.

A number of embodiments of the invention have been described. Nevertheless, it will be understood that various modifications can be made without departing from the spirit and scope of the invention. Accordingly, other embodiments are within the scope of the following claims.

1. A process for obtaining an optically active epoxide or an optically active vicinal diol, which process includes the steps of:
providing an enantiomeric mixture of a styrene epoxide;
creating a reaction mixture by adding to the enantiomeric mixture a polypeptide, or a functional fragment thereof, having enantioselective styrene epoxide hydrolase activity, the polypeptide being a polypeptide encoded by a gene of a yeast cell;
incubating the reaction mixture; and
recovering from the reaction mixture: (a) an enantiopure, or a substantially enantiopure, phenylethanediol; (b) an enantiopure, or a substantially enantiopure, styrene epoxide; or (c) an enantiopure, or a substantially enantiopure, phenylethanediol and an enantiopure, or a substantially enantiopure, styrene epoxide.

2. A process for obtaining an optically active epoxide or an optically active vicinal diol, which process includes the steps of:
providing an enantiomeric mixture of a styrene epoxide;
creating a reaction mixture by adding to the enantiomeric mixture a cell comprising a nucleic acid encoding, and capable of expressing, a polypeptide having enantioselective styrene epoxide hydrolase activity;
incubating the reaction mixture; and
recovering from the reaction mixture: (a) an enantiopure, or a substantially enantiopure, phenylethanediol; (b) an enantiopure, or a substantially enantiopure, styrene epoxide; or (c) an enantiopure, or a substantially enantiopure, phenylethanediol and an enantiopure, or a substantially enantiopure, styrene epoxide.

3. The process of claim 1, wherein the cell is a yeast cell.

4. The process of claim 1, wherein the polypeptide is encoded by an endogenous gene of the cell.

5. The process of claim 2, wherein the cell is a recombinant cell and the polypeptide is encoded by a nucleic acid sequence with which the cell is transformed.

6. The process of claim 5, wherein the nucleic acid sequence is a heterologous nucleic acid sequence.

7. The process of claim 5, wherein the nucleic acid sequence is a homologous nucleic acid sequence.

8. The process of claim 1, wherein the polypeptide is a full-length yeast epoxide hydrolase.

9. The process of claim 1, wherein the polypeptide is a functional fragment of yeast epoxide hydrolase.

10. The process of claim 1, wherein the process is carried out at a pH from 5 to 10.

11. The process of claim 1, wherein the process is carried out at a temperature of 0° C. to 70° C.

12. The process of claim 1, wherein the concentration of the styrene epoxide in the reaction matrix is at least equal to the soluble concentration of the styrene epoxide in water.

13. The process of claim 1, wherein the styrene epoxide of the enantiomeric mixture and the obtained optically active epoxide is a compound of the general formula (I) and the vicinal diol produced by the process is a compound of the general formula (II), 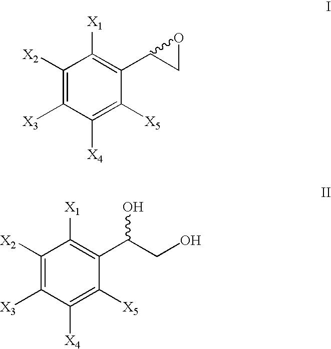
wherein,
X₁, X₂, X₃, X₄ and X₅ are, independently of each other, selected from: H, halogens, hydroxyl groups, mercapto groups, carboxylates, nitro groups, cyano groups, substituted or unsubstituted amino groups, amide groups, alkoxy groups, alkenyloxy groups, aryloxy groups, aryl alkyloxy groups, alkylthio groups, alkoxycarbonyl groups, substituted or unsubstituted carbamoyl groups, acyl groups, substituted and unsubstituted alkyl groups; substituted and unsubstituted alkenyl groups; and substituted and unsubstituted aryl groups, wherein the number of substituents is one or more than one and wherein the substituents are the same or different; or
X₁ and X₂, or X₂ and X₃, or X₃ and X₄ or X₄ and X₅ together and independent are a substituted or unsubstituted aryl group selected from the group consisting of: phenyl; biphenyl; naphtyl; anthracenyl groups; and the like; or
X₁ and X₂, or X₂ and X₃, or X₃ and X₄, or X₄ and X₅ together and independent are a cycloalkyl group with 4 to 8 carbon atoms, wherein the cycloalkyl group is selected from the group consisting of: cyclobutyl-; cyclopentyl-; cyclohexyl-; cycloheptyl-; and cyclooctyl-groups, wherein the cycloalkyl group is unsubstituted or variably substituted at any position of the ring; or
X₁ and X₂, or X₂ and X₃, or X₃ and X₄, or X₄ and X₅ together and independent are a cycloalkenyl group with 4 to 8 carbon atoms, wherein the cycloalkenyl group is selected from the group consisting of: cyclobutenyl-; cyclopentenyl-; cyclohexenyl-; cycloheptenyl-; and cyclooctenyl-groups, wherein the cycloalkenyl group is unsubstituted or is variably be substituted at one or more positions in the ring; or
X₁ and X₂, or X₂ and X₃, or X₃ and X₄, or X₄ and X₅ together and independent are a heterocyclic group consisting of a 5- to 7-membered heterocyclic group containing a nitrogen atom, an oxygen atom; or a sulfur atom, wherein the heterocyclic group is selected from the group consisting of: furyl-; dihydrofuranyl-; tetrahydrofuranyl-; dioxolanyl-; oxazolyl-; dihydrooxazolyl-; oxazolidinyl-; isoxazolyl-; dihydroisoxazolyl-; isoxazolidinyl-; oxathiolanyl-; thienyl-; tetrahydrothienyl-; dithiolanyl-; thiazolyl-; dihydrothiazolyl-; thiazolidinyl-; isothiazolyl-; dihydroisothiazolyl-; isothiazolidinyl-; pyrrolyl-; dihydropyrrolyl-; pyrrolidinyl-; pyrazolyl-; dihydropyrazolyl-; pyrazolidinyl-; imidazolyl-; dihydroimidazolyl-; imidazolidinyl-; triazolyl-; dihydrotriazolyl-; triazolidinyl-; tetrazolyl-; dihydrotetrazolyl-; tetrazolidinyl-; pyridyl-; dihydropyridyl-; piperidinyl-; morpholinyl-; dioxanyl-; oxathianyl-; trioxanyl-; thiomorpholinyl-; pyridazinyl-; dihydropyridazinyl-; tetrahydropyridazinyl-; hexahydropyridazinyl-; pyrimidinyl-; dihydropyrimadinyl-; tetrahydropyrimadinyl-; hexahydropyrimadinyl-; pyrazinyl-; piperazinyl-; pyranyl-; dihydropyranyl-; tetrahydropyranyl-; thiopyranyl-; dihydrothiopyranyl-; tetrahydrothiopyranyl-; dithianyl-; purinyl-; pyrimidinyl-; pyrrolizinyl-; pyrrolizidinyl; indolyl-; dihydroindolyl-; isoindolyl-; indolizinyl-; indolizidinyl-; quinolyl-; dihydroquinolyl-; tetrahydroquinolyl-; isoquinolyl-; dihydroquinolyl-; tetrahydroquinolyl-; quinolizinyl-; quinolizidinyl-; phenanthrolinyl-; chromenyl-; chromanyl-; isochromenyl-; isochromanyl-; benzofuranyl-; and carbazolyl-groups; and the like.

14. The process of claim 1, wherein the aryl group is a substituted or an unsubstituted phenyl group.

15. The process of claim 1, wherein the cycloalkyl group is a cycloalkyl group with 5 to 7 carbon atoms

16. The process of claim 1, wherein the cycloalkenyl group is a cycloalkenyl group with 5 to 7 carbon atoms.

17. The process of claim 1, wherein the heterocyclic group has 5 or 6 carbon atoms.

18. The process of claim 1, wherein the enantiomeric mixture is a racemic mixture or a mixture of any ratio concentrations of the enantiomers.

19. The process of claim 1, which process includes adding to the reaction mixture water and at least one water-immiscible solvent.

20. The process of claim 1, which process includes adding to the reaction mixture water and at least one water-miscible organic solvent.

21. The process of claim 1, which process includes stopping the reaction when one enantiomer of the epoxide and/or vicinal diol is in excess compared to the other enantiomer of the epoxide and/or vicinal diol.

22. The process of claim 1, which process includes recovering continuously during the reaction the optically active epoxide and/or the optically active vicinal diol produced by the reaction directly from the reaction mixture.

23. The process of claim 1, wherein the yeast cell is of a yeast genus selected from the group consisting of Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, and Yarrowia.

24. The process of claim 1, wherein the yeast cell of a yeast species selected from the group consisting of Arxula adeninivorans, Arxula terrestris, Brettanomyces bruxellensis, Brettanomyces naardenensis, Brettanomyces anomalus, Brettanomyces species (e.g. NCYC 3151), Bullera dendrophila, Bulleromyces albus, Candida albicans, Candida fabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) rel to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Cryptococcus macerans, Debaryomyces hansenii, Dekkera anomala, Exophiala dermatitidis, Geotrichum species (e.g. UOFS Y-0111), Hormonema species (e.g. NCYC 3171), Issatchenkia occidentalis, Kluyveromyces marxianus, Lipomyces species (e.g. UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula species (e.g. UOFS Y-2042), Rhodotorula species (e.g. UOFS Y-0448), Rhodotorula species (e.g. NCYC 3193), Rhodotorula species (e.g. UOFS Y-0139), Rhodotorula species (e.g. UOFS Y-0560), Rhodotorula aurantiaca, Rhodotorula species (e.g. NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon species (e.g. UOFS Y-0861), Trichosporon species (e.g. UOFS Y-1615), Trichosporon species (e.g. UOFS Y-0451), Trichosporon species (e.g. NCYC 3212), Trichosporon species (e.g. UOFS Y-0449), Trichosporon species (e.g. NCYC 3211), Trichosporon species (e.g. UOFS Y-2113), Trichosporon species (e.g. NCYC 3210), Trichosporon moniliiforme, Trichosporon montevideense, Wingea robertsiae, and Yarrowia lipolytica.

25. A method for producing a polypeptide, which process includes the steps of:
providing a cell comprising a nucleic acid encoding and capable of expressing a polypeptide that has enantioselective styrene epoxide hydrolase activity;
culturing the cell; and
recovering the polypeptide from the culture.

26. The method of claim 25, wherein the cell is a yeast cell.

27. The method of claim 25, wherein the polypeptide is a full-length yeast epoxide hydrolase.

28. The method of claim 25, wherein the polypeptide is a functional fragment of a yeast epoxide hydrolase.

29. The method of claim 25, wherein the polypeptide is encoded by an endogenous gene of the cell.

30. The method of claim 25, wherein the cell is a recombinant cell and the polypeptide is encoded by a nucleic acid sequence with which the cell is transformed.

31. The method of claim 30, wherein the nucleic acid sequence is a heterologous nucleic acid sequence.

32. The method of claim 30, wherein the nucleic acid sequence is a homologous nucleic acid sequence.

33. A crude or pure enzyme preparation which includes an isolated polypeptide having enantioselective styrene epoxide hydrolase activity.

34. A substantially pure culture of cells, a substantial number of which comprise a nucleic acid encoding, and are capable of expressing, a polypeptide having enantioselective styrene epoxide hydrolase activity.

35. An isolated cell, the cell comprising a nucleic acid encoding a polypeptide having enantioselective styrene epoxide hydrolase activity, the cell being capable of expressing the polypeptide.

36. An isolated DNA comprising:
(a) a nucleic acid sequence that encodes a polypeptide that has enantioselective styrene epoxide hydrolase activity and that hybridizes under highly stringent conditions to the complement of a sequence selected from the group consisting of SEQ ID NOs: 6, 7, 8, 9, and 10; or
(b) the complement of the nucleic acid sequence.

37. The DNA of claim 36, wherein the nucleic acid sequence encodes a polypeptide comprising an amino acid sequence selected from the group consisting of SEQ ID NOs: 1, 2, 3, 4, and 5.

38. The DNA of claim 36, wherein the nucleic acid sequence is selected from the group consisting of SEQ ID NOs: 6, 7, 8, 9 and 10.

39. An isolated DNA comprising:
(a) a nucleic acid sequence that is at least 55% identical to a sequence selected from the group consisting of SEQ ID NOs: 6, 7, 8, 9 and 10; or
(b) the complement of the nucleic acid sequence,
wherein the nucleic acid sequence encodes a polypeptide that has enantioselective styrene epoxide hydrolase activity.

40. An isolated DNA comprising:
(a) a nucleic acid sequence that encodes a polypeptide consisting of an amino acid sequence that is at least 55% identical to a sequence selected from the group consisting of SEQ ID NOs: 1, 2, 3, 4 and 5; or
(b) the complement of the nucleic acid sequence,
wherein the polypeptide has enantioselective styrene epoxide hydrolase activity.

41. An isolated polypeptide encoded by the DNA of claim 32.

42. An isolated polypeptide comprising an amino acid sequence that is at least 55% identical to SEQ ID NOs: 1, 2, 3, 4, or 5, the polypeptide having enantioselective styrene epoxide hydrolase activity.

43. The polypeptide of claim 41, comprising:
a) an amino acid sequence selected from the group consisting of SEQ ID NOs; 1, 2, 3, 4, and 5, or a functional fragment of the sequence; or
b) the sequence of (a), but with no more than five conservative substitutions,
wherein the polypeptide has enantioselective styrene epoxide hydrolase activity.

44. An isolated antibody that binds to the polypeptide of claim 41.

45. The antibody of claim 44, wherein the antibody is a polyclonal antibody.

46. The antibody of claim 44, wherein the antibody is a monoclonal antibody.


SEQUENCE LISTING (28 sequences)

SEQ ID NO: 1 - protein, 409 aa, Rhodosporidium toruloides
MATHTFASPPTRFTVDIPQSELDELDFRLDKTRWPATEIVPEDGADDPTAFGLGAGPTLPLMKELAKGWREFDWKKAQDHLNTFEHYTVEIEDLSIHFLHHRSTRPNAVPLILCHGWPGHFGEFLNVIPLLTEPSDPSAQAFHVVAPSMPGYAWSSPPPSSKWSMPDTARVFDKLMTGLGYEKYMAQGGDWGSIAARCLGSLHKDHCKAVHLNFLPVFPPVPMWLINPHTLLAWAPRFLVPEKQAARMKRGLAYLEKGSAYYVMQQLTPRTPAYGLTDSPVGLLAWIGEKFEPTIQEASKQAQPTLTRDELYFTCSLYWFTRSIGTSFLPYSLNPHFTTFLTDSSTTCPTLPCPSTRARSTARQNGTLSVPATSSDQGRAGGRTLCCAREARRVCRPSQGGVRRHVGEV

SEQ ID NO: 2 - protein, 411 aa, Rhodosporidium toruloides
MKMATHTFASPPTRFTVDIPQSELDELHSRLDKTRWPATEIVPEDGTDDPTAFGLGAGPTLPLMKELAKGWREFDWKKAQDHLNTFEHYMVEIEDLSIHFLHHRSTRPNAVPLILCHGWPGHFGEFLNVIPLLTEPSDPSAQAFHVVAPSMPGYAWSLPPPSSKWNMPDTARVFDKLMTGLGYEKYMAQGGDWGSIAARCLGSLHKDHCKAVHLNFLPVFPPVPMWLINPHTLLAWAPRFLVPEKQAARMKRGLAYLEKGSAYYVMQQLTPRTPAYGLTDSPVGLLAWIGEKFEPTIQEASKQAQPTLTRDELYFTCSLYWFTRSIGTSFLPYSLNPHFTTFLTDSKYHLPNFALSLYPGEIYCPAERDAKRTGNLKWIKDAPEGGHFAALEKPDVFVEHLREAFGVMWEK

SEQ ID NO: 3 - protein, 410 aa, Rhodotorula araucariae
MSEHSFEAPPQPFTVDFAPHIEDLHRRLDNARWPTQEIVPVDVSETEHHNAFGLGMGPQLNLMKELANGWRAFDQSALQDHLNSFNNWKVEIEGLSIHFLHHRSTRAGALPLILCHGWPGGYHEFLHVVQLLTEPEGADAQAFHLVVPSMPGYAFSSPPPTAKWGMEDTARVFDKLMTGLGYNKYVAQGGDWGSITARCLGALHKDHCIAVHLNFCPVPPPFPFDQFNPRTLLNWMPRFVISDQQRAKLERGLAYLEKGSAYYVMQQLTPRTPAYALNDSPIGLLAWIGEKMIPGINEASAQPNPTLNRDALYTTLSLYWFTNSIGTSFLPYSLNPHFSTFLTSPRYRLPNFALSSFPGELFSPTPRDAARTGVMRWYKEADDGGHFAALEKPDVFSQHVREAVKAMLSS

SEQ ID NO: 4 - protein, 411 aa, Rhodosporidium paludigenum
MAAHSFTAPPAPYNIDFAPQVNDLHRRLDAARWPEHDVVPDDVDHGEHGAFGLGAGPSLALMKELAQEWRGQDQKQLQDHLNSYKNYRVEIEGLNIHFLHYPSSRADAFPLILCHGWPGGYHEFLHVLERLTEPEDQGSRAFHVVVPSMPGYAFSSPPKTAKWGMEDTARVFDKLMTGLGYAKYAAQGGDWGSITARCLGSLHKENCVAVHLNFCPVPPPFPLNMFNPRTLLDWMPRFVLPDQRRAKIERGVAYIERGSAYYAMQNLTPRTPAYGLNDSPIGLLAWIGEKMIPGIDKAVKHPNATLNREALFTTLSIYWFTGSIGSSFLPYALNPHFSTFLVSPRHQLPNFALSNFPDELFTPEERDARRTGNLRWYKDAEDGGHFAALEKPEVFAEHVREAMGVLLSNQA

SEQ ID NO: 5 - protein, 394 aa, Rhodotorula mucilaginosa
MPARSLTLRPFSPSFTAPELDGLARSLESSRLPAETYASRQAKYGIKHAWMKNALQRWKDGFDWKKHEQDINEVDHYMVQVQSDGIQHDLHVIYHESKDPNAIPLLLLHGWPGSAFEFIEAIKILRKSTSPAFHLIAPMEPGYGWSTPPPLDRGFNMNDCTALMNDLMVGLGYGDGYAAQGGDIGSGLARLLAVNYDACKCININYMPAVAPPEDAPERHQIKPHEEDALRRADEFQKTGRGYANMHATRPGTVGIVVGSSPVALLAWIAEKYLAWTDEDPPLDTILAICTIWWIRDSYPSSIWAYADFLETGISALHNDPKYKLDKKPFGFSSFKEEISATPEAWAGRNGNLQFYRYHDKGGHFAALEQPEAFAQDMQDCFGKIWPLSQEQKS

SEQ ID NO: 6 - DNA, 1230 bp, Rhodosporidium toruloides
atggcgacacacacattcgcttcgcctcccacgcgcttcaccgtcgacatcccacagtcagaactcgacgaactcgacttccgactcgacaagacccgctggccggcgacagagatcgttccagaggatggggcggacgacccgacggcgtttgggctcggagcagggccgacgctgccgctcatgaaggaactggcgaagggttggcgcgagttcgactggaagaaggcgcaggaccacctcaacacctttgagcactacacggtcgaaatcgaggacctctccatccacttcctccaccaccgctcgactcgcccgaatgctgttccgctcatcctctgccacggctggccaggccacttcggcgagttcctgaacgtcataccgctcttgacggagccgtcggacccgtccgcccaggcgttccacgtcgtcgcgccttcgatgcccggttatgcttggtcttcgcctcctccgtcctccaagtggagcatgcctgataccgcgagggtcttcgacaagctcatgaccgggcttggctacgagaagtacatggcgcagggcggagactggggcagcatcgctgctcgctgccttggatcgcttcacaaagaccactgcaaagccgtccacctcaacttcctccccgtcttcccacccgtcccgatgtggcttatcaacccgcacacgctccttgcctgggcaccgcgcttcctcgtgccggagaagcaggctgcgcgtatgaagcgcgggttggcgtaccttgagaagggctccgcctactacgtcatgcagcagttgacgcctcgcacgcctgcgtacggcctgaccgacagtcccgtcggcttgctggcctggatcggcgagaagttcgagccgaccattcaggaggcgagcaagcaagcccagccgaccctgactcgcgacgagctctacttcacctgctcgctctactggttcacccgctcaatcggcacctccttccttccctactcgctcaacccgcacttcaccaccttcctgaccgacagcaggtaccacctgcccaactttgccctgtccctctacccgggcgagatctactgcccggcagaacgggacgccaagcgtaccggcaacctcaagtggatcaaggacgcgcccgatggaggacactttgctgcgctcgagaagcccgatgtgtttgtcgagcatctcagggaggcgtttggcgtcatgtgggagaagtag

SEQ ID NO: 7 - DNA, 1230 bp, Rhodosporidium toruloides
atggcgacacacacattcgcttcgcctcccacccgcttcaccgtcgacatcccacagtcggaactcgacgaacttcactcgcgactcgacaagacccgctggccggcgacagagatcgttccagaggatgggacggacgatccgacggcgtttgggctcggagcagggccgacgctgccgctcatgaaggaattggcgaagggttggcgcgagttcgactggaaaaaggcgcaggaccacctcaacaccttcgagcactacatggtcgaaattgaggacctctcgatccacttcctccaccatcgctcgactcgcccgaacgctgttcccctcatcctctgccacggctggccaggccactttggcgagttcctgaacgttatcccgctcttgacggagccgtcggacccctccgctcaggcgttccacgtcgtcgccccttcgatgcctggctatgcttggtctttgcctcctccgtcctccaagtggaacatgcctgacaccgcgagggtcttcgacaagctcatgaccgggcttggctacgagaagtacatggcgcagggcggagactggggaagcatcgccgctcgctgccttggatcgctgcacaaggaccattgcaaagccgtccacctcaacttcctccccgtcttcccacccgtcccgatgtggcttatcaacccgcacacgctccttgcctgggcaccgcgcttcctcgtgccggagaagcaggctgcgcgtatgaagcgcgggttggcgtaccttgagaagggctccgcctactacgtcatgcagcagttgacgcctcgcacgcctgcgtacggcctgaccgacagtcccgtcggcttgctggcctggatcggcgagaagttcgagccgaccattcaggaggcgagcaagcaagcccagccgaccctgactcgcgacgagctctacttcacctgctcgctctactggttcacccgctcaatcggcacctccttccttccctactcgctcaacccgcacttcaccaccttcctgaccgacagcaagtaccacctgcccaactttgccctctcgctttacccaggcgagatctactgccccgccgagcgggacgccaagcgcaccggcaacctcaagtggatcaaggacgcgcctgagggaggacactttgctgcgctcgaaaagccggatgtgtttgtcgagcacctcagggaggcgtttggcgtcatgtgggagaagtag

SEQ ID NO: 8 - DNA, 1233 bp, Rhodotorula araucariae
atgagcgagcacagcttcgaggccccgccacagccgtttacggtcgactttgctccccacatcgaggatctccaccgccgtctcgacaatgcgcgctggccgacgcaagagattgtccccgtcgacgtgtccgagacggagcatcacaacgcgttcggactcgggatgggcccgcagctcaaccttatgaaggagctcgccaacggctggcgcgcgttcgaccagtcggcgctccaggaccacctcaacagcttcaacaactggaaggtcgagatcgagggattgtcgatccacttcctccaccatcgctcgacgcgcgccggcgctctcccgctcatcctgtgccatggctggcccggcgggtaccacgagttcctccacgtcgtccagctcctcaccgaaccagagggggcggatgcgcaggcgtttcacctcgtcgtcccctcgatgcccgggtacgccttctcgtctccgccgccgacggccaagtggggcatggaagacactgcaagggtttttgacaagctcatgaccggtttggggtacaacaagtatgtcgcgcagggcggtgactgggggtccatcacggcgcgatgcctcggcgcgctgcacaaggaccactgcattgctgtccacctcaacttctgccccgtcccgccgccgttcccattcgaccagttcaacccgcgcacgctgctcaactggatgccgcgcttcgtgatctcggaccagcagcgtgcgaagctcgagcgtgggctggcgtacctcgagaaggggtctgcttactatgtcatgcagcagctcacaccgcgtaccccggcctacgctctcaatgacagcccgattggcctgctcgcctggattggcgaaaagatgatcccaggcatcaacgaggcgagcgcgcagccgaacccgacgctcaatcgcgatgcgttgtacaccacgctctcgctgtactggttcaccaactccatcggcacctctttcctcccctactcgcttaacccgcacttcagcacgttcctcacctcgccccgctatcgcctgccgaactttgcgctgtcttccttcccgggcgagctgttctcgccgacgccgcgcgatgctgcgaggacgggcgtgatgcgctggtacaaggaggcggacgatggcgggcactttgcggcgctcgagaagcccgatgtgttcagccagcatgtcagggaggcagtcaaggccatgctgtcgtcgtga

SEQ ID NO: 9 - DNA, 1236 bp, Rhodosporidium paludigenum
atggctgcccattcctttactgcacctcctgcaccctacaacatcgactttgcgccccaggtaaatgacctgcaccgccgtctcgatgctgcccgctggccggaacacgacgtggtgcccgacgatgtggatcacggagagcacggcgcattcggactcggcgctggtcccagcctcgccctcatgaaggagctcgcgcaggaatggaggggccaggaccagaagcagctgcaggaccacctcaactcctacaagaactatcgcgtcgagatcgagggtctcaacatccacttcctgcactacccgtcgtctcgcgccgatgcgttcccgctcatcctgtgccacggctggcctggcggctaccacgagttcctgcacgtcctagagcgcctcacggagcccgaggatcaggggtcgcgggccttccatgtcgtcgtgccttccatgccgggttacgccttctcctcgccgcccaagacggcaaaatggggcatggaggacacggctcgcgtgttcgacaagctcatgacggggctaggttacgccaagtatgcggcccaaggcggtgactgggggtctatcacggcgcgctgcctaggttcgctgcacaaggagaactgcgtcgctgtccacctcaacttctgcccggttcccccgccgttcccgctcaacatgttcaacccgcgcacacttctggactggatgcctcgctttgtcctgcctgatcaacggcgggccaagattgagcgcggcgtggcctatatcgagcgcggctctgcctactacgccatgcaaaacttgacgccgcgcacgcctgcgtacggcttgaacgatagtccgattggtttgctcgcgtggattggcgagaagatgattccgggcattgacaaggctgtcaagcatccgaacgcaaccctcaatcgcgaagctcttttcacgacactctcgatctactggttcacgggctcgattggctcctccttcctgccatacgctctcaacccgcacttctctaccttcctcgtctcgccgcggcaccaactgccgaactttgctctgtccaactttcccgacgagctgttcacgcccgaagaacgcgatgctcgccgaaccggaaacttgcggtggtacaaggatgcagaggatggagggcacttcgcggcgctggagaagcccgaggtcttcgccgagcacgtaagggaggcgatgggggtcttgctgtcgaaccaggcctga

SEQ ID NO: 10 - DNA, 1185 bp, Rhodotorula mucilaginosa
atgcccgcccgctcgctcacgctgcgcccgttctcgccgtcgttcacggctccggaactggacggtctcgctcgctcgctcgagtcgtcgcgcttgcccgccgagacgtacgcttcccgccaggccaaatacggcatcaagcatgcttggatgaagaatgccctccaacggtggaaggacgggttcgattggaagaagcacgagcaggacatcaacgaggtcgaccactatatggtgcaggtccagtccgatggcattcaacacgacctccatgtgatctatcacgaatcgaaagacccgaatgcgatcccgctcttgctgctgcatgggtggcccggttccgcgttcgagtttatcgaggcgatcaagatccttcgcaagagtacctcgcccgcgttccacctgatcgcgcccatggagcccggctacgggtggagtactccgccgccactcgaccgcggtttcaacatgaacgattgcacagcgctcatgaacgacttgatggttggactcgggtacggagacggttacgctgctcagggtggcgacatcggttcgggactcgcaagactcctcgccgtcaactatgacgcatgcaaatgcatcaacatcaactacatgcctgccgttgcaccaccagaggacgctccggagcggcaccagatcaaaccgcacgaggaggatgcgctccgacgtgcggacgagtttcagaagacgggcagggggtatgccaacatgcatgcaacgagacccggtacggtcggcatcgtcgtcggtagttcgccggtcgcactgctcgcttggatcgctgaaaagtacctcgcttggaccgatgaggatccgcccctcgacacgatcctcgcaatctgcaccatctggtggatccgcgactcgtacccttcttcaatctgggcctacgccgactttctcgagacgggcatctcggccctgcacaacgacccgaagtacaaacttgacaagaaaccgttcgggttctcgagcttcaaggaggagatcagcgcgactcccgaggcgtgggcgggcaggaacggcaacttgcagttctatcggtaccacgacaagggaggtcactttgcggcgctcgagcagccggaagcgttcgcgcaagacatgcaggattgcttcggcaaaatctggcctctctctcaggagcaaaaatcgtag

SEQ ID NO: 11 - DNA, 16 bp, Artificial Sequence
  Primer
ggagttcttcgcccac

SEQ ID NO: 12 - DNA, 17 bp, Artificial Sequence
  Primer
gatccccaccggaattg

SEQ ID NO: 13 - DNA, 20 bp, Artificial Sequence
  Primer
catacaaccacacacatcca

SEQ ID NO: 14 - DNA, 21 bp, Artificial Sequence
  Primer
taaatagcttagataccacag

SEQ ID NO: 15 - DNA, 19 bp, Artificial Sequence
  Primer
ctctctctccttgtcaact

SEQ ID NO: 16 - DNA, 21 bp, Artificial Sequence
  Primer
gtggatccatggcgacacaca

SEQ ID NO: 17 - DNA, 22 bp, Artificial Sequence
  Primer
acaccctcttcatcggatccag

SEQ ID NO: 18 - DNA, 23 bp, Artificial Sequence
  Primer
gattaatgatcaatgagcgagca

SEQ ID NO: 19 - DNA, 20 bp, Artificial Sequence
  Primer
gacagcagcactggatccag

SEQ ID NO: 20 - DNA, 19 bp, Artificial Sequence
  Primer
gtggatccatggctgccca

SEQ ID NO: 21 - DNA, 18 bp, Artificial Sequence
  Primer
ggtccggactcgatcgag

SEQ ID NO: 22 - DNA, 21 bp, Artificial Sequence
  Primer
gtatatctatgcccgcccgct

SEQ ID NO: 23 - DNA, 22 bp, Artificial Sequence
  Primer
tcgtttttagcatcggatccag

SEQ ID NO: 24 - DNA, 21 bp, Artificial Sequence
  Primer
gcagatctatgtcatcactcg

SEQ ID NO: 25 - DNA, 22 bp, Artificial Sequence
  Primer
gcagcttcaacatcggatccag

SEQ ID NO: 26 - protein, 409 aa, Cryptococcus curvatus
MATHTFASPPTRFTVDIPQSEVDQLHSRLDKTRWPGTEIVPEDGADDPTAFGLGAGPTLPLMKELAKGWRDFDWKKAQDHLNTFEHYTVEIEDLSIHFLHHRSTRPNAVPLILCHGWPGHFGEFLHVIPLLTEPSDPSAQAFHVVAPSMPGYAWSSPPPTSKWNMPDTARVSDKLMNGLGYEKYMAQGGDWGSIAARCLGALHKDHCKAVHLNFLPVFPPVPMWLINPHTLLAWAPRFLVPEKQAARMKRGLAYLEKGSAYYVMQQLTPRTSAYGLTDSPVGLLAWIGEKFEPTIQEASKQAQPTLTRDELYFTCSLYWFTRSIGTSFLPYSLNPHFTTFLTDSKYYLPNFALSLYPGEIYCPAERDAKRTGNLKWIKDAPEGGHFAALEKPDVFVDHLREAFGVMWEK

SEQ ID NO: 27 - protein, 354 aa, Yarrowia lipolytica
MTDSLSLGYYQLFHKYAVLGDKRWHYLDIPPTSPSPALLGSGKTLMLCHGFPDSWYGWRKQIPILRNLGYRLLVPSQMGYTRSEAPLYPTPEPNEKGEYPELGLEDGTDALKDLYCYTGKFYAQCMDELLTQLGIPTVTIIGHDYGAYLGPKLYLYFPHRVEAIATSCWHYVPALKKFFAIPDFIKLAPSLSYQAYLIGDASKDFATREGSEPFLRQVFGGEGWGGSDSSVHMTEPEFQNYMTEFATGGRFLSYMVSTYKARRLTFEIEKQDFLDKGRTPESMQVDVPYLHVGAEQDMAFRPPMIKNLRKYVKPGRLDEVWIDASHWLMFEKADEFNHELVKWLDKIHGSQSKL

SEQ ID NO: 28 - protein, 401 aa, Filobasidium neoformans var neoformans
MSYSDLPHKPTIPVEPFKLSVPHEDLNGLLTLLKSTRIAKESYENVSAHENKFGITRKWLVNMKDEWIKQDWRKQEERINSLPAFKAKVKNSDGSVFSIHFTALFSKKKVAIPIILSHGWPGSFYEFVPMMEMVKKKYSPEDLPFHLIVPSLPGWLFSTPPPNDREFNVTDVGYLFNGLMEGLGFGDGYVAQGGDIGSYVTNELGAKYPACKIIHVNYSNPPPRPLPSPGSPGQEASPPSAEDLLELLQKFGYALEHSTRPATVGLVVGSNPLSLLAWVGEKFLEWTDESPSEETILTMTSLYWFTDCFTTSIYTYRYGLGAKRHESAKQASYQKCPLGYSQFPKEIVEIPAEWVKAQSNMVWSKKHESGGHFAALEKPELLWADIEEFVHSQWENYKGNY